(19) United States
(12) Patent Application Publication
(10) Pub. No.: US 2025/0109403 A1
(43) Pub. Date: Apr. 3, 2025

(54) SYSTEMS AND METHODS FOR MODIFYING GRAPE BERRY AND PLANTLET COLOR

(71) Applicant: Sun World International, LLC, Bakersfield, CA (US)

(72) Inventor: Chenxing Niu, Bakersfield, CA (US)

(21) Appl. No.: 18/900,351

(22) Filed: Sep. 27, 2024

Related U.S. Application Data
(60) Provisional application No. 63/587,061, filed on Sep. 29, 2023.

(51) Int. Cl. C12N 15/82; C12N 15/11

(52) U.S. Cl. CPC C12N 15/825; C12N 15/11; C12N 15/8213; C12N 2310/20

(57) ABSTRACT

The present technology provides targeted genome editing techniques to modify the color of Vitis plant parts and fruits thereof. In particular, the present technology relates to the use of genome editing methods to generate edits resulting large inactivating deletions in genes of interest, such as genes and genetic elements associated with plant color and pigmentation (e.g., PDS1 gene, mybA1 gene, and Gret1 retrotransposon), to modify plant and fruit color traits.

### CROSS-REFERENCE TO RELATED APPLICATIONS

This application claims the benefit of and priority to U.S. Provisional Patent Application No. 63/587,061, filed Sep. 29, 2023, the entire contents of which is incorporated herein by reference in its entirety for any and all purposes.

### SEQUENCE LISTING

The instant application contains a Sequence Listing which has been submitted electronically in XML file format and is hereby incorporated by reference in its entirety. Said XML copy, created on Sep. 26, 2024, is named 132554-0129_SL.xml and is 100,785 bytes in size.

### TECHNICAL FIELD

The present technology relates generally to the use of targeted gene editing techniques to modify the color of grape (Vitis spp., such as Vitis vinifera L.) plantlet and berries. In particular, the present technology relates to the use of gene editing methods to generate edits of genes or genetic elements of interest, such as the mybA1 gene and the Gret1 retrotransposon, to edit genes or genetic elements controlling plantlet and berry color in plants for producing plants and berries having a desired color.

### BACKGROUND

The following description is provided to assist the understanding of the reader. None of the information provided or references cited is admitted to be prior art.

Grapevine (Vitis vinifera L.) is one of the most economically important fruit crops and is used as table fruit, dried raisins, and for juice or wine. Grape berry color is an economically important trait that is determined by the differential accumulation of anthocyanins in the epidermal and sub-epidermal cell layers of the berry skin and flesh. Anthocyanins in grape berries are synthesized in an extension of the general flavonoid biosynthetic pathway.

Both genetic and environmental factors play a role in the final berry color. Several genes, many of which encode enzymes of the anthocyanin biosynthetic pathway, play a role in grape berry color determination. The enzymes, phenylalanine ammonia lyase (PAL), chalcone synthase (CHS), chalcone isomerase (CHI), flavanone-3β-hydroxylase (F3H), flavonoid-3′-hydroxylase (F3′H), flavonoid-3′5 hydroxylase (F35′H), and dihydroflavonal-4-reductase (DFR) are each responsible for the synthesis of a different precursor in the anthocyanin biosynthetic pathway. Leucoanthocyanidin dioxygenase (LDOX) is responsible for the production of anthocyanins cyanidin and delphinidin from leucocyanidin and leucodelphinidin through oxidation. S-adenosyl-L-methionine (SAM) or O-methyltransferase (OMT) methylate the anthocyanins to create peonidin, petunidin, and malvidin. Anthocyanidin synthase (ANS) also plays a pivotal role in the biosynthesis of both anthocyanins and proanthocyanidins Two genes indirectly involved in the pathway are VvMYBAI and VvMYBA2, which are transcription factors that regulate the anthocyanin biosynthetic pathway by modulating expression of the UDP-glucose:flavonoid 3-O-glucosyltransferase (UFGT) gene. Mutations in MYBA1 and MYBA2 genes can cause a loss of transcription factor activity on anthocyanin biosynthetic genes, leading to a “white” phenotype. A genetic element that may exert control of fruit color is the retrotransposon Gret1. The insertion of a ˜10-kb Gret1 transposon in the promoter of mybA1 has been shown to block the gene expression of mybA1 and reduce the amount of anthocyanin pigments, resulting in a non-functional mybA1 allele.

Grape berry color is an important trait that impacts the end use of the fruit, and, as such, has significant implications for both consumers and grape growers. Berry color is a critical breeding target for both wine and table grapes. Certain consumers may prefer deeply colored fruits, such as red grapes, for the nutritional properties that they possess. For grape growers, producing grapes of a desired berry color is critical for the marketability of their products.

Accordingly, there is a need to develop improved, precise genome targeting technologies for altering the expression of gene targets influencing grape berry color that are affordable, scalable, amenable to targeting multiple positions within the genome, and that can be used for the modulation of grape berry color in grapevine, cell lines, and derivatives thereof, to assist grape growers and breeders with targeting desirable color profiles and breeding for them more efficiently.

### SUMMARY

Disclosed herein are methods and compositions for plantlet and berry color in plants. In particular, disclosed herein are targeted genome editing methods and compositions for altering the expression of one or more genes involved in determining plantlet and/or berry color in plants (e.g., grape plants, e.g., Vitis plants).

In one aspect, the present disclosure provides a method for producing an edited genome in a plant cell of genus Vitis, the method comprising introducing into the cell a Clustered Regularly Interspaced Short Palindromic Repeats (CRISPR)-associated (CRISPR-Cas) system comprising: (a) at least two guide RNAs (gRNA), or at least one polynucleotide encoding the at least two gRNAs, and (b) an effector protein, or one or more polynucleotides encoding the effector protein; wherein each of the at least two gRNAs hybridize to a Gret1 sequence, and each of the at least two gRNAs form a complex with the effector protein; and wherein the effector protein is a Cas protein comprising a nuclease and/or an effector domain. In some embodiments, the Cas protein is a Cas9 protein, a Cpf1 protein, or a Csm1 protein. In some embodiments, the edited genome results in a change in the color of a plantlet produced by a plant derived from the plant cell from green to red. In some embodiments, the edited genome results in a change in the color of a fruit produced by a plant derived from the plant cell from green to red. In some embodiments, a plantlet produced by a plant derived from the plant cell comprising the edited genome is red, and wherein a plantlet produced by a plant derived from a plant cell that does not comprise the edited genome is green. In some embodiments, a fruit produced by a plant derived from the plant cell comprising the edited genome is red, and wherein a fruit produced by a plant derived from a plant cell that does not comprise the edited genome is green.

In one aspect, the present disclosure provides a method for producing an edited genome in a plant cell of genus Vitis, the method comprising introducing into the cell a Clustered Regularly Interspaced Short Palindromic Repeats (CRISPR)-associated (CRISPR-Cas) system comprising: at least one guide RNA (gRNA), or at least one polynucleotide encoding the at least one gRNA, and an effector protein, or one or more polynucleotides encoding the effector protein; wherein the at least one gRNA hybridizes to a Gret1 sequence, and the at least one gRNA forms a complex with the effector protein; and wherein the effector protein is a Cas protein comprising a nuclease and/or an effector domain. In some embodiments, the Cas protein is a Cas9 protein, a Cas12a protein, a Cpf1 protein, or a Csm1 protein. In some embodiments, the Cas protein is a Cas12a protein. In some embodiments, the edited genome results in a change in the color of a plantlet produced by a plant derived from the plant cell from green to red. In some embodiments, the edited genome results in a change in the color of a fruit produced by a plant derived from the plant cell from green to red. In some embodiments, a plantlet produced by a plant derived from the plant cell comprising the edited genome is red, and wherein a plantlet produced by a plant derived from a plant cell that does not comprise the edited genome is green. In some embodiments, a fruit produced by a plant derived from the plant cell comprising the edited genome is red, and wherein a fruit produced by a plant derived from a plant cell that does not comprise the edited genome is green. In some embodiments, the at least one gRNA comprises a nucleotide sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35. In some embodiments, the at least one gRNA comprises SEQ ID NO: 26. In some embodiments, the at least one gRNA comprises SEQ ID NO: 28. In some embodiments, the at least one gRNA comprises SEQ ID NO: 32. In some embodiments, the system comprises at least two gRNAs. In some embodiments, the at least two gRNAs comprise SEQ ID NO: 26 and SEQ ID NO: 33. In some embodiments, the at least two gRNAs comprise SEQ ID NO: 34 and SEQ ID NO: 35.

In some embodiments, the plant cell is a SUGRA35 cell. In some embodiments, the Cas protein is optimized for expression in the Vitis cell.

In one aspect, the present disclosure provides a genetically engineered Vitis cell produced by any of the above methods.

In one aspect, the present disclosure provides a genetically engineered Vitis plant comprising the cells produced by any of the above methods.

In one aspect, the present disclosure provides a product comprising any of the above-described genetically engineered plants, or portions thereof, wherein the product has altered pigmentation relative to a product produced by a non-genetically engineered plant of the same strain. In some embodiments, the product comprises a fruit. In some embodiments, the product comprises a grape. In some embodiments, the product is a wine.

In one aspect, the present disclosure provides a guide RNA (gRNA) comprising the nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

In one aspect, the present disclosure provides a composition comprising: (a) a polynucleotide comprising a sequence encoding a guide RNA (gRNA) having a nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35; and (b) a polynucleotide comprising a sequence encoding a Cas enzyme.

The technologies described and claimed herein have many attributes and embodiments including, but not limited to, those set forth or described or referenced in this brief summary. It is not intended to be all-inclusive and the technology described and claimed herein are not limited to or by the features or embodiments identified in this brief summary, which is included for purposes of illustration only and not restriction. Additional embodiments may be disclosed in the brief description of the drawings and detailed description below.

### BRIEF DESCRIPTION OF THE DRAWINGS

FIG. 1A is an illustration that shows a pHEE-AT5G plasmid vector. The region at the top of the plasmid, between the dotted lines, is a gRNA1- and gRNA2-containing region for targeting a PDS1 or mybA1 gene. This region is also referred to as a gRNA1&2 related piece, and is cloned out of the pHEE-AT5G plasmid vector using forward and reverse primers to create new gRNAs for targeting PDS1 or mybA1 genes.

FIG. 1B is an illustration showing a BsaI-digested pHEE vector backbone into which a gRNA1&2 piece containing gRNAs directed to a PDS1 gene or a mybA1 gene were cloned.

FIG. 2A is plasmid map of a pHEE401E vector.

FIG. 2B is a plasmid map of a pHDE vector backbone.

FIG. 3A shows a sequencing confirmation analysis, confirming insertion of a PDS1-targeting gRNA1&2 piece into a pHEE backbone. Figure discloses SEQ ID NOS 22-25, respectively, in order of appearance.

FIG. 3B shows a gel confirmation of a large deletion of PDS1 gene in the callus stage using the CRISPR-cas9-based gene editing vector. When no edit occurs, the PCR using PDS1-GT1 and PDS1-GT3 will show a band around 600 bp, and the PCR using PDS1-GT1 and PDS1-GT2 will not show a band; when editing occurs, the PCR using PDS1-GT1 and PDS1-GT3 will not show a band, and the PCR using PDS1-GT1 and PDS1-GT2 will show a band around 800 bp. The left side of the figure shows the PCR products of PDS1-GT1 and PDS1-GT3: callus 7 and 8 appear to have been edited. The right side of the figure shows the PCR products of PDS1-GT1 and PDS1-GT2, callus 7 and 8 appear to have been edited.

FIG. 3C is a representative image of the wild-type phenotype of Vitis vinifera ‘Thompson Seedless’ plantlet, which is green in color.

FIG. 3D is a representative image of the albino phenotype of a PDS1-edited Vitis vinifera ‘Thompson Seedless’ plantlet.

FIG. 3E is a representative image of the wild-type phenotype of Vitis vinifera ‘Thompson Seedless’ plantlet leaf, which is green in color.

FIG. 3F is a representative image of the mosaic phenotype of a Vitis vinifera ‘Thompson Seedless’ PDS1-edited plantlet leaf.

FIG. 3G is an image showing a comparison between pHEE-PDS1 edited plantlets and the non-edited plantlets in the center petri dish.

FIG. 3H is an image showing a comparison between pHDE-PDS1 edited plantlets (albino phenotype, predominantly in the petri dishes on the left side of the image) and the non-edited plantlets (green phenotype, predominantly in the petri dishes on the right side of the image).

FIG. 4A is an image showing representative phenotypes of wildtype anthocyanin over-expressed Thompson Seedless (AOTS) plantlet (red; leftmost plantlet shown in plate) compared to MybA1-edited AOTS plantlets (green).

FIG. 4B is an image showing MybA1 edited AOTS plantlets vs. non-edited plantlets in petri dishes.

FIG. 4C is an image showing wildtype Merlot fruits at early stage (mixed green and purple colored fruits).

FIG. 4D is an image showing wildtype Merlot fruits at mature stage (mostly dark purple colored fruits).

FIG. 4E is an image showing fruits from a MybA1-edited Merlot plant at early stage (green colored fruits).

FIG. 4F is an image showing fruits from a MybA1-edited Merlot plant at mature stage (green colored fruits).

FIG. 5 is a schematic illustrating the relative positions of sequences of Gret1 that can be targeted by gRNAs of the present disclosure.

### DETAILED DESCRIPTION

### I. Introduction

The present technology encompasses the use of targeted genome engineering (also known as genome editing) techniques that can be used to generate a gene edit, such as a gene edit resulting in a deletion of a genes of interest in order to eliminate the expression of the protein products of the genes, in plants. For example, in some embodiments, the present technology contemplates the introduction of a large deletion of a gene of interest, resulting in a non-functional gene product. In some embodiments, the gene edits are generated using the genome editing methods provided herein. Programmable nucleases enable precise genome editing by introducing DNA double strand breaks (DSBs) at specific genomic loci. DSBs subsequently recruit endogenous repair machinery for either non-homologous end-joining (NHEJ) or homology directed repair (HDR) to the DSB site to mediate genome editing. When the DSBs are repaired by either NHEJ or HDR, the sequence at the repair site can be modified or new genetic information can be inserted (e.g., donor DNA comprising a desired gene edit can be inserted into the target gene at the break site). Methods involving the use of programmable nucleases include the CRISPR (clustered regularly interspaced short palindromic repeats)/Cas (CRISPR-associated) system, meganucleases and their derivatives, zinc finger nucleases (ZFNs), and transcription activator like effector nucleases (TALENs). ZFNs, TALENs, and meganucleases achieve specific DNA binding via protein-DNA interactions. Cas9 is targeted to specific DNA sequences by a short RNA guide molecule that base-pairs directly with the target DNA.

Vitis cells and plants modified by the methods described herein are characterized by altered color or pigment profile when compared to unmodified counterpart plants. In some embodiments, one or more portions of a Vitis plant modified by the methods described herein are characterized by altered color or pigment profile when compared to unmodified counterpart plants. For example, in some embodiments, modified plants comprise altered plantlet color. In some embodiments, modified plants comprise altered fruit (e.g., grape) color.

Vitis plants according to the present technology with reduced expression of one or more of the genes involved in plantlet and/or fruit color determinations will be desirable in the production of Vitis products having altered color content. Vitis plants according to the present technology will be suitable for use in any Vitis product, including but not limited to whole grapes, freeze-dried fruit (e.g., grape), raisin, wine, fruit juice (e.g., grape juice or another juice comprising a product derived from fruits of plants described herein), nutritional products, sustenance products, puree, pastes, and fruit leathers.

A number of genes and genetic elements are known to contribute to the pigmentation of various parts of the Vitis plant, such as leaves, callus, and fruit. An example of a gene associated with Vitis pigmentation is MybA1. The MybA1 gene in Vitis encodes a transcription factor, belonging to the R2R3 Myb family, that regulates the anthocyanins biosynthesis pathway, and thereby regulates anthocyanin pigment production. Mutations in MybA1 can cause a loss of transcription factor activity on anthocyanin biosynthetic genes, leading to a ‘white’ phenotype.

An example of a genetic element associated with Vitis pigmentation is grape retrotransposon 1 (Gret1). Gret1 is a transposon found in the promoter of the Vitis vinifera MybA1 gene. It has been hypothesized that the loss of berry skin pigmentation has been due to the insertion of the Gret1 retrotransposon in the 5′ regulatory region of the MybA1 gene (Kobayashi S. et al. Retrotransposon-induced mutations in grape skin color. Science. 2004; 304:982).

### II. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant.

“Genetic element” as used herein, refers to genetic material that can move around within a genome. Non-limiting examples of genetic elements include transposons, DNA transposons, retrotransposons, integrons, introns, and introners.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores. The class of plants that can be used in the present technology is generally as broad as the class of higher plants amenable to transformation techniques, including both monocotyledonous and dicotyledonous plants. In some embodiments, the plant is a fruit-bearing plant. In some embodiments, the plant is a plant of the genus Vitis.

“Loss of function” refers to the loss of function of one or more of the color-associated genes described herein in a host tissue or organism, and encompasses the function at the molecular level and also at the phenotypic level (e.g., altered color in a plant or plant part).

The terms “modification,” “genomic modification,” “modified nucleotide,” or “edited nucleotide” as used herein refer to a nucleotide sequence of interest that comprises at least one alteration when compared to its non-modified nucleotide sequence. Such “alterations” include, for example: (i) replacement or substitution of at least one nucleotide, (ii) a deletion of at least one nucleotide, (iii) an insertion of at least one nucleotide, or (iv) any combination of (i)-(iii). In some embodiments, such modifications to a gene reduce or eliminate the expression of the gene product and/or its activity.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” or “operatively linked” refers to a functional linkage between a promoter and a second sequence, where the promoter sequence initiates and mediates transcription of the DNA sequence corresponding to the second sequence. In general, “operably linked” or “operatively linked” means that the nucleic acid sequences being linked are contiguous. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame). Non-limiting examples of promoters useful in the present technology include an Arabidopsis thaliana U6 RNA polymerase III promoter, a 35S promoter, ubiquitin promoter, an EC1/EC2 promoter, Rubisco small subunit promoter, an inducible promoter, including, but not limited to, an AlcR/AlcA (ethanol inducible) promoter, a glucocorticoid receptor fusion, GVG, a pOp/LhGR (dexamethasone inducible) promoter, an XCE/OlexA promoter, a heat shock promoter, and or a bidirectional promoter.

A “region of interest” is any region of cellular chromatin, such as, for example, a gene or a non-coding sequence within or adjacent to a gene, in which it is desirable to bind an exogenous molecule. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

As used herein, “homology” or “identity” or “similarity” refers to sequence similarity between two nucleic acid molecules or two peptides. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. Two sequences are deemed “unrelated” or “non-homologous” if they share less than 40% identity, or less than 25% identity, with each other.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, California, USA). A polynucleotide or polynucleotide region (or a polypeptide or polypeptide region) has a certain percentage (for example, at least 60%, 65%, 70%, 75%, 80%, 85%, 90%, 95%, 96%, 97%, 98%, 99%, or 100%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST. Details of these programs can be found at the National Center for Biotechnology Information. Biologically equivalent polynucleotides are those having the specified percent homology and encoding a polypeptide having the same or similar biological activity.

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant.

“Grape plant” refers to any species in the Vitis genus that produces grapes, including but not limited to the following: Vitis acerifolia, Vitis aestivalis, Vitis amurensis, Vitis arizonica, Vitis baihuashanensis, Vitis balansana, Vitis bashanica, Vitis bellula, Vitis berlandieri, Vitis betulifolia, Vitis biformis, Vitis blancoi. Vitis bloodworthiana, Vitis bourgaeana, Vitis bryoniifolia, Vitis californica, Vitis×champinii, Vitis chunganensis, Vitis chungii, Vitis cinerea, Vitis coignetiae, Vitis davidi, Vitis×doaniana, Vitis erythrophylla, Vitis fengqinensis, Vitis ficifolia, Vitis flavicosta, Vitis flexuosa, Vitis girdiana, Vitis hancockii, Vitis heyneana, Vitis hissarica, Vitis hui, Vitis jaegeriana, Vitis jinggangensis, Vitis jinzhainensis, Vitis kiusiana, Vitis lanceolatifoliosa, Vitis longquanensis, Vitis luochengensis, Vitis menghaiensis, Vitis mengziensis, Vitis metziana, Vitis monticola, Vitis mustangensis, Vitis nesbittiana, Vitis×novae-angliae, Vitis novogranatensis, Vitis nuristanica, Vitis palmate, Vitis pedicellata, Vitis peninsularis, Vitis piasezkii, Vitis pilosonervia, Vitis popenoei, Vitis pseudoreticulata, Vitis qinlingensis, Vitis retordii, Vitis romanetii, Vitis ruyuanensis, Vitis saccharifera, Vitis shenxiensis, Vitis shuttleworthii, Vitis silvestrii, Vitis sinocinerea, Vitis sinoternata, Vitis tiliifolia, Vitis tsoi, Vitis vinifera, Vitis wenchowensis, Vitis wenxianensis, Vitis wilsoniae, Vitis wuhanensis, Vitis xunyangensis, Vitis yunnanensis, Vitis zhejiang-adstricta, and interspecific hybrids of the above. While the methods and products of the present disclosure may be used on any grape variety, Table 1 lists a number of commercial varieties as specific examples of varieties that may be modified according to the methods of the present disclosure to make genetically engineered plants and products of the present disclosure. For example: Sugrathirtyfive is a green variety that may be edited according to the methods of the present disclosure to become red or black; Sugrathirteen is a black variety that may be edited according to the methods of the present disclosure to become green or red; and Sugrafiftythree is a red variety that may be edited according to the methods of the present disclosure to become green or black.

TABLE 1
Exemplary grape plant varieties

| Variety | | |
|---|---|---|
| (Vitis vinifera) | Nickname | Fruit Color |
| Sugraone | SUGRA1 | Green |
| Sugrafive | SUGRA5 | Green |
| Sugratwelve | SUGRA12 | Green |
| Sugrathirteen | SUGRA13 | Black |
| Sugrafourteen | SUGRA14 | Red |
| Sugrafifteen | SUGRA15 | Green |
| Sugrasixteen | SUGRA16 | Black |
| Sugraseventeen | SUGRA17 | Red |
| Sugraeighteen | SUGRA18 | Green |
| Sugranineteen | SUGRA19 | Red |
| Sugratwenty | SUGRA20 | Red |
| Sugratwentyone | SUGRA21 | Red |
| Sugratwentytwo | SUGRA22 | Green |
| Sugratwentythree | SUGRA23 | Black |
| Sugratwentyfour | SUGRA24 | Green |
| Sugratwentysix | SUGRA26 | Green |
| Sugratwentyseven | SUGRA27 | Black |
| Sugratwentyeight | SUGRA28 | Black |
| Sugrathirtyone | SUGRA31 | Green |
| Sugrathirtytwo | SUGRA32 | Red |
| Sugrathirtythree | SUGRA33 | Green |
| Sugrathirtyfour | SUGRA34 | Black |
| Sugrathirtyfive | SUGRA35 | Green |
| Sugrathirtysix | SUGRA36 | Red |
| Sugrathirtyeight | SUGRA38 | Red |
| Sugrathirtynine | SUGRA39 | Green |
| Sugraforty | SUGRA40 | Black |
| Sugrafortyone | SUGRA41 | Green |
| Sugrafortytwo | SUGRA42 | Green |
| Sugrafortythree | SUGRA43 | Green |
| Sugrafortyfive | SUGRA45 | Black |
| Sugrafortysix | SUGRA46 | Red |
| Sugrafortyseven | SUGRA47 | Red |
| Sugrafortyeight | SUGRA48 | Black |
| Sugrafortynine | SUGRA49 | Black |
| Sugrafifty | SUGRA50 | Red |
| Sugrafiftyone | SUGRA51 | Green |
| Sugrafiftytwo | SUGRA52 | Green |
| Sugrafiftythree | SUGRA53 | Red |
| Sugrafiftyfour | SUGRA54 | Green |
| Sugrafiftyfive | SUGRA55 | Red |
| Sugrafiftysix | SUGRA56 | Green |
| Sugrafiftyseven | SUGRA57 | Red |
| Sugrafiftyeight | SUGRA58 | Red |
| Sugrafiftynine | SUGRA59 | Red |
| Sugra60 | SUGRA60 | Red |
| Sugra61 | SUGRA61 | Red |
| Sugra62 | SUGRA62 | Green |
| Sugra63 | SUGRA63 | Green |
| Sugra64 | SUGRA64 | Red |
| Sugra65 | SUGRA65 | Red |
| Sugra66 | SUGRA66 | Red |
| Sugra67 | SUGRA67 | Red |
| Sugra68 | SUGRA68 | Red |
| Sugra69 | SUGRA69 | Green |
| Sugra70 | SUGRA70 | Red |

As used herein, “transformation” refers to the introduction of exogenous nucleic acid into cells, so as to produce transgenic cells stably transformed with the exogenous nucleic acid.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

As used herein, the terms “vector,” “vehicle,” “construct,” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

### III. Targeted Genome Engineering Of Plants And Cells To Reduce Expression Of Endogenous Genes Controlling Plantlet and/or Berry Color

The present technology contemplates methods and compositions for altering plantlet and/or berry color in plants. In particular, the present technology relates to targeted genome engineering (also known as genome editing) methods and compositions for altering the expression of one or more genes encoding proteins involved in plantlet and/or berry color determination. Provided herein are methods and compositions for modifying a target genomic locus in a cell to modulate the expression of one or more gene products involved in plantlet and/or berry color determination. Targeted genome engineering techniques described herein include the CRISPR (clustered regularly interspaced short palindromic repeats)/Cas (CRISPR-associated) system, meganucleases, zinc finger nucleases (ZFNs), and TAL effector nucleases (TALENs). Such techniques may be employed to bind to and/or cleave a genomic region of interest of or adjacent to one or more genes involved in plantlet or berry color determination. In some embodiments, the genome editing techniques described herein generate a specific sequence change or gene edit (e.g., insertion, deletion, or substitution) in the 5′-UTR of a gene involved in plantlet and/or berry color determination, such as generating a single nucleotide gene edit to form an out-of-frame start codon upstream of the gene's ORF, thereby suppressing expression of the gene involved in plantlet and/or berry color determination. In some embodiments, the gene edit (e.g., deletion, insertion, or substitution) results in production of an upstream, out-of-frame start codon that may result in the elimination of protein production or a nonfunctional protein. In some embodiments, the genome editing techniques described herein generate a specific sequence change or gene edit (e.g., insertion, deletion, or substitution) in the coding region or a non-coding region of a gene involved in plantlet and/or berry color determination, such as generating a large deletion to form (1) an out-of-frame start codon upstream of the gene's ORF, thereby suppressing expression of the gene involved in plantlet and/or berry color determination, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases. In some embodiments, the large deletion is generated in a PDS1 gene. In some embodiments, the large deletion is generated in a mybA1 gene. In some embodiments, the large deletion is generated in a Gret1 sequence. In some embodiments, a large deletion is a complete deletion of a gene or genetic element. For example, in some embodiments, a Gret1 sequence is deleted.

### Genome Editing Systems

### CRISPR/Cas Systems

In some embodiments, the methods of the present technology relate to the use of a CRISPR/Cas system that binds to a target site in a region of interest in a genome, wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering in plant cells.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Cys3, Cse1, Cse2, Csc1, Csc2, Csa5, Csn2, Csm1, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcus pyogenes Cas9 protein. In some embodiments, the Cas protein is a Cas12a (Cpf1) protein. In some embodiments, the Cas protein is a Csm1 protein. These enzymes are known. For example, the amino acid sequence of S. pyogenes Cas9 protein may be found in the SwissProt database under accession number Q99ZW2. The amino acid sequence of Francisella tularensis subsp. Novicida Cpf1 protein may be found in the UniProt database under accession number A0Q7Q2. The amino acid sequence of Thermococcus onnurineus Csm1 protein may be found in the UniProt database under accession number B6YWB8.

The sgRNA molecules comprise a crRNA-tacrRNA scaffold polynucleotide and a targeting sequence corresponding to a genomic target of interest.

In some embodiments, the CRISPR/Cas system recognizes a target site in a gene involved in plantlet and/or berry color determination. In some embodiments, the CRISPR/Cas system recognizes a target in one or more of a PDS1 gene, a mybA1 gene, and a Gret1 retrotransposon. The CRISPR/Cas system as described herein may bind to and/or cleave the region of interest in a region upstream of the coding region of a gene involved in plantlet and/or berry color determination. In some embodiments, the CRISPR/Cas system generates a specific sequence change in the 5′-UTR of a gene involved in plantlet and/or berry color determination, such as generating a single nucleotide gene edit to form an out-of-frame start codon upstream of the gene's ORF. In some embodiments, the gene edit (e.g., deletion, insertion, or substitution) results in production of an upstream, out-of-frame start codon that may result in the elimination of protein production or a nonfunctional protein. In some embodiments, the CRISPR/Cas system generates a specific sequence change or gene edit (e.g., insertion, deletion, or substitution) in the coding region or a non-coding region of a gene involved in plantlet and/or berry color determination, such as generating a large deletion to form (1) an out-of-frame start codon upstream of the gene's ORF, thereby suppressing expression of the gene involved in plantlet and/or berry color determination, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases.

The CRISPR/Cas system can be based on the Cas9 nuclease and an engineered single guide RNA (sgRNA) that specifies the targeted nucleic acid sequence. Cas9 is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tacrRNA).

The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can induce site-specific double strand breaks (DSBs) into genomic DNA of live cells. See, e.g., Mussolino, Nat. Biothechnol., 31:208-209 (2013). In some embodiments, the Cas9 protein is expressed in a plant cell as a fusion to a nuclear localization signal (NLS) to ensure delivery into nuclei. In some embodiments, the Cas9 protein is tagged (e.g., FLAG- or GFP-tagged). In some embodiments, promoters (e.g., EC1/EC2 promoter, CaMV 35S promoter, UBQ10 promoter, ACT2 promoter, RPS5A promoter, or DMC1 promoter) may be used to drive Cas9 expression in a plant cell. In some embodiments, the Cas9 enzyme is S. pneumoniae, S. pyogenes, or S. thermophiles Cas9, and may include mutated Cas9 derived from these organisms. The enzyme may be a Cas9 homolog or ortholog. In some embodiments, the CRISPR enzyme (e.g., Cas9 enzyme) is codon-optimized for expression in a plant cell, such as a Vitis cell.

The CRISPR/Cas system can be based on the Cpf1 nuclease and an engineered single guide RNA (sgRNA) that specifies the targeted nucleic acid sequence.

Cpf1 is distinguished from Cas9 by a its single RuvC endonuclease active site, its 5′ protospacer adjacent motif preference, and for creating sticky rather than blunt ends at the cut site. The Cpf1 protein has a RuvC-like endonuclease domain that is similar to the RuvC domain of Cas9. Cpf1 does not have a HNH endonuclease domain, and the N-terminal of Cpf1 does not have an alpha-helical recognition lobe, unlike Cas9. In some embodiments, the Cpf1 protein is tagged (e.g., FLAG- or GFP-tagged). In some embodiments, promoters (e.g., EC1/EC2 promoter, CaMV 35S promoter, UBQ10 promoter, ACT2 promoter, RPS5A promoter, or DMC1 promoter) may be used to drive Cpf1 expression in a plant cell. In some embodiments, the Cpf1 enzyme is Francisella tularensis subsp. Novicida Cpf1, and may include mutated Cpf1 derived from these organisms. The enzyme may be a Cpf1 homolog or ortholog. In some embodiments, the CRISPR enzyme (e.g., Cpf1 enzyme) is codon-optimized for expression in a plant cell, such as a Vitis cell.

The CRISPR/Cas system can be based on the Csm1 nuclease and an engineered single guide RNA (sgRNA) that specifies the targeted nucleic acid sequence.

Csm1 belongs to the Cas10 family of endonucleases. Csm1 is the largest subunit of the Csm interference complex in the type III-A CRISPR system. Csm1 exhibits ssDNA-specific endo- and exonuclease activity. In some embodiments, promoters (e.g., EC1/EC2 promoter, CaMV 35S promoter, UBQ10 promoter, ACT2 promoter, RPS5A promoter, or DMC1 promoter) may be used to drive Csm1 expression in a plant cell. In some embodiments, the Csm1 enzyme is Thermococcus onnurineus Csm1, and may include mutated Csm1 derived from these organisms. The enzyme may be a Csm1 homolog or ortholog. In some embodiments, the CRISPR enzyme (e.g., Csm1 enzyme) is codon-optimized for expression in a plant cell, such as a Vitis cell.

The single guide RNA (sgRNA) is the second component of the CRISPR/Cas system that forms a complex with a Cas nuclease. The sgRNA is created by fusing crRNA with tacrRNA. The sgRNA guide sequence located at the 5′ end confers DNA target specificity. By modifying the guide sequence, sgRNAs with different target specificities can be designed to target any desired endogenous gene. In some embodiments, the target sequence is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence is about 1 to about 10 base pairs upstream of the transcription start site (e.g., positions −10, −9, −8, −7, −6, −5, −4, −3, −2, or −1). In some embodiments, the target sequence is located within the open reading frame of the gene of interest. In some embodiments, the target sequence is located within a coding region of the gene of interest. In some embodiments, the CRISPR/Cas system comprises at least one sgRNA. In some embodiments, a target sequence of the at least one sgRNA is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence of the at least one sgRNA is about 1 to about 10 base pairs upstream of the transcription start site (e.g., positions −10, −9, −8, −7, −6, −5, −4, −3, −2, or −1). In some embodiments, the target sequence of the at least one sgRNA is located within the open reading frame of the gene of interest. In some embodiments, the target sequence of the at least one sgRNA is located within a coding region of the gene of interest. In some embodiments, the target sequences of the at least one sgRNA is located within the open reading frame of the gene of interest. In some embodiments, the target sequences of the at least one sgRNA is located within a coding region of the gene of interest. In some embodiments, the CRISPR/Cas system comprises one sgRNA, wherein the one sgRNA targets two regions of a gene having the same sequence, such as two inverted terminal repeats (ITRs). In some embodiments, the target sequences of the sgRNA are separated by at least 50 bases, at least 100 bases, at least 200 bases, at least 500 bases, at least 1000 bases, at least 2000 bases, at least 5000 bases, or at least 10000 bases.

In some embodiments, the CRISPR/Cas system comprises at least two sgRNAs. In some embodiments, a target sequence of at least one of the at least two sgRNAs is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence of at least one of the at least two sgRNAs is about 1 to about 10 base pairs upstream of the transcription start site (e.g., positions −10, −9, −8, −7, −6, −5, −4, −3, −2, or −1). In some embodiments, the target sequence of at least one of the at least two sgRNAs is located within the open reading frame of the gene of interest. In some embodiments, the target sequence of at least one of the at least two sgRNAs is located within a coding region of the gene of interest. In some embodiments, the target sequences of at least two of the at least two sgRNAs are located within the open reading frame of the gene of interest. In some embodiments, the target sequences of at least two of the at least two sgRNAs are located within a coding region of the gene of interest. In some embodiments, the CRISPR/Cas system comprises two sgRNAs, wherein the two sgRNAs have non-overlapping target sequences. In some embodiments, the target sequences of the two sgRNAs are separated by at least 50 bases, at least 100 bases, at least 200 bases, at least 500 bases, at least 1000 bases, at least 2000 bases, at least 5000 bases, or at least 10000 bases. In some embodiments, a gRNA comprises the nucleic acid sequence set forth in SEQ ID NO: 26. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 26. In some embodiments, a gRNA comprises the nucleic acid sequence set forth in SEQ ID NO: 27. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 27. In some embodiments, a gRNA comprises the nucleic acid sequence set forth in SEQ ID NO: 28. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 28. In some embodiments, a gRNA comprises the nucleic acid sequence set forth in SEQ ID NO: 29. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 29. In some embodiments, a gRNA comprises the nucleic acid sequence set forth in SEQ ID NO: 30. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 30. In some embodiments, a gRNA comprises the nucleic acid sequence set forth in SEQ ID NO: 31. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 31. In some embodiments, a gRNA comprises the nucleic acid sequence set forth in SEQ ID NO: 32. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 32. In some embodiments, a gRNA comprises the nucleic acid sequence set forth in SEQ ID NO: 33. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 33. In some embodiments, a gRNA comprises the nucleic acid sequence set forth in SEQ ID NO: 34. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 34. In some embodiments, a gRNA comprises the nucleic acid sequence set forth in SEQ ID NO: 35. In some embodiments, a gRNA comprises a nucleic acid sequence having at least 80%, at least 85%, at least 90%, at least 95%, at least 96%, at least 97%, at least 98%, or at least 99% sequence identity to SEQ ID NO: 35.

It is not intended that the present technology be limited to any particular distance restraint with regard to the location of the guide RNA target sequence from the gene transcription start site. In some embodiments, the target sequence lies “in proximity to” a gene of interest, where “in proximity to” refers to any distance from the gene of interest, wherein the Cas-regulatory domain fusion is able to exert an effect on gene expression. In some embodiments, the target sequence lies upstream of the ORF of the gene of interest.

The canonical length of the guide sequence is about 20 bp and the DNA target sequence is about 20 bp followed by a PAM sequence having the consensus NGG sequence. In some embodiments, sgRNAs are expressed in a plant cell using plant RNA polymerase III promoters, such as U6 and U3.

When the DSBs are repaired by either NHEJ or HDR, the sequence at the repair site can be modified or new genetic information can be inserted (e.g., donor DNA comprising a desired gene edit can be inserted into the target gene at the break site). Although HDR typically occurs at lower and more variable frequencies than NHEJ, it can be leveraged to generate precise, defined modifications at a target locus in the presence of an exogenously introduced repair template. Accordingly, exogenous repair templates, designed by methods known in the art, can also be delivered into a cell, most often in the form of a synthetic, single-stranded DNA donor oligo or DNA donor plasmid, to generate a precise change in the genome. Single-stranded DNA donor oligos are delivered into a cell to insert or change short sequences (SNPs, amino acid substitutions, epitope tags, etc.) of DNA in the endogenous genomic target region. The benefits of using a synthetic DNA donor oligo is that no cloning is required to generate the donor template and DNA modifications can be added during synthesis for different applications, such as increased resistance to nucleases. Traditionally, the maximum insert length recommended for use with a DNA donor oligo is about 50 nucleotides.

In some embodiments, the present technology provides an engineered, programmable, non-naturally occurring CRISPR/Cas system comprising a Cas9 protein and one or more single guide RNAs (sgRNAs) that target the genomic loci of DNA molecules encoding one or more gene products associated with plant color or plant part color, and the Cas9 protein cleaves the genomic loci of the DNA molecules encoding the one or more gene products, whereby expression of the one or more gene products is altered. In some embodiments, Cas9 introduces multiple DSBs in the same cell (i.e., multiplexes) via expression of one or more distinct guide RNAs.

In some embodiments, the present technology provides a method for targeted genomic modification of plant cells to alter the expression of at least one gene involved in plantlet and/or berry color determination, the method comprising introducing into a plant cell, comprising and expressing a DNA molecule having a target sequence and encoding the gene involved in plantlet and/or berry color determination, an engineered CRISPR/Cas system comprising (a) an expression construct comprising a first polynucleotide encoding a Cas9 protein, or a variant thereof or a fusion protein therewith, and a second polynucleotide encoding a guide RNA comprising: (i) a crRNA-tracrRNA scaffold polynucleotide, and (ii) a targeting sequence operably linked to the crRNA-tracrRNA scaffold polynucleotide, where the targeting sequence corresponds to a genomic locus of interest, and (b) delivering the expression construct into the plant cell, where the first and second polynucleotides are expressed (transcribed) within the plant cell. This method can optionally further include visualizing, identifying, or selecting for plant cells having a genomic modification at the genomic locus of interest that is induced by the delivering the expression construct into the plant cell.

In some embodiments of the methods of the present technology, the Cas9 polypeptide and one or more guide RNA are encoded on a single vector. In some embodiments, the single vector is a plasmid. In some embodiments of the methods of the present technology, the Cas9 polypeptide and the one or more guide RNA are encoded on two separate vectors. In these methods, the steps generally follow the sequence of introducing into a plant cell containing and expressing a DNA molecule having a target sequence and encoding the gene involved in plantlet and/or berry color determination an engineered CRISPR/Cas system comprising (a) a Cas9 polynucleotide or a conservative variant thereof, and a guide RNA comprising (i) a crRNA-tracrRNA scaffold polynucleotide, and (ii) a targeting sequence operably linked to the crRNA-tracrRNA scaffold polynucleotide, with the targeting sequence corresponding to a genomic locus of interest, and (b) delivering the two polynucleotides into the plant cell. In variations of this method, a donor polynucleotide having homology to the genomic target of interest is included in a co-transfection. In some variations of these methods, the transfected material can be either plasmid DNA or RNA generated by in vitro transcription. In still other variations, the methods for targeted genomic modification are multiplexed, meaning that more than one genomic locus is targeted for modification. In still other variations of these methods, the transformation of the plant cells can be followed by visualizing, identifying, or selecting for plant cells having a genomic modification at the genomic locus of interest.

### Meganucleases

In some embodiments, the compositions and methods described herein employ a meganuclease DNA binding domain for binding to a region of interest in the genome of a plant cell. Meganucleases are engineered versions of naturally occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG family (“LAGLIDADG” is disclosed as SEQ ID NO: 21), the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-CeuI, PI-PspI, PI-Sce, I-SceIV, I-CsmI, I-PanI, I-SceII, I-PpoI, I-SceIII, I-CreI, I-TevI, I-TevII, and I-TevIII are known.

In some embodiments, the meganuclease is tailored to recognize a target in one or more of a PDS1 gene, a mybA1 gene, and a Gret1 retrotransposon. The meganucleases as described herein may bind to and/or cleave the region of interest in a region upstream of the coding region of a gene involved in plantlet and/or berry color determination. Gene insertion or correction can be achieved by the introduction of a DNA repair matrix containing sequences homologous to the endogenous sequence surrounding the DNA break. Gene edits can be created either at or distal to the break. In some embodiments, the meganuclease generates a specific sequence change in the 5′-UTR of a gene involved in plantlet and/or berry color determination, such as generating a single nucleotide gene edit to form an out-of-frame start codon upstream of the gene's ORF.

### TALENs

In some embodiments, the compositions and methods described herein employ transcription activator-like effector nucleases (TALENs) to edit plant genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, after a target sequence in a gene involved in plantlet and/or berry color determination is identified, a corresponding TALEN sequence is engineered and inserted into a plasmid. The plasmid is inserted into a target cell where it is translated to produce a functional TALEN, which then enters the nucleus where it binds to and cleaves its target sequence. Such an approach can be employed to introduce an exogenous DNA sequence into the target gene as the DSB is being repaired through either homology-directed repair or non-homologous end-joining. For example, in some embodiments, the use of TALEN technology generates a specific sequence change (e.g., insertion, deletion, or substitution) in the 5′-UTR of a gene involved in plantlet and/or berry color determination, resulting in the production of an out-of-frame start codon upstream of the gene's ORF.

### ZFNs

In some embodiments, the compositions and methods described herein employ zinc finger nucleases (ZFNs) to edit plant genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the FokI restriction enzyme responsible for cleaving DNA. ZFNs can be engineered according to methods known in the art to bind to a desired DNA sequence and cleave DNA at specific locations. For example, after a target sequence in a gene involved in plantlet and/or berry color determination is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in the 5′-UTR of a gene involved in plantlet and/or berry color determination, such as the insertion of an out-of-frame start codon upstream of the gene's ORF.

### Quantifying Color Change

Methods of ascertaining color change of a plant or a berry produced by a plant are available to those skilled in the art. In some embodiments of the present technology, genetically engineered plants and cells are characterized by altered color change of one or more components, such as a plantlet or a berry. In some embodiments, an edited genome results in a change in the color of a plantlet and/or a plantlet produced by a plant derived from a plant cell. In some embodiments, an edited genome results in a change in the color of a fully-mature fruit (e.g., grape) produced by a plant derived from a plant cell.

In some embodiments, an edited genome results in a change in the color of a plantlet produced by a plant derived from a plant cell from green to white. In other words, an edited genome in a plant cell giving rise to a plant can result in a plant having a white plantlet, whereas a corresponding plant that does not comprise the edited genome has a green plantlet.

In some embodiments, an edit in a PDS1 gene results in a change in the color of a plantlet produced by a plant derived from a plant cell from green to white. In other words, an edit in a PDS1 gene in a plant cell giving rise to a plant can result in a plant having a white plantlet, whereas a corresponding plant that does not comprise the edited genome has a green plantlet. In some embodiments, the plant cell is a Vitis vinifera cell. In some embodiments, the plant cell is a Vitis vinifera ‘Thompson Seedless’ (TS) cell.

In some embodiments, an edited genome results in a change in the color of a plantlet produced by a plant derived from a plant cell from red to green. In other words, an edited genome in a plant cell giving rise to a plant can result in a plant having a green plantlet, whereas a corresponding plant that does not comprise the edited genome has a red plantlet.

In some embodiments, an edit in a mybA1 gene results in a change in the color of a plantlet produced by a plant derived from a plant cell from red to green. In other words, an edit in a mybA1 gene in a plant cell giving rise to a plant can result in a plant having a green plantlet, whereas a corresponding plant that does not comprise the edited genome has a red plantlet. In some embodiments, the plant cell is a Vitis vinifera cell. In some embodiments, the plant cell is a Vitis vinifera Anthocyanin overexpressed ‘Thompson Seedless’ (AOTS) cell. In some embodiments, an edited genome results in a change in the color of a fully-mature fruit (e.g., grape) produced by a plant derived from a plant cell from dark purple to green. In other words, an edited genome in a plant cell giving rise to a plant can result in a plant having a green fully-mature fruit (e.g., grape), whereas a corresponding plant that does not comprise the edited genome has a dark purple fully-mature fruit (e.g., grape). In some preferred embodiments, the fully-mature fruit is a grape.

In some embodiments, an edit in a mybA1 gene results in a change in the color of a fully-mature fruit (e.g., grape) produced by a plant derived from a plant cell from dark purple to green. In other words, an edit in a mybA1 gene in a plant cell giving rise to a plant can result in a plant having a green fully-mature fruit (e.g., grape), whereas a corresponding plant that does not comprise the edited genome has a dark purple fully-mature fruit (e.g., grape). In some embodiments, the plant cell is a Vitis vinifera cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to red. In other words, an edit in a Gret1 sequence in a plant cell giving rise to a plant can result in a plant having a red fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to light red. In other words, an edit in a Gret1 sequence in a plant cell giving rise to a plant can result in a plant having a light red fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to dark red. In other words, an edit in a Gret1 sequence in a plant cell giving rise to a plant can result in a plant having a dark red fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to purple. In other words, an edit in a Gret1 sequence in a plant cell giving rise to a plant can result in a plant having a purple fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to orange. In other words, an edit in a Gret1 sequence in a plant cell giving rise to a plant can result in a plant having an orange fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

In some embodiments, an edit in a Gret1 sequence results in a change in the color of a fully-mature fruit (e.g., grape) produced by a plant derived from a plant cell from green to pink. In other words, an edit in a Gret1 sequence in a plant cell giving rise to a plant can result in a plant having a pink fully-mature fruit (e.g., grape), whereas a corresponding plant that does not comprise the edited genome has a green fully-mature fruit (e.g., grape). In some embodiments, the plant cell is a Vitis vinifera cell. In some embodiments, the plant cell is an Italia cell. In some embodiments, the plant cell is a Muscat of Alexandria cell. In some embodiments, the plant cell is a SUGRA35 cell. In some embodiments, the plant cell is a Vitis vinifera ‘Thompson Seedless’ (TS) cell. In some embodiments, the plant cell is a Vitis vinifera ‘Merlot’ cell.

### Host Plants and Cells

In some embodiments, the present technology relates to the genetic manipulation of a plant or cell via targeted genome engineering (also known as genome editing) techniques that can be used to generate edits in genes of interest to genetically engineer plantlet and/or fruit color. In some embodiments, without wishing to be bound by theory, the introduction of a large deletion can inactivate or attenuate a gene involved in plantlet and/or fruit color determination. Accordingly, the present technology provides methodology and constructs for altering the color of a plantlet and/or a fruit in a plant.

Plants for use in the methods of the present technology are species of Vitis, such as Vitis vinifera. Any strain or variety of Vitis may be used. In some embodiments, strains that already contain altered gene expression related to plantlet and/or fruit or berry color are used in the methods of the present technology.

Any plant tissue capable of subsequent clonal propagation, whether by organogenesis or embryogenesis, may be transformed with a vector of the present technology. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, callus tissue, existing meristematic tissue (e.g., apical meristems, axillary buds, and root meristems), and induced meristem tissue (e.g., cotyledon meristem and hypocotyl meristem).

Plants of the present technology may take a variety of forms. The plants may be chimeras of transformed cells and non-transformed cells; the plants may be clonal transformants (e.g., all cells transformed to contain the transcription cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npill) can be associated with the transcription cassette to assist in breeding.

In view of the foregoing, it will be apparent that plants that may be employed in practicing the present technology include those of the genus Vitis.

Methods of making engineered plants of the present technology, in general, involve first providing a plant cell capable of regeneration. The plant cell is then transformed with a nucleic acid construct/expression vector or other nucleic acids (such as RNA) of the present technology and an engineered plant is regenerated from the transformed plant cell. Any of the nucleic acid constructs used for reducing the expression of a color or pigment-associated gene can be delivered in vivo or ex vivo by any suitable means known in the art including, but not limited to, electroporation, viral transduction, viral vectors, and lentiviral vectors. In plants, expression systems have been employed to implement the CRISPR/Cas9 system. Widely used assays in plant research include protoplast transformation, the floral dip method, and leaf tissue transformation using the agroinfiltration method (also known as the Agrobacterium tumefaciens-mediated transient expression assay). See, e.g., Belhaj et al., Plant Methods, 9:39 (2013). The agroinfiltration method, which is performed on intact plants, is based on infiltration of Agrobacterium tumefaciens strains carrying a binary plasmid that contains the candidate genes to be expressed. Transgenic plants can be easily regenerated out of agroinfiltrated tissue and can be used to generate plants carrying the specified gene edits. See, e.g., Nekrasov et al., Nat. Biotechnol., 31:691-693 (2013). Numerous Agrobacterium vector systems useful in methods of the present technology are known. For example, U.S. Pat. No. 4,459,355 discloses a method for transforming susceptible plants, including dicots, with an Agrobacterium strain containing the Ti plasmid. The transformation of woody plants with an Agrobacterium vector is disclosed in U.S. Pat. No. 4,795,855. Further, U.S. Pat. No. 4,940,838 discloses a binary Agrobacterium vector (i.e., one in which the Agrobacterium contains one plasmid having the vir region of a Ti plasmid but no T region, and a second plasmid having a T region but no vir region) useful in carrying out the present technology. The aforementioned methods of delivering nucleases and/or donor constructs are well known to those skilled in the art and any of the methods can be used to produce a Vitis plant having altered expression of color or pigment-associated genes, and thus altered color relative to a non-transformed control plant of the same strain.

After transformation of the plant cells or plant, those plant cells or plants into which the desired DNA has been incorporated may be selected by methods known in the art, including but not limited to the restriction enzyme site loss assay and the Surveyor assay. See, e.g., Belhaj et al. (2013).

Various assays may be used to determine whether the plant cell shows a change in gene expression, for example, Northern blotting or quantitative reverse transcriptase PCR (RT-PCR).

Products of Plants with Altered Plantlet and/or Fruit Color

The methods of the present technology provide genetically-engineered cells and plants having altered plantlet color and/or fruit color compared to non-genetically-engineered cells and plants of the same strain. For example, the present technology contemplates changing plantlet and/or fruit color through the use of targeted genome engineering techniques to generate edits resulting in large deletions in color-associated genes and/or genetic elements (e.g., PDS1 gene, mybA1 gene, or Gret1 retrotransposon), thereby suppressing protein expression in the transformed cell or plant.

Vitis plants according to the present technology with reduced expression of one or more genes involved in plantlet and/or fruit color determination described herein will be desirable in the production of certain products.

### Embodiments

Embodiment 1: A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least two guide RNAs (gRNA), wherein one of the two gRNAs comprises a nucleotide sequence set forth in SEQ ID NO: 2, and wherein one of the two gRNAs comprises a nucleotide sequence set forth in SEQ ID NO: 3, or at least one polynucleotide encoding the at least two gRNAs, and
(b) an effector protein, or one or more polynucleotides encoding the effector protein;
wherein each of the at least two gRNAs hybridize to a PDS1 gene sequence, and each of the at least two gRNAs form a complex with the effector protein; and
wherein the effector protein is a Cas protein comprising a nuclease and/or an effector domain.

Embodiment 2: The method of embodiment 1, wherein the Cas protein is a Cas9 protein, a Cpf1 protein, or a Csm1 protein.

Embodiment 3: The method of embodiment 1 or 2, wherein the edited genome results in a change in the color of a plantlet produced by a plant derived from the plant cell from green to white.

Embodiment 4: The method of embodiment 1 or 2, wherein a plantlet produced by a plant derived from the plant cell comprising the edited genome is white, and wherein a plantlet produced by a plant derived from a plant cell that does not comprise the edited genome is green.

Embodiment 5: The method of any one of embodiments 1-4, wherein the plant cell is a Vitis vinifera ‘Thompson Seedless’ (TS) cell.

Embodiment 6: A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least two guide RNAs (gRNA), wherein one of the two gRNAs comprises a nucleotide sequence set forth in SEQ ID NO: 19, and wherein one of the two gRNAs comprises a nucleotide sequence set forth in SEQ ID NO: 20, or at least one polynucleotide encoding the at least two gRNAs, and
(b) an effector protein, or one or more polynucleotides encoding the effector protein;
wherein each of the at least two gRNAs hybridize to a mybA1 gene sequence, and each of the at least two gRNAs form a complex with the effector protein; and
wherein the effector protein is a Cas protein comprising a nuclease and/or an effector domain.

Embodiment 7: The method of embodiment 6, wherein the Cas protein is a Cas9 protein, a Cpf1 protein, or a Csm1 protein.

Embodiment 8: The method of embodiment 6 or 7, wherein the edited genome results in a change in the color of a plantlet produced by a plant derived from the plant cell from red to green.

Embodiment 9: The method of embodiment 6 or 7, wherein a plantlet produced by a plant derived from the plant cell comprising the edited genome is green, and wherein a plantlet produced by a plant derived from a plant cell that does not comprise the edited genome is red.

Embodiment 10: The method of any one of embodiments 6-9, wherein the plant cell is a Vitis vinifera Anthocyanin overexpressed ‘Thompson Seedless’ (AOTS) cell.

Embodiment 11: The method of any one of embodiments 6-9, wherein the plant cell is a Vitis vinifera ‘Merlot’ cell.

Embodiment 12: The method of embodiment 11, wherein the edited genome results in a change in the color of a fully-mature fruit produced by a plant derived from the plant cell from dark purple to green.

Embodiment 13: The method of embodiment 11, wherein a mature fruit produced by a plant derived from the plant cell comprising the edited genome is green, and wherein a mature fruit produced by a plant derived from a plant cell that does not comprise the edited genome is dark purple.

Embodiment 14: The method of embodiment 11, wherein a fruit produced by a plant derived from the plant cell comprising the edited genome maintains a green color throughout the course development, and wherein a fruit produced by a plant derived from a plant cell that does not comprise the edited genome changes from green to dark purple throughout the course of development.

Embodiment 15: A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least one guide RNA (gRNA) which comprises a guide sequence capable of hybridizing with a PDS1 gene sequence, or at least one polynucleotide encoding the at least one gRNA, and
(b) an effector protein, or one or more polynucleotides encoding the effector protein;
wherein the at least one gRNA hybridizes to the PDS1 gene sequence, and the at least one gRNA forms a complex with the effector protein; and
wherein the effector protein is a Cas protein comprising a nuclease and/or an effector domain.

Embodiment 16: The method of embodiment 15, wherein the at least one gRNA comprises a nucleotide sequence set forth in SEQ ID NO: 2 or SEQ ID NO: 3.

Embodiment 17: The method of embodiment 15 or 16, wherein the CRISPR-Cas system comprises two gRNAs or at least one polynucleotide encoding the two gRNAs.

Embodiment 18: The method of embodiment 17, wherein one of the two gRNAs comprises a nucleotide sequence set forth in SEQ ID NO: 2, and wherein one of the two gRNAs comprises a nucleotide sequence set forth in SEQ ID NO: 3.

Embodiment 19: The method of any one of embodiments 15-18, wherein the edited genome results in a change in the color of a plantlet produced by a plant derived from the plant cell from green to white.

Embodiment 20: The method of any one of embodiments 15-18, wherein a plantlet produced by a plant derived from the plant cell comprising the edited genome is white, and wherein a plantlet produced by a plant derived from a plant cell that does not comprise the edited genome is green.

Embodiment 21: The method of any one of embodiments 15-20, wherein the Cas protein is a Cas9 protein, a Cpf1 protein, or a Csm1 protein.

Embodiment 22: The method of any one of embodiments 15-21, wherein the plant cell is a Vitis vinifera ‘Thompson Seedless’ (TS) cell.

Embodiment 23: A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least one guide RNA (gRNA) which comprises a guide sequence capable of hybridizing with a mybA1 gene sequence, or at least one polynucleotide encoding the at least one gRNA, and
(b) an effector protein, or one or more polynucleotides encoding the effector protein;
wherein the at least one gRNA hybridizes to the mybA1 gene sequence, and the at least one gRNA forms a complex with the effector protein; and
wherein the effector protein is a Cas protein comprising a nuclease and/or an effector domain.

Embodiment 24: The method of embodiment 23, wherein the at least one gRNA comprises a nucleotide sequence set forth in SEQ ID NO: 19 or SEQ ID NO: 20.

Embodiment 25: The method of embodiment 23 or 24, wherein the CRISPR-Cas system comprises two gRNAs or at least one polynucleotide encoding the two gRNAs.

Embodiment 26: The method of embodiment 25, wherein one of the two gRNAs comprises a nucleotide sequence set forth in SEQ ID NO: 19, and wherein one of the two gRNAs comprises a nucleotide sequence set forth in SEQ ID NO: 20.

Embodiment 27: The method of any one of embodiments 23-26, wherein the edited genome results in a change in the color of a plantlet produced by a plant derived from the plant cell from red to green.

Embodiment 28: The method of any one of embodiments 23-26, wherein a plantlet produced by a plant derived from the plant cell comprising the edited genome is green, and wherein a plantlet produced by a plant derived from a plant cell that does not comprise the edited genome is red.

Embodiment 29: The method of any one of embodiments 23-28, wherein the plant cell is a Vitis vinifera Anthocyanin overexpressed ‘Thompson Seedless’ (AOTS) cell.

Embodiment 30: The method of any one of embodiments 23-29, wherein the plant cell is a Vitis vinifera ‘Merlot’ cell.

Embodiment 31: The method of embodiment 30, wherein the edited genome results in a change in the color of a fruit produced by a plant derived from the plant cell from dark purple to green.

Embodiment 32: The method of any one of embodiments 23-31, wherein the Cas protein is a Cas9 protein, a Cpf1 protein, or a Csm1 protein.

Embodiment 33: A method for producing an edited genome in a plant cell, the method comprising introducing into the cell a gene editing system comprising at least one exogenous nuclease, or one or more polynucleotides encoding the gene editing system, wherein the nuclease cleaves endogenous genomic sequences in the cell, wherein the cell is a Vitis cell.

Embodiment 34: The method of embodiment 33, wherein the nuclease is selected from the group consisting of a CRISPR associated (Cas) nuclease, a meganuclease, a zinc finger protein nuclease (ZFN), a transcription activator-like effector nuclease (TALEN), and combinations thereof.

Embodiment 35: The method of embodiment 33 or 34, wherein the edited genome comprises an insertion, deletion, or substitution resulting in an upstream, out-of-frame start codon in a grape-pigment-associated gene, thereby decreasing expression of a gene product of the grape-pigment-associated gene relative to a control cell.

Embodiment 36: The method of any one of embodiments 33-35, wherein the edited genome results in a change in the color of a plantlet and/or a fruit produced by a plant derived from the cell, relative to a plantlet and/or a fruit produced by a plant derived from a cell of the same species in which the edited genome was not produced.

Embodiment 37: The method of any one of embodiments 33-36, wherein the Vitis cell is a Vitis vinifera ‘Thompson Seedless’ cell (TS).

Embodiment 38: The method of any one of embodiments 33-37, wherein the grape-pigment-associated gene is PDS1.

Embodiment 39: The method of any one of embodiments 33-38, wherein the edited genome results in a change in the color of a plantlet produced by a plant derived from the plant cell from green to white.

Embodiment 40: The method of any one of embodiments 33-38, wherein a plantlet produced by a plant derived from the plant cell comprising the edited genome is white, and wherein a plantlet produced by a plant derived from a plant cell that does not comprise the edited genome is green.

Embodiment 41: The method of any one of embodiments 33-40, wherein the gene editing system comprises:
(a) at least one guide RNA (gRNA) which comprises a guide sequence capable of hybridizing with a target sequence, or at least one polynucleotide encoding the at least one gRNA, and (b) an effector protein, or one or more polynucleotides encoding the effector protein;
wherein the at least one gRNA hybridizes to the target sequence, and the at least one gRNA forms a complex with the effector protein; and
wherein the effector protein comprises a nuclease and/or an effector domain.

Embodiment 42: The method of embodiment 41, wherein the at least one gRNA is capable of hybridizing to a PDS1 gene.

Embodiment 43: The method of embodiment 41 or 42, wherein the at least one gRNA comprises a nucleotide sequence set forth in SEQ ID NO: 2 or SEQ ID NO: 3.

Embodiment 44: The method of any one of embodiments 41-43, wherein the gene editing system comprises two gRNAs or at least one polynucleotide encoding the two gRNAs.

Embodiment 45: The method of embodiment 44, wherein one of the two gRNAs comprises a nucleotide sequence set forth in SEQ ID NO: 2, and wherein one of the two gRNAs comprises a nucleotide sequence set forth in SEQ ID NO: 3.

Embodiment 46: The method of any one of embodiments 33-36, wherein the Vitis cell is a Vitis vinifera Anthocyanin-over-expressed ‘Thompson Seedless’ cell (AOTS) or a Vitis vinifera ‘Merlot’ cell.

Embodiment 47: The method of embodiment 46, wherein the Vitis cell is an AOTS cell.

Embodiment 48: The method of embodiment 47, wherein the grape-pigment-associated gene is mybA1.

Embodiment 49: The method of any one of embodiments 33-36 and 46-48, wherein the edited genome results in a change in the color of a plantlet produced by a plant derived from the plant cell from red to green.

Embodiment 50: The method of any one of embodiments 33-36 and 46-48, wherein a plantlet produced by a plant derived from the plant cell comprising the edited genome is green, and wherein a plantlet produced by a plant derived from a plant cell that does not comprise the edited genome is red.

Embodiment 51: The method of any one of embodiments 46-50, wherein the gene editing system comprises:
(a) at least one guide RNA (gRNA) which comprises a guide sequence capable of hybridizing with a target sequence, or at least one polynucleotide encoding the at least one gRNA, and
(b) an effector protein, or one or more polynucleotides encoding the effector protein;
wherein the at least one gRNA hybridizes to the target sequence, and the at least one gRNA forms a complex with the effector protein; and
wherein the effector protein comprises a nuclease and/or an effector domain.

Embodiment 52: The method of embodiment 51, wherein the at least one gRNA is capable of hybridizing to a mybA1 gene.

Embodiment 53: The method of embodiment 51 or 52, wherein the at least one gRNA comprises a nucleotide sequence set forth in SEQ ID NO: 19 or SEQ ID NO: 20.

Embodiment 54: The method of any one of embodiments 51-53, wherein the gene editing system comprises two gRNAs or at least one polynucleotide encoding the two gRNAs.

Embodiment 55: The method of embodiment 54, wherein one of the two gRNAs comprises a nucleotide sequence set forth in SEQ ID NO: 19, and wherein one of the two gRNAs comprises a nucleotide sequence set forth in SEQ ID NO: 20.

Embodiment 56: The method of embodiment 46, wherein the Vitis cell is a ‘Merlot’ cell.

Embodiment 57: The method of embodiment 56, wherein the grape-pigment-associated gene is mybA1.

Embodiment 58: The method of embodiment 56 or 57, wherein the edited genome results in a change in the color of a fruit produced by a plant derived from the plant cell from dark purple to green.

Embodiment 59: The method of any one of embodiments 56-58, wherein a mature fruit produced by a plant derived from the plant cell comprising the edited genome is green, and wherein a mature fruit produced by a plant derived from a plant cell that does not comprise the edited genome is dark purple.

Embodiment 60: The method of any one of embodiments 56-58, wherein a fruit produced by a plant derived from the plant cell comprising the edited genome maintains a green color throughout the course development, and wherein a fruit produced by a plant derived from a plant cell that does not comprise the edited genome changes from green to dark purple throughout the course of development.

Embodiment 61: The method of any one of embodiments 33-60, wherein the gene editing system is a Clustered Regularly Interspaced Short Palindromic Repeats (CRISPR)-associated (CRISPR-Cas) system.

Embodiment 62: The method of any one of embodiments 33-61, wherein the nuclease is a Cas9 enzyme, a Cpf1 enzyme, or a Csm1 enzyme.

Embodiment 63: A genetically engineered Vitis cell produced by the method of any one of embodiments 1-62.

Embodiment 64: The genetically engineered Vitis cell of embodiment 63, wherein a plant derived from the cell produces a plantlet and/or a fruit that has altered pigmentation relative to a plantlet and/or a fruit produced by a plant derived from a cell of the same strain that was not produced by the method.

Embodiment 65: A genetically engineered Vitis plant produced by the method of any one of embodiments 1-62, or derived from the genetically engineered Vitis cell of embodiment 63 or 64.

Embodiment 66: A product comprising the genetically engineered Vitis plant of embodiment 65, or portions thereof, wherein the product has altered pigmentation relative to a product produced by a non-genetically engineered plant of the same strain.

Embodiment 67: The product of embodiment 66, wherein the product comprises a callus.

Embodiment 68: The product of embodiment 66 or 67, wherein the product comprises a plantlet.

Embodiment 69: The product of any one of embodiments 66-68, wherein the product comprises a fruit.

Embodiment 70: The product of any one of embodiments 66-69, wherein the product comprises a grape.

Embodiment 71: The product of any one of embodiments 66-70, wherein the product is a wine.

Embodiment 72: A method for reducing expression of at least one grape-pigment-associated gene product in a Vitis cell comprising introducing into the cell, comprising and expressing a DNA molecule having a target sequence and encoding the gene product, an engineered CRISPR-Cas system comprising one or more vectors comprising:
(a) a first regulatory element operable in a Vitis cell operably linked to at least one nucleotide sequence encoding at least one CRISPR-Cas system guide RNA (gRNA) that hybridizes with the target sequence, and
(b) a second regulatory element operable in a Vitis cell operably linked to a nucleotide sequence encoding a Cas9 protein, and wherein:
(i) components (a) and (b) are located on the same or different vectors of the system, (ii) the at least one gRNA targets the target sequence and the Cas9 protein cleaves the DNA molecule, and
(iii) expression of at least one gene product is reduced relative to a control cell.

Embodiment 73: The method of embodiment 72, wherein the Vitis cell is a Vitis vinifera ‘Thompson Seedless’ (TS) cell, a Vitis vinifera ‘Anthocyanin Overexpressed Thompson Seedless’ (AOTS) cell, or a Vitis vinifera ‘Merlot’ cell.

Embodiment 74: The method of embodiment 72 or 73, wherein the grape-pigment-associated gene is PDS1.

Embodiment 75: The method of any one of embodiments 72-74, wherein reducing expression results in a change in the color of a plantlet and/or a fruit produced by a plant derived from the plant cell from green to white.

Embodiment 76: The method of any one of embodiments 72-74, wherein a plantlet produced by a plant derived from the plant cell comprising the edited genome is white, and wherein a plantlet produced by a plant derived from a plant cell that does not comprise the edited genome is green.

Embodiment 77: The method of any one of embodiments 72-76, wherein the at least one gRNA is capable of hybridizing to a PDS1 gene.

Embodiment 78: The method of any one of embodiments 72-77, wherein the at least one gRNA comprises a nucleotide sequence set forth in SEQ ID NO: 2 or SEQ ID NO: 3.

Embodiment 79: The method of any one of embodiments 72-78, wherein the engineered CRISPR-Cas system comprises two gRNAs or at least one polynucleotide encoding the two gRNAs.

Embodiment 80: The method of embodiment 79, wherein one of the two gRNAs comprises a nucleotide sequence set forth in SEQ ID NO: 2, and wherein one of the two gRNAs comprises a nucleotide sequence set forth in SEQ ID NO: 3.

Embodiment 81: The method of embodiment 73, wherein the Vitis cell is a Vitis vinifera Anthocyanin-over-expressed ‘Thompson Seedless’ cell (AOTS) or a Vitis vinifera ‘Merlot’ cell.

Embodiment 82: The method of embodiment 81, wherein the Vitis cell is an AOTS cell.

Embodiment 83: The method of embodiment 81 or 82, wherein the grape-pigment-associated gene is mybA1.

Embodiment 84: The method of any one of embodiments 81-83, wherein reducing expression results in a change in the color of a plantlet and/or a fruit produced by a plant derived from the plant cell from red to green.

Embodiment 85: The method of any one of embodiments 81-83, wherein a plantlet produced by a plant derived from the plant cell comprising the edited genome is green, and wherein a plantlet produced by a plant derived from a plant cell that does not comprise the edited genome is red.

Embodiment 86: The method of any one of embodiments 81-85, wherein the at least one gRNA is capable of hybridizing to a mybA1 gene.

Embodiment 87: The method of any one of embodiments 81-86, wherein the at least one gRNA comprises a nucleotide sequence set forth in SEQ ID NO: 19 or SEQ ID NO: 20.

Embodiment 88: The method of any one of embodiments 81-87, wherein the gene editing system comprises two gRNAs or at least one polynucleotide encoding the two gRNAs.

Embodiment 89: The method of embodiment 88, wherein one of the two gRNAs comprises a nucleotide sequence set forth in SEQ ID NO: 19, and wherein one of the two gRNAs comprises a nucleotide sequence set forth in SEQ ID NO: 20.

Embodiment 90: The method of any one of embodiments 81-89, wherein the Vitis cell is a Vitis vinifera ‘Merlot’ cell.

Embodiment 91: The method of embodiment 90, wherein the grape-pigment-associated gene is mybA1.

Embodiment 92: The method of embodiment 90 or 91, wherein the edited genome results in a change in the color of a fruit produced by a plant derived from the Vitis cell from dark purple to green.

Embodiment 93: The method of any one of embodiments 90-92, wherein a mature fruit produced by a plant derived from the plant cell comprising the edited genome is green, and wherein a mature fruit produced by a plant derived from a plant cell that does not comprise the edited genome is dark purple.

Embodiment 94: The method of any one of embodiments 90-92, wherein a fruit produced by a plant derived from the plant cell comprising the edited genome maintains a green color throughout the course development, and wherein a fruit produced by a plant derived from a plant cell that does not comprise the edited genome changes from green to dark purple throughout the course of development.

Embodiment 95: The method of any one of embodiments 72-94, wherein the Cas9 protein is optimized for expression in the Vitis cell.

Embodiment 96: A genetically engineered Vitis cell produced by the method of any one of embodiments 72-95.

Embodiment 97: A genetically engineered Vitis plant comprising the cells produced by the method of any one of embodiments 72-95.

Embodiment 98: A product comprising the genetically engineered plant of embodiment 96 or 97, or portions thereof, wherein the product has altered pigmentation relative to a product produced by a non-genetically engineered plant of the same strain.

Embodiment 99: The product of embodiment 98, wherein the product comprises a callus.

Embodiment 100: The product of embodiment 98 or 99, wherein the product comprises a plantlet.

Embodiment 101: The product of any one of embodiments 98-100, wherein the product comprises a fruit.

Embodiment 102: The product of any one of embodiments 98-101, wherein the product comprises a grape.

Embodiment 103: The product of any one of embodiments 98-102, wherein the product is a wine.

Embodiment 104. A guide RNA (gRNA) comprising the nucleic acid sequence set forth in SEQ ID NO: 2 or SEQ ID NO: 3.

Embodiment 105. A guide RNA (gRNA) comprising the nucleic acid sequence set forth in SEQ ID NO: 19 or SEQ ID NO: 20.

Embodiment 106. A composition comprising:
(a) a polynucleotide comprising a sequence encoding a guide RNA (gRNA) having a nucleic acid sequence set forth in SEQ ID NO: 2 or SEQ ID NO: 3; and
(b) a polynucleotide comprising a sequence encoding a Cas enzyme.

Embodiment 107. A composition comprising: (a) a polynucleotide comprising a sequence encoding a guide RNA (gRNA) having a nucleic acid sequence set forth in SEQ ID NO: 19 or SEQ ID NO: 20; and (b) a polynucleotide comprising a sequence encoding a Cas enzyme.

Embodiment 108: A method for producing an edited genome in a plant cell of genus Vitis, the method comprising introducing into the cell a Clustered Regularly Interspaced Short Palindromic Repeats (CRISPR)-associated (CRISPR-Cas) system comprising: (a) at least two guide RNAs (gRNA), or at least one polynucleotide encoding the at least two gRNAs, and (b) an effector protein, or one or more polynucleotides encoding the effector protein; wherein each of the at least two gRNAs hybridize to a Gret1 sequence, and each of the at least two gRNAs form a complex with the effector protein; and wherein the effector protein is a Cas protein comprising a nuclease and/or an effector domain.

Embodiment 109: The method of embodiment 108, wherein the Cas protein is a Cas9 protein, a Cpf1 protein, or a Csm1 protein.

Embodiment 110: The method of embodiment 108 or 109, wherein the edited genome results in a change in the color of a plantlet produced by a plant derived from the plant cell from green to red.

Embodiment 111: The method of embodiment 108 or 109, wherein the edited genome results in a change in the color of a fruit produced by a plant derived from the plant cell from green to red.

Embodiment 112: The method of embodiment 108 or 109, wherein a plantlet produced by a plant derived from the plant cell comprising the edited genome is red, and wherein a plantlet produced by a plant derived from a plant cell that does not comprise the edited genome is green.

Embodiment 113: The method of embodiment 108 or 109, wherein a fruit produced by a plant derived from the plant cell comprising the edited genome is red, and wherein a fruit produced by a plant derived from a plant cell that does not comprise the edited genome is green.

Embodiment 114: A method for producing an edited genome in a plant cell of genus Vitis, the method comprising introducing into the cell a Clustered Regularly Interspaced Short Palindromic Repeats (CRISPR)-associated (CRISPR-Cas) system comprising: (a) at least one guide RNA (gRNA), or at least one polynucleotide encoding the at least one gRNA, and (b) an effector protein, or one or more polynucleotides encoding the effector protein; wherein the at least one gRNA hybridizes to a Gret1 sequence, and the at least one gRNA forms a complex with the effector protein; and wherein the effector protein is a Cas protein comprising a nuclease and/or an effector domain.

Embodiment 115: The method of embodiment 114, wherein the Cas protein is a Cas9 protein, a Cas12a protein, a Cpf1 protein, or a Csm1 protein.

Embodiment 116: The method of embodiment 115, wherein the Cas protein is a Cas12a protein.

Embodiment 117: The method of any one of embodiments 114-116, wherein the edited genome results in a change in the color of a plantlet produced by a plant derived from the plant cell from green to red.

Embodiment 118: The method of any one of embodiments 114-116, wherein the edited genome results in a change in the color of a fruit produced by a plant derived from the plant cell from green to red.

Embodiment 119: The method of any one of embodiments 114-116, wherein a plantlet produced by a plant derived from the plant cell comprising the edited genome is red, and wherein a plantlet produced by a plant derived from a plant cell that does not comprise the edited genome is green.

Embodiment 120: The method of any one of embodiments 114-116, wherein a fruit produced by a plant derived from the plant cell comprising the edited genome is red, and wherein a fruit produced by a plant derived from a plant cell that does not comprise the edited genome is green.

Embodiment 121: The method of any one of embodiments 114-120, wherein the at least one gRNA comprises a nucleotide sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

Embodiment 122: The method of any one of embodiments 114-121, wherein the at least one gRNA comprises SEQ ID NO: 26.

Embodiment 123: The method of any one of embodiments 114-121, wherein the at least one gRNA comprises SEQ ID NO: 28.

Embodiment 124: The method of any one of embodiments 114-121, wherein the at least one gRNA comprises SEQ ID NO: 32.

Embodiment 125: The method of any one of embodiments 114-121, wherein the system comprises at least two gRNAs.

Embodiment 126: The method of embodiment 125, wherein the at least two gRNAs comprise SEQ ID NO: 26 and SEQ ID NO: 33.

Embodiment 127: The method of embodiment 125, wherein the at least two gRNAs comprise SEQ ID NO: 34 and SEQ ID NO: 35.

Embodiment 128: The method of any one of embodiments 108-127, wherein the plant cell is a SUGRA35 cell.

Embodiment 129: The method of any one of embodiments 108-128, wherein the Cas protein is optimized for expression in the Vitis cell.

Embodiment 130: A genetically engineered Vitis cell produced by the method of any one of embodiments 108-129.

Embodiment 131: A genetically engineered Vitis plant comprising the cells produced by the method of any one of embodiments 108-129.

Embodiment 132: A product comprising the genetically engineered plant of embodiment 131, or portions thereof, wherein the product has altered pigmentation relative to a product produced by a non-genetically engineered plant of the same strain.

Embodiment 133: The product of embodiment 132, wherein the product comprises a fruit.

Embodiment 134: The product of embodiment 132 or 133, wherein the product comprises a grape.

Embodiment 135: The product of embodiment 132 or 133, wherein the product is a wine.

Embodiment 136: A guide RNA (gRNA) comprising the nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

Embodiment 137: A composition comprising: (a) a polynucleotide comprising a sequence encoding a guide RNA (gRNA) having a nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35; and (b) a polynucleotide comprising a sequence encoding a Cas enzyme.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: General Materials and Methods

### Plant Material and Tissue Cultures

Shoot apical meristems were collected from greenhouse grown plants. Embryogenic cultures were induced using leaf explants following a protocol that was previously described in Dhekney et al., In Vitro Embryogenesis in Higher Plants (pp. 263-277) (2016). Thompson seedless (TS) were used for PDS1 gene editing. Anthocyanin-over-expressed Thompson seedless (AOTS) and Merlot were used for mybA1 gene editing.

### Example 2: pHEE-PDS1 and pHDE-PDS1 CRISPR-Cas9 Vector Construction

A binary vector containing two gRNAs each targeting different portions of the PDS1 gene were used to generate a large deletion in a PDS1 gene in a plant cell. The deletion was engineered to occur between the sequences targeted by the two gRNAs resulting in an unambiguous knockout. The deletion was performed in a manner similar to that of Gao et al., Plant physiology, 171(3), 1794-1800, (2016), which is incorporated herein by reference in its entirety.

PDS1 gRNA1 &2 construction

The PDS1 gene sequences of Vitis vinifera Pinot Noir (PN) and Vitis vinifera Thomson seedless (TS) were aligned, and gRNAs were designed to target conserved exon-containing regions. The PDS1 gene sequence of Vitis vinifera Pinot Noir (PN) is provided in GenBank NCBI reference sequence NC_012015.3, c95502-70435, Vitis vinifera cultivar PN40024 chromosome 9 (available at ncbi.nlm.nih.gov/nuccore/NC_012015.3?report=fasta&from=70435&to=95502&strand=true), also shown in SEQ ID NO: 1.

The sequences of PDS1 gRNA 1 and PDS1 gRNA 2, used to target the PDS1 gene, are shown below in Table 2. PDS1 gRNA 1 targets positions 92362-92342 of GenBank NCBI reference sequence NC_012015.3, and PDS1 gRNA2 targets positions 81704-81685 of GenBank NCBI reference sequence NC_012015.3.

TABLE 2
PDS1 gRNA sequences

| PDS1 gRNA | Nucleotide Sequence | SEQ ID NO |
|---|---|---|
| PDS1 gRNA 1 | AGGTTGTAATTGCTGGTGCAGG | 2 |
| PDS1 gRNA 2 | CTTATGTAATTGCAACTCCAGG | 3 |

pHEE-PDS1 Vectors Construction and Validation

Plasmid vectors containing PDS1-gRNA1 and PDS1-gRNA2 were generated. A “gRNA1&2 related piece” that contains PDS1-gRNA1 and PDS1-gRNA2 was generated from a pHEE-AT5G plasmid vector comprising a “gRNA1&2 related piece.” The gRNA1&2 related piece of the pHEE-AT5G plasmid vector was PCR amplified, and a “gRNA1&2 related piece” containing PDS1-gRNA1 and PDS1-gRNA2 (a “PDS1 gRNA1&2 related piece”) was generated as follows:

Primers used for the amplification of the PDS1-gRNA1&2 piece were PDS1-crp1 and PDS1-crp2, and primers for the amplification of the mybA1I-gRNA 1&2 piece were mybA1-crp1 and mybA1-crp2. The primer sequences are set forth in Table 3, below.

TABLE 3
Primer Sequences

| Primer | | SEQ |
|---|---|---|
| Name | Nucleotide Sequence | ID NO |
| PDS1-crp1 | CTAGAGTCGAAGTAGTGATTGAGGTTGTAATT | 4 |
| | GCTGGTGCGTTTTAGAGCTAGAAATAGC | |
| PDS1-crp2 | TGCTATTTCTAGCTCTAAAACGGAGTTGCAAT | 5 |
| | TACATAAGCAATCTCTTAGTCGACTCTA | |
| mybA1-crp1 | CTAGAGTCGAAGTAGTGATTGGATGCTTATCG | 6 |
| | CAAGCCTCGTTTTAGAGCTAGAAATAGC | |
| mybA1-crp2 | TGCTATTTCTAGCTCTAAAACCTCCGAGCAGG | 7 |
| | TAACATGACAATCTCTTAGTCGACTCTA | |
| U626-IDF | TGTCCCAGGATTAGAATTAGGC | 8 |
| U629-IDR | AGCCCTCTTCTTTCGATCCATCAAC | 9 |
| pHDE-Pmel | CCTGTCAAACACTGATAGTTTCGACTTGCCTT | 10 |
| 5p | CCGCACAATACATC | |
| pHDE-Pmel | CAAGCTTCACTTCACTAGTTTGCTTTTATTCA | 11 |
| 3p | CTAGTATAACCATGGTATTGG | |
| PDS1-GT1 | TTGGAGGTGGTGACTCTATGGGTC | 12 |
| PDS1-GT2 | CACCCAAACCCCTCCAACAAACC | 13 |
| PDS1-GT3 | CCCTAATTCATCACTCCATACCAGC | 14 |
| mybA1-GT1 | ATGGAGAGCTTAGGAGTTAGAAAGGG | 15 |
| mybA1-GT2 | TCAGATCAAGTGATTTACTTGTGTGTTGGG | 16 |

PCR reactions were performed in a total volume of 100 μl comprising 1 ul pHTEE-AT5G plasmid, 20 μl of 5*Phusion buffer, 2 mM DNTP, 1 μl of each primer, and 2.5 μl of Phusion DNA polymerase (NEB, M0530). The PCR cycling conditions used for amplification were an initial denaturation at 98° C. for 3 min; 10 cycles of 98° C. for 10 s, 58° C. for 20 s, 72° C. for 20 s; and 35 cycles of 98° C. for 10 s, 72° C. for 20 s. The PCR products were initially visualized by electrophoresis using a 1% agarose gel.

PCR product was extracted from gel by freezing the gel in −20° C. around 15 min, then centrifuged at 15000 rpm for 5 min. The final product was used in the Gibson assembly as a “gRNA1&2 related piece.”

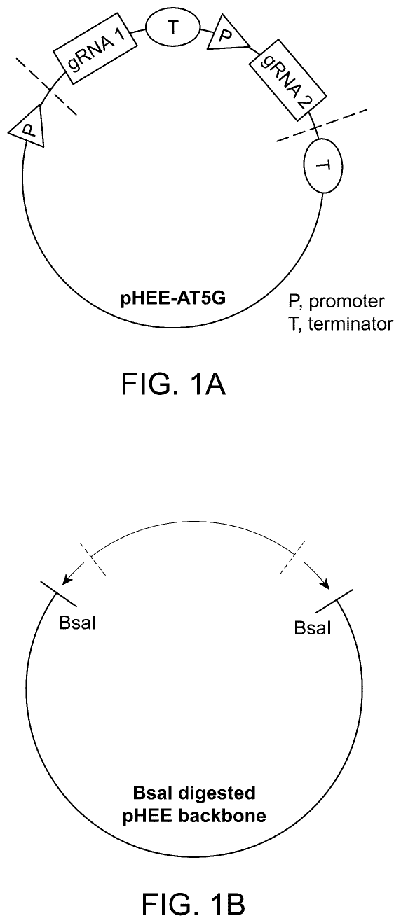

The PDS1-gRNA1 and PDS1-gRNA2 sequences were introduced using primer sequences engineered to include said gRNA sequences. In addition to PDS1-gRNA1 and PDS1-gRNA2 sequences, the resulting PDS1 gRNA1&2 related piece contained: gRNA1, terminator of gRNA1, promoter of gRNA2, and gRNA2. The PDS1 gRNA1&2 related piece was also engineered to facilitate Gibson Assembly. A sequence of a gRNA1&2 related piece, with additional flanking sequences of the promoter of gRNA1 on the 5′ side, and the terminator of gRNA2 on the 3′ side, is set forth as SEQ ID NO: 17. PDS1-gRNA1 was engineered to be positioned in the first (more 5′) region denoted by a series of “N.” PDS1-gRNA2 was engineered to be positioned in the second (more 3′) region denoted by a series of “N.” Referring to FIG. 1A, following assembly into the vector, gRNA1 was positioned in a region flanked by a U6-26 promoter sequence on the 5′ side, and a U6-26 terminator sequence on the 3′ side. Also referring to FIG. 1A, gRNA2 was positioned in a region flanked by a U6-29 promoter sequence on the 5′ side, and a U6-29 terminator sequence on the 3′ side.

(SEQ ID NO: 17)
AAGCTTCGACTTGCCTTCCGCACAATACATCATTTCTTCTTAGCTTTTTT
TCTTCTTCTTCGTTCATACAGTTTTTTTTTGTTTATCAGCTTACATTTTC
TTGAACCGTAGCTTTCGTTTTCTTCTTTTTAACTTTCCATTCGGAGTTTT
TGTATCTTGTTTCATAGTTTGTCCCAGGATTAGAATGATTAGGCATCGAA
CCTTCAAGAATTTGATTGAATAAAACATCTTCATTCTTAAGATATGAAGA
TAATCTTCAAAAGGCCCCTGGGAATCTGAAAGAAGAGAAGCAGGCCCATT
TATATGGGAAAGAACAATAGTATTTCTTATATAGGCCCATTTAAGTTGAA
AACAATCTTCAAAAGTCCCACATCGCTTAGATAAGAAAACGAAGCTGAGT
TTATATACAGCTAGAGTCGAAGTAGTGATTGNNNNNNNNNNNNNNNNNNN
GTTTTAGAGCTAGAAATAGCAAGTTAAAATAAGGCTAGTCCGTTATCAAC
TTGAAAAAGTGGCACCGAGTCGGTGCTTTTTTTTGCAAAATTTTCCAGAT
CGATTTCTTCTTCCTCTGTTCTTCGGCGTTCAATTTCTGGGGTTTTCTCT
TCGTTTTCTGTAACTGAAACCTAAAATTTGACCTAAAAAAAATCTCAAAT
AATATGATTCAGTGGTTTTGTACTTTTCAGTTAGTTGAGTTTTGCAGTTC
CGATGAGATAAACCAATATTAATCCAAACTACTGCAGCCTGACAGACAAA
TGAGGATGCAAACAATTTTAAAGTTTATCTAACGCTAGCTGTTTTGTTTC
TTCTCTCTGGTGCACCAACGACGGCGTTTTCTCAATCATAAAGAGGCTTG
TTTTACTTAAGGCCAATAATGTTGATGGATCGAAAGAAGAGGGCTTTTAA
TAAACGAGCCCGTTTAAGCTGTAAACGATGTCAAAAACATCCCACATCGT
TCAGTTGAAAATAGAAGCTCTGTTTATATATTGGTAGAGTCGACTAAGAG
ATTGNNNNNNNNNNNNNNNNNNNGTTTTAGAGCTAGAAATAGCAAGTTAA
AATAAGGCTAGTCCGTTATCAACTTGAAAAAGTGGCACCGAGTCGGTGCT
TTTTTTTGCAAAATTTTCCAGATCGATTTCTTCTTCCTCTGTTCTTCGGC
GTTCAATTTCTGGGGTTTTCTCTTCGTTTTCTGTAACTGAAACCTAAAAT
TTGACCTAAAAAAAATCTCAAATAATATGATTCAGTGGTTTTGTACTTTT
CAGTTAGTTGAGTTTTGCAGTTCCGATGAGATAAACCAATA

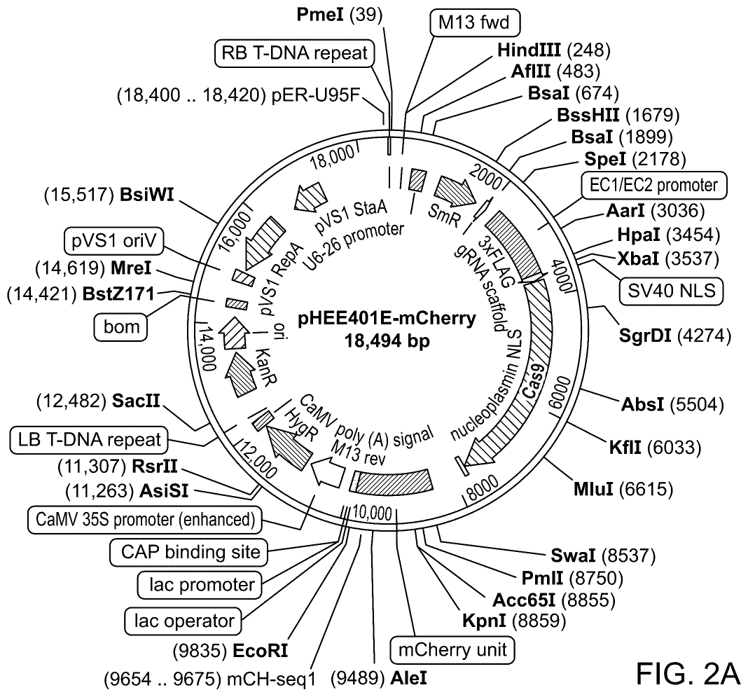

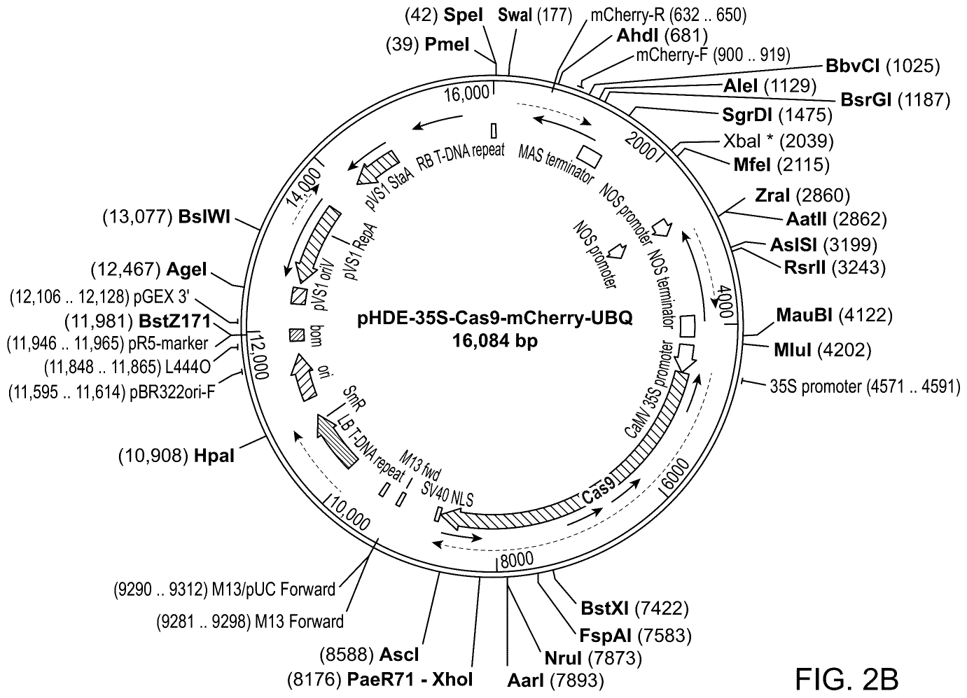

The PDS1 gRNA1&2 related piece was cloned into BsaI digested pHEE vector by Gibson assembly (FIGS. 1A-1B). 4 μl ligation product was mixed with 50 ul E. coli competent cells (Invitrogen, 18258012) and placed on ice for 20 min. 42° C. heat shock was applied for 90 s. And the mixture was placed on ice for 3 min, then incubated in 37° C. for 30 min. The product was spread on solid LB (Lysogeny broth) media (kanamycin 100 ul/100 ml). Cells were incubated in 37° C. overnight. Colony PCR reactions were performed in a total volume of 10 μl comprising 5 μl of Platinum II Hot-Start PCR Master Mix (Invitrogen, 14000-012), 0.5 μl of amplification primer, and E. coli colony. The PCR cycling conditions used for amplification were an initial denaturation at 94° C. for 2 min; 40 cycles of 94° C. for 30 s, 58° C. for 30 s, 72° C. for 45 s. Plasmids were extracted from positive colony's culture using Plasmid Plus Midi Kit (Qiagen, 12943) and sequenced for confirmation. Description of the pHEE backbones (pHEE401E vector) is provided in FIG. 2A. Cas9 was driven by a EC1/EC2 promoter.

pHDE-PDS1 vectors construction and validation

A PDS1 gRNA1&2 related piece, plus the 5′ promoter of gRNA1 and 3′ terminator of gRNA2, was cloned from the pHEE-PDS1 vector using PCR amplification. The new PDS1 gRNA1&2 related piece for pHDE vector was cloned into a PmeI digested pHDE vector by Gibson assembly. Detailed descriptions of the pHDE backbones (pHDE-35S-Cas9-mCherry-UBQ) are provided in FIG. 2B. Cas9 was driven by a CaMV 35S promoter. The vector was constructed and validated as follows.

Primers used for the amplification of PDS1-gRNA1&2 piece, plus the promoter of gRNA1 and terminator of gRNA2, were pHDE-Pmel 5p and were pHDE-Pmel 3p (See Table 3). PCR reactions were performed in a total volume of 100 μl comprising 1 ul pHEE-PDS1 plasmid, 20 μl of 5*Phusion buffer, 2 mM DNTP, 1 μl of each primer, and 2.5 μl of Phusion DNA polymerase (NEB, M0530). The PCR cycling conditions used for amplification were an initial denaturation at 95° C. for 2 min; 40 cycles of 95° C. for 30 s, 60° C. for 30 s, 72° C. for 30 s. The PCR products were initially visualized by electrophoresis using a 1% agarose gel. PCR product was extracted from gel by freezing the gel in −20° C. around 15 min, then centrifuged at 15000 rpm for 5 min.

### Example 3. Transformation and Detection Methods

### Plant Transformation and Regeneration

The pHEE-PDS1 plasmids were transformed into Agrobacterium strain GV3101 via electroporation. Agrobacterium-mediated plant transformation and plant regeneration were performed as described in Dhekney et al. 2016, which is incorporated herein by reference in its entirety.

### Detection of Gene Edits

Genomic DNA was extracted from calli using DNeasy Plant Pro Kit (Qiagen, 69206) following the supplier's instructions. The success of the deletion was verified by PCR. Detection was carried out as follows.

Primers PDS1-GT1, PDS1-GT2, and PDS1-GT3 (Table 3) were used to determine whether the expected deletion is generated by CRISPR-Cas9 in the PDS1 gene. Sequencing confirmation of large deletion between gRNA1 and gRNA2 was performed at the calli stage.

PCR reactions were performed in a total volume of 10 μl comprising 5 μl of Platinum II Hot-Start PCR Master Mix (Invitrogen, 14000-012), 0.5 μl of forward and reverse primers, and 1 μl of plant DNA. The PCR cycling conditions used for amplification were an initial denaturation at 94° C. for 2 min; 45 cycles of 94° C. for 30 s, 60° C. for 30 s, 72° C. for 50 s.

PDS1 edited TS plants were visually recorded for phenotyping at the calli stage.

### Example 4: Generation of PDS1 Gene Edits in Thompson Seedless by CRISPR-Cas9

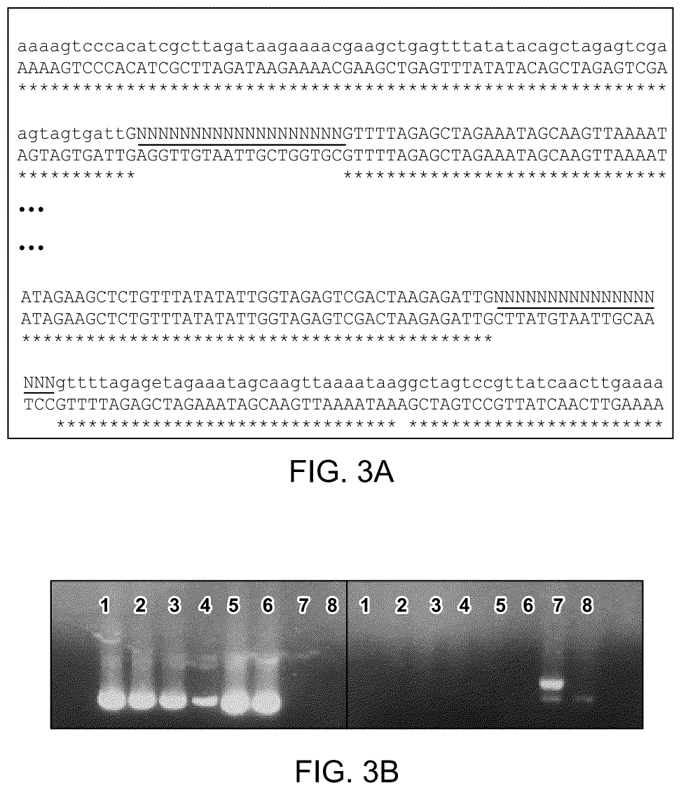

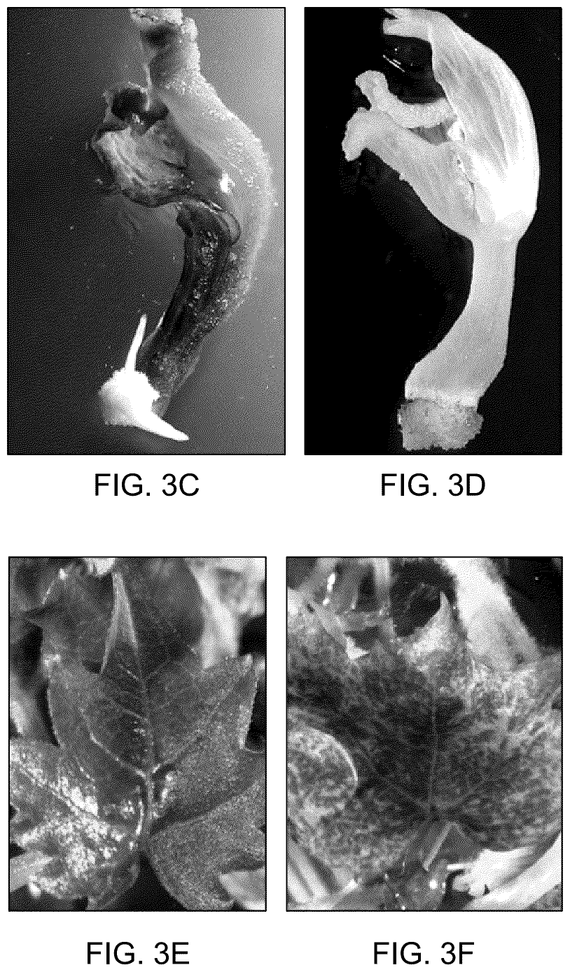

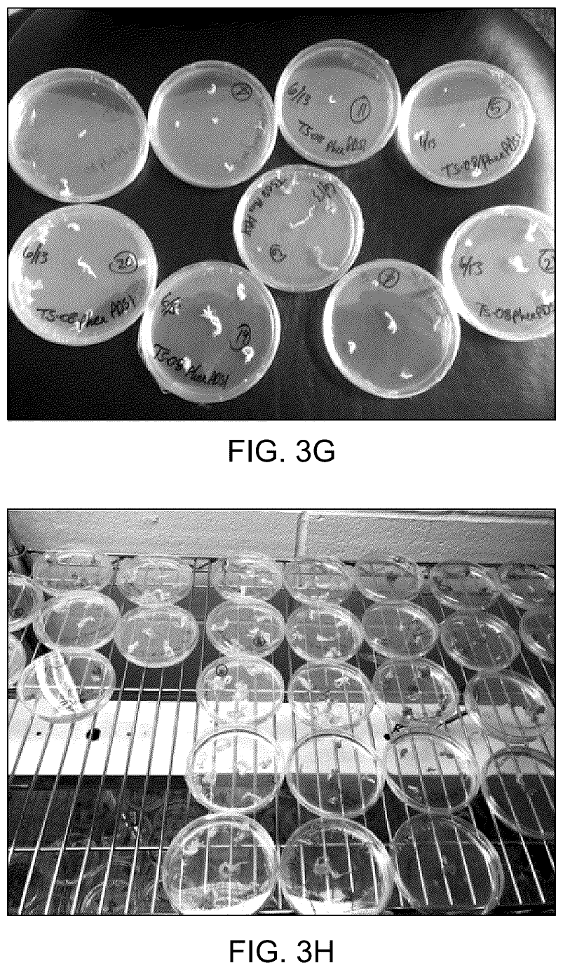

Proper PDS1 gRNA1&2 related piece generation and insertion into the vector was confirmed by sequencing (FIG. 3A). pHEE-PDS1 and pHDE-PDS1 vectors were transformed into wild-type Vitis vinifera Thompson Seedless cells through Agrobacteria transformation, separately, in order to generate large deletions in the PDS1 gene as described above. Eight of pHEE-PDS1 treated callus clusters were randomly selected for DNA extraction, and two of them were confirmed to contain the large deletion through PCR (FIG. 3B). There were no PCR bands detected among eight randomly selected pHDE-PDS1 treated callus clusters (while some of them still show edited phenotype in plantlet stage).

After calli developed into plantlets, PDS1 edited plantlets showed albino (FIG. 3D) or green-white mosaic phenotype (FIG. 3F), whereas non-edited wildtype TS plantlets had a green color (FIGS. 3C and 3E). 16.7% of pHEE-PDS1 transformed plantlets (7 of full phenotype and 3 of mosaic phenotype plantlets out of 60 total lines) and 33.3% of pHDE-PDS1 transformed plantlets (13 full phenotype and 0 mosaic out of 39 total lines) exhibited mutated phenotypes (FIGS. 3G and 3H).

Accordingly, these results demonstrate that the methods of the present technology are useful for producing edited genomes in Vitis plant cells and are effective for producing a change in the color of a plantlet produced by a plant derived from the Vitis plant cells.

### Example 5: Generation of pHEE-mybA1 and pHDE-mybA1 CRISPR-Cas9 Vector Containing gRNA1 and gRNA2

The skin color of grapes is determined by the quantity and composition of anthocyanins. Black and red cultivars accumulate anthocyanin in their skins, but white cultivars do not synthesize anthocyanins (Azuma et al. 2007). The key enzyme responsible for the accumulation of anthocyanins in grape berry skins is UDP-glucose:flavonoid 3-o-glucosyltransferase (UFGT), and its expression is transcriptionally regulated by MybA transcription factors. The MybA genes are closely clustered in a single locus, referred to as the berry color locus. It has been reported that the insertion of the retrotransposon Gret1 into the promoter region of the MybA1 or nucleotide mutations in the MybA2 gene can result in the white berry allele of the MybA2 gene (Ford et al. 1998, Kobayashi et al. 2004, Walker et al. 2007).

The mybA1 gene sequence from Vitis Vinifera ‘Pinot Noir’ (PN) is provided in GenBank NCBI reference sequence CP126649.1, location 16271055-16270097, Vitis vinifera cultivar Pinot Noir 40024 chromosome 2 (available at ncbi.nlm.nih.gov/nucleotide/CP126649.1?report=genbank&log$=nuclalign&blast_rank=1&R ID=EFMRV81Y016&from=16270097&to=16271055), also shown below in SEQ ID NO: 18. Exonic regions are shown in underline, and exemplary sequences targeted by gRNAs are shown in bold text. The sequence as shown in SEQ ID NO: 18 was used design gRNAs for use in the experiments described below to target mybA1 of Vitis Vinifera anthocyanin over-expressed Thompson Seedless (AOTS) and Vitis Vinifera ‘Merlot’, since there is significant sequence conservation between the mybA1 genes of PN, AOTS, and Merlot.

(SEQ ID NO: 18)
ATGGAGAGCTTAGGAGTTAGAAAGGGTGCATGGATCCAAGAAGAGGATGT
TCTCCTGAGGAAATGCATTGAGAAATATGGAGAAGGAAAGTGGCATCTGG
TTCCCCTCCGAGCAGGTAACATGAAAGAGAAAGGGATCAGTATTTATTTG
TGTTTTTTTACTTCTGTTTTGCTTAAAGAGTTTCATTTTCTTGAGTTTGC
AGGGTTGAATAGATGCCGAAAAAGCTGCAGGTTGAGATGGCTCAATTATT
TGAAGCCGGATATCAAGAGAGGAGAGTTTGCATTAGACGAGGTTGATCTC
ATGATTAGGCTTCACAATTTGTTGGGGAACAGGCAAGTCTATAATAACTC
AAGTACTAGCTTGATAATGATATTATATTAGTTCTGAAGCTGTTCAGAAC
TTACAAAAGAGCTGTTCAGTTGATACTTTGTCTGATGTTGTGCGTGTATA
GATGGTCCTTGATTGCGGGTAGGCTTCCAGGGAGGACTGCTAATGATGTC
AAGAACTATTGGCATAGTCACCACTTCAAAAAGGAGGTTCAGTTCCAGGA
AGAAGGGAGAGATAAACCCCAAACACATTCTAAAACCAAAGCTATAAAGC
CTCACCCTCACAAGTTCTCCAAAGCCTTGCCAAGGTTTGAACTAAAAACT
ACAGCTGTGGATACTTTTGACACACAAGTCAGTACTTCCAGGAAGCCATC
ATCCACTTCACCACAACCGAATGATGACATCATATGGTGGGAAAGCCTGT
TAGCTGAGCATGCTCAAATGGATCAAGAAACTGACTTTTCGGCTTCTGGA
GAGATGCTTATCGCAAGCCTCAGGACAGAAGAAACTGCAACACAGAAAAA
GGGACCCATGGATGGTATGATTGAACAAATCCAGGGAGGTGAGGGTGATT
TTCCATTTGATGTGGGCTTCTGGGATACACCCAACACACAAGTAAATCAC
TTGATCTGA.

A mybA1 gene sequence from Vitis Vinifera ‘Merlot’ is provided in GenBank NCBI reference sequence GU145120.1, Vitis vinifera cultivar Merlot MybA1 (mybA1) gene, mybA1-SUB allele, partial cds (available at ncbi.nlm.nih.gov/nuccore/GU145120.1).

Two gRNAs targeting the MybA1 gene (mybA1 gRNA1 and mybA1 gRNA2) were designed to be installed into a gRNA1&2 related piece (a “mybA1 gRNA1&2 related piece”), and the mybA1 gRNA1&2 related piece was inserted into pHEE backbone through BsaI site in a manner similar to that described above for the generation of pHEE-PDS1. The mybA1 gRNA1&2 related piece plus the promoter of gRNA1 and terminator of gRNA2 were inserted into PmeI digested pHDE backbone in a manner similar to that described above for the generation of pHDE-PDS1. In the vector, mybA1 gRNA1 was flanked by a u6-26 promoter and a u6-26 terminator, and mybA1 gRNA2 was flanked by a u6-29 promoter and a u6-29 terminator. Successful generation of the vector was confirmed by sequencing (data not shown). The sequences of mybA1 gRNA 1 and mybA1 gRNA 2 are shown below in Table 4. mybA1 gRNA 1 targets positions 16270951-16270932 of GenBank NCBI reference sequence CP126649.1, and mybA1 gRNA2 targets positions 16270254-16270235 of GenBank NCBI reference sequence CP126649.1.

TABLE 4
mybA1 gRNA sequences

| PDS1 gRNA | Nucleotide Sequence | SEQ ID NO |
|---|---|---|
| mybA1 gRNA 1 | GATGCTTATCGCAAGCCTCAGG | 19 |
| mybA1 gRNA 2 | TCATGTTACCTGCTCGGAGGGG | 20 |

### Example 6: Generation of mybA1 Edits in Anthocyanin Over-Expressed Thompson Seedless (AOTS) by CRISPR-Cas9

pHEE-MybA1 and pHDE-MybA1 vectors were transformed into AOTS cells through Agrobacteria transformation, separately, in order to introduce large inactivating deletions within the mybA1 gene. Sixteen of pHEE-PDS1 treated callus clusters were randomly selected for DNA extraction and two of pHEE-PDS1 treated callus clusters were confirmed by gel electrophoresis. MybA1 edits were detected using mybA1-GT1 and mybA1-GT2 primers (Table 3). The deletion was also confirmed by sequencing. Sequencing confirmation of large deletion between gRNA1 and gRNA2 was performed at the calli stage, using the primer mybA1-GT1 (Table 3) for mybA1 mutants.

MybA1 gene edited AOTS were visually recorded for phenotyping at the calli stage.

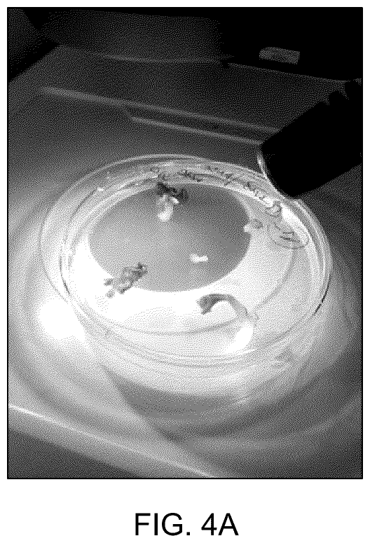

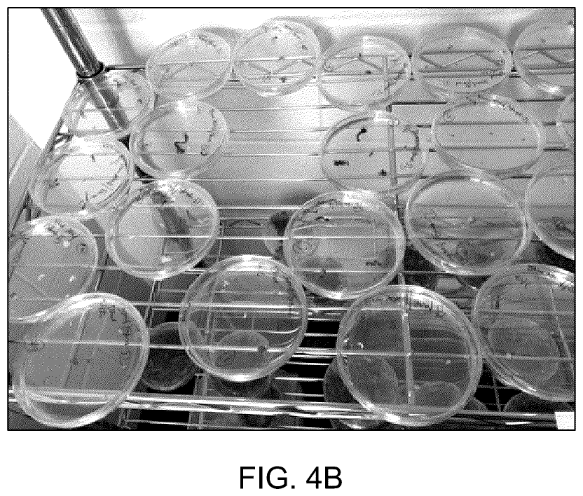

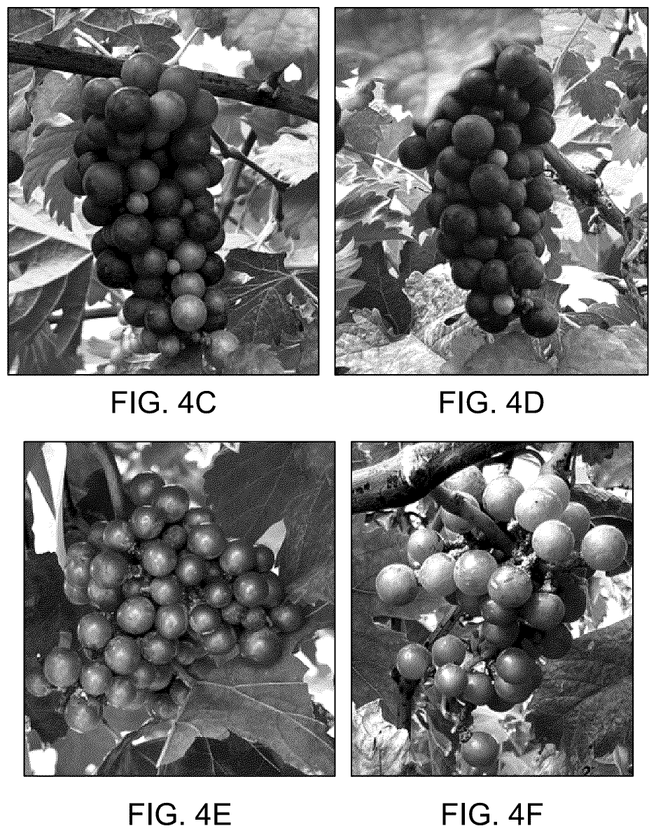

Wild-type AOTS plantlets exhibited a red color, while MybA1-edited AOTS plantlets exhibited a green color (FIGS. 4A and 4B). 66.7% of pHDE-MybA1 transformed plantlets (18 out of 27 total lines) exhibited mutated phenotypes (FIG. 4B).

Accordingly, these results demonstrate that the methods of the present technology are useful for producing an edited genome in Vitis plant cells and are effective for producing a change in the color of a plantlet produced by a plant derived from the Vitis plant cells.

### Example 7: Generation of mybA1 Edits in Merlot by CRISPR-Cas9

pHEE-MybA1 and pHDE-MybA1 vectors were transformed into Vitis vinifera Merlot cells through Agrobacteria transformation, separately, in order to introduce large inactivating deletions within the mybA1 gene. The large deletion between gRNA1 and gRNA2 of MybA1 was confirmed by sequencing (data not shown). MybA1 edited Merlot plants were visually recorded for phenotyping at fruiting stage.

At the plantlet stage, both wild-type of Merlot plantlets and MybA1-edited Merlot plantlets were green color. The phenotype of MybA1-edited Merlot plants were evaluated at the fruiting stage. The fruits of wild-type Merlot developed from green, to mixed green and purple (early fruiting stage), to dark purple (mature (late) fruiting stage) (FIGS. 4C-D), while the fruits of MybA1-edited Merlot plants maintained a green color from the early fruiting stage through mature fruiting stage (FIGS. 4E-F).

Accordingly, these results demonstrate that the methods of the present technology are useful for producing an edited genome in Vitis plant cells and are effective for producing a change in the color of a plantlet produced by a plant derived from the Vitis plant cells.

### Example 8: Generation of Gret1 Edits in Plants Producing White Grapes to Induce Plantlet and Berry Color Change

### Gret1 Vector Construction

A binary vector containing two gRNAs, each targeting different portions of a Gret1 sequence, is used to generate a large deletion in or complete deletion of the Gret1 sequence in a plant cell, preferably a SUGRA35 cell. The deletion is engineered to occur between the sequences targeted by the two gRNAs, resulting in an unambiguous knockout. The Gret1 vector is analogous to the pHEE-PDS1 vector described above. Two gRNAs are installed in a Gret1-gRNA1&2 related piece. Restriction enzyme digested vector backbone is ligated with the Gret1-gRNA1&2 related piece, and the ligation product (Gret1 vector) is generated. The Gret1 vector may also encode a Cpf1 (Cas12a) enzyme. The Gret1 vector is transformed into E. coli competent cells. Colony PCR and sequencing are performed for Gret1 vector validation.

### Plant Transformation and Regeneration

The Gret1 vector (plasmids) are transformed into bacteria via electroporation. Bacterium-mediated plant transformation and plant regeneration are performed as previously described (Dhekney et al. 2016).

### Detection of Edits at Callus, Plantlet and Fruit Stage

Genomic DNA is extracted from calli using a DNeasy Plant Pro Kit (Qiagen, 69206) according to the manufacturer's protocol. PCR validation is performed using screening primers. Edited plants are visually recorded for phenotype at the fruit stage. qPCR is conducted to check the Gret1 expression level. The editing events are confirmed by sequencing.

Accordingly, it is anticipated that these results will demonstrate that the methods of the present technology are useful for producing an edited genome in Vitis plant cells and are effective for producing a change in the color of a plantlet produced by a plant derived from the Vitis plant cells.

### Example 9: Gret1-Based Grape Editing Systems and Development Thereof

### Gret1-Based Gene Editing in Grape Protoplasts

Applicant designed guide RNA sequences targeting in Gret1. Guides were identified by analyzing putative protospacer adjacent motif (PAM) sites, filtering for an appropriate GC-content, removing candidates with repetitive sequences, and checking off-target activity. Guides were selected to span the whole Terminal Inverted Repeat (TIR) and have minimal overlap between guides. This enables the targeting of TIRs flanking the Gret1 gene with a single gRNA for the purposes of generating a large deletion. In addition, other guide RNAs were identified that can be used in a pairwise fashion, such that two gRNAs are used to accomplish a large deletion in the Gret1 gene. A list of the 10 identified gRNAs are shown below in Table 5.

TABLE 5
Gret1-targeting gRNAs

| gRNA # | Sequence (5′−>3′) | PAM | GC% | Placement | SEQ ID NO. |
|---|---|---|---|---|---|
| 1 | CTCTTCCTTGGACAGAAGAAGCT | TTTG | 47.8 | Identical | 26 |
| | | | | in both | |
| | | | | TIRs | |
| 2 | ATCGTCCGGATCATCAATTAAAG | TTTA | 39.1 | Identical | 27 |
| | | | | in both | |
| | | | | TIRs | |
| 3 | CCACGATATCTAGACAGCAGCAT | TTTC | 47.8 | Identical | 28 |
| | | | | in both | |
| | | | | TIRs | |
| 4 | TCATGATGACATTCGGAGGCAGC | TTTG | 52.2 | Identical | 29 |
| | | | | in both | |
| | | | | TIRs | |
| 5 | CCAGGTATTAAGAAGTTACCAGG | TTTA | 43.5 | Identical | 30 |
| | | | | in both | |
| | | | | TIRs | |
| 6 | ATCCAGTCGTCATTCCTGCAAGA | TTTG | 47.8 | Identical | 31 |
| | | | | in both | |
| | | | | TIRs | |
| 7 | TGAGTTGAGATCACGCGTCCATC | TTTA | 52.2 | Identical | 32 |
| | | | | in both | |
| | | | | TIRs | |
| 8 | GCGGTCCACGTAGTAACCAGATG | TTTG | 56.5 | Identical | 33 |
| | | | | in both | |
| | | | | TIRs | |
| 9 | CCAGAGGGAGGGGTCCCTACATG | TTTC | 65.22 | Unique to | 34 |
| | | | | 5′ ITR | |
| 10 | TCCATGAACTCCAGCGCATTTGG | TTTG | 52.17 | Unique to | 35 |
| | | | | 3′ TIR | |
| | | | | (start of 5′ UTR) | |

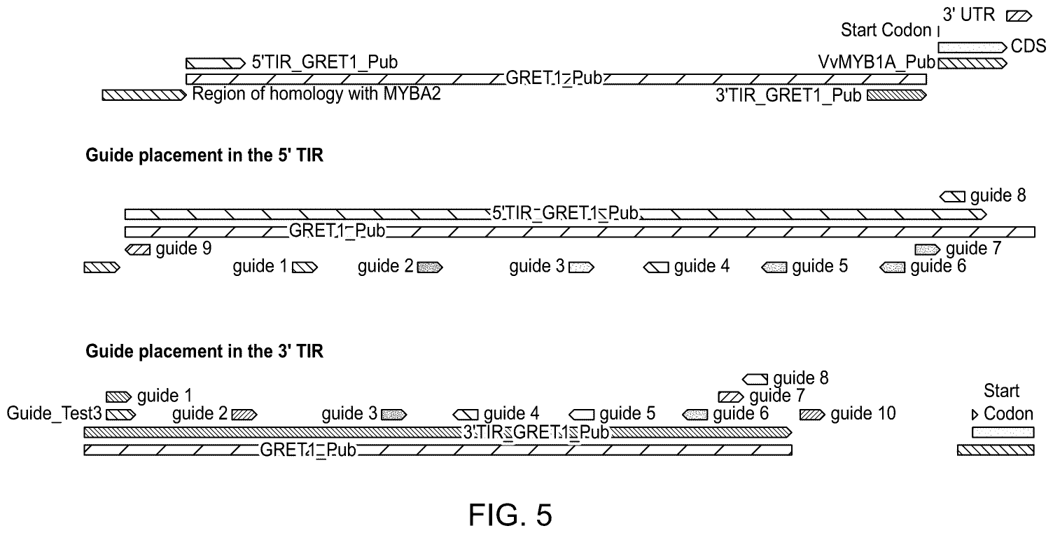

A diagram of the placement of the various gRNAs relative to the structure of Gret1 and MYBA1 is shown in FIG. 5.

The expected repair outcomes are summarized below in Table 6. To arrive at these sequences, the intervening sequences between the locations of gRNA targeting were removed, for each gRNA or combination of gRNAs tested.

TABLE 6
Expected post-edit Gret1 sequences

| Indicator | Description | Sequence | SEQ ID NO: |
|---|---|---|---|
| SW7_GR | SW7 | TAAATTAAAACATGAGATTTTTGAAGGATTAG | 36 |
| ET_MYB | MYBA1 loci | ACTAGATAGATAGTTGAATATAGGATAATAAT | |
| A1 | | TTTAATTGATGAACATAAAATTTAGACGAATA | |
| | | TTTGAAGAAGTAAATTAAGACATGTGATTTTTT | |
| | | TGATGGATTAGACTTTGAATAACTGATAAATA | |
| | | TGAGACAAGGATTCGATTTATGAAAATAAGAT | |
| | | TTAAACAAATATTTGAAGAATTAAATTAAAAC | |
| | | AAGGGATTTTTGAAGGATTGACATAAGACCCT | |
| | | TCATCCACACGAACTAGGCCTGGACGCCAACT | |
| | | CAAGCCCAAGTCCAATGTCTTCAGGGGCAAAC | |
| | | CCAATACAACCATCCAACATAGGTAAGCCCAA | |
| | | AACCCAATGGCTTCAAGGGCAAGCCCAAAACA | |
| | | AGCATCCCCATCACAAGCTTGGGCCTAGTTCCT | |
| | | CAACCCAAGCCCATGTTCAATGCTAATATGGG | |
| | | CGGGCTCAAAGACCCATCTTCCAACAGTATTTT | |
| | | AATTTGAAAAACCCAAAACTACAAGCTTGAAA | |
| | | CTGGAAAACTCATAAGATACCTATCAAAACAT | |
| | | GGAAGTGGAGCCAAAGATGAGGGGGAGGAGG | |
| | | CGGAGCTACGGGAAGGATGAAGGGAGGAGGA | |
| | | GCTCCAATGCCGCCATTGATTCAGCTGACTGA | |
| | | GCGAGCTTGAGGAAATGGGTATGAAAGGTGGC | |
| | | GGCCATGCATGGCTATGCATGGCCATGCGGGG | |
| | | GTGAGGGGAATGGGCGTAATGGGCATGGGTGG | |
| | | GAACTAAGATAGCTGGAACATGGTATGGGTTC | |
| | | AATGTGGCCATGCCCGGCCATGCAAGGGTGGA | |
| | | GAGAACTGGAAGTTCTCAATGCCGTAATCAAA | |
| | | CATCAAACCCAGGTGGGAATCCAACCTCTTGC | |
| | | AGGGGAAAAATCAACAAAATTATAGAAAGCA | |
| | | AAAAAAACAGAGCAAGCACATGTAGGCAATC | |
| | | GTATTTGAGTAAAACAAATTTGTGGTTTTGGAT | |
| | | ACCCACAGAAAATCACAAAATAATCTTCGTAG | |
| | | ACTAACAGAGGACCTTACCTGAATACTCCACA | |
| | | ACGCCACCCTCCTCTCTCTCGTCTGAATATCCC | |
| | | CACCTCCTCTCCCAAACGTCCTTCTCCTTCTGA | |
| | | AAAGATCAATGTTTCAGAAAAACTCTCTCAAA | |
| | | GGCTCCCTCTCTAAGGTGTCTTCCTCTCTGGTT | |
| | | CCTTTTCCTTCCCATCTCCAGCACTCCTGCCCCT | |
| | | CGAGTGTATCCTTCCTGTTTTTTATTTCTTCCTT | |
| | | GGCAAGCCATTACCAACCTTACTATTCCTTCAT | |
| | | TGCTGGCTGATCAACCACCAGCATGGCCCCTG | |
| | | CCATCTCCATGCAGCCGGTTTCCCGGGAGAAA | |
| | | AGGCCCCCCCCCCCCCCCCGGCCCCCCTTTTTT | |
| | | TAAAAAAGGAAAGCCAAGAAAAAAAAAAAAC | |
| | | TCTCTTCCGGGCCCCCCCCCAAAGGGGGCTAC | |
| | | CAATACCCCCCCATTTAAAAATTTAAGTTTAAA | |
| | | AAATGAAATAATTTGAATAACAATTTTTTTTTA | |
| | | TTTAAAAAAAAAAAAAAGGAAAAAATGAAAT | |
| | | TTTTAAAATTTTCTTTAAGCCCAATATATTTCC | |
| | | CACTAAAGTTTAAAAACTCTTCTCCCAGAAAA | |
| | | AATCCTATTGCTGCCCCTATATTAAACCTTTTT | |
| | | TTTTTTTTTTTTTCCCCTCCTTAAACTGCAAAAG | |
| | | GCTTNCTCTACAGTAATTTGGGGCAAGTTATGA | |
| | | GAGTTGTGATTCTGAGCCCTGAGAAAATGGAA | |
| | | ATTTCATACCCATGGTTTTTTTGGGTTTTTTTTT | |
| | | TGGAATAAAAAAATTTGGGGTTTGGGATACCC | |
| | | CCAAAAAATCAAAAAATATTTTTTGTAAAATA | |
| | | AAAGGGGCCCTTCCCTGAATTTTCCAAAACCC | |
| | | CCCCCTCTTTTTTTTTTTTGAAATTTCCCCCCCT | |
| | | CTTTTCCCAAAAGTCTTTTTCTTTTGAAAAAAA | |
| | | TAAATTTTTCAAAAAAATTTTTTAAAAGGCCCC | |
| | | CTTTTTAAGGGTTTTTCCTTTTGGGTTCTTTTTC | |
| | | CTTCCCTTTCCCACCACCCCTCCCCCTGGGGGG | |
| | | TTCCCTCCCTTTTTTTTTTTTTTTCCTGGGAAAC | |
| | | CCTTTCCCACCCTTATTTTTCCTTCTTTGGGGGG | |
| | | GGATAAACCCCCACCAGGGCCCCCCCCTTTTCC | |
| | | AGGCACCGGTTTTCCCGGGGAAAAAGGCCCCC | |
| | | CCCCCCCCCCCGTCCCCCCTTATTTCAAAAAGG | |
| | | AAATGCAAAGAAGAGAGAAAAACTCTCTTCGG | |
| | | GGCCCTCCACAAAAGGGGTCTACAAATACACA | |
| | | CCGATTGAATAGTTTAGGTTTAGAAAATGATA | |
| | | TATTTGGAATAACAATTTTTTTTAATTAAGAAA | |
| | | AAAAAGAAGGATAAAAATGATATTATAAAAGT | |
| | | TTCATTAAAGCCTATAATATTCCCACATAAATT | |
| | | TAAAAACGTCTTCTCCAAGAAGATATCTTATTG | |
| | | CTGCCTCTATATTAAGCTTTTTTTTTTTTTTTTTT | |
| | | TCCCTTCATTAACATCAAAAGGTCTTTGGAGAA | |
| | | AATGATGCAACGACCTGCAGAATTAAGAAGTT | |
| | | AATGATGTGCAAAATCATAACTTTCTTCTATTT | |
| | | TTATTAACAATAAAATTTTTTTCTTAACACTAC | |
| | | TAATCCAAATATTCACTTCAAAACTAATATTTG | |
| | | TTATTTTATTAATTTTTAAAAATATAATAATTT | |
| | | AATATCTACACATTACTTATTTTATCAAAATTT | |
| | | TCAAAGGTTCATGTGAAAATTTTATACTTTTAT | |
| | | CAATTTCCATCATTTTTCAAATTTTAAAACAAT | |
| | | TTTAATTTATATGCAAATTTCTACAGATATTTC | |
| | | CATAAAACTCGAAAATCAGTGAGGGTAACAAA | |
| | | GTCAATTTTTTTAACATTGGGTATATGTATATA | |
| | | TTTGAGGGAATTGTCAGTGAGAAGAATACATG | |
| | | GTAGGAAATGACACACGTCCCAAGCAACAGAT | |
| | | GGATGGTTGAGAATGAACCCGGTCATTGAATT | |
| | | GACATTAGAAAATGTTCTAAAATATATTTTTAT | |
| | | CGACGTTACTGATCAGAAACAAAGTCCTCCGC | |
| | | GAGCCAGAGGCATATCCTAATGATTGTACCTTT | |
| | | CCTCACTGACAATTCCCATTAATTAGCTGCTGC | |
| | | CACTGCATAGCGGCTATAATATAATGGTAGAG | |
| | | GACCCATGGAGCTTTCCTTTTCAGTGAACATGG | |
| | | GTTAGTCGACAAAAGAAAATGTTCAAGTTGAA | |
| | | AGAGGAGCGGTGGCCCTCAAAGGTTCCCATCA | |
| | | CTTGGTTGCTTTTTGTCTAAGGAAACAGTGGTA | |
| | | TCAGAATCCAAATCTTCTACGTAATGTCCCATT | |
| | | CATCCTACCAATGTCCATATGAATTCCTCTGGA | |
| | | CGTTAAAAAATGGTTGCACGTGGTTGCCTTCA | |
| | | GGATCACACCAGTTTATACATTTGGACCACAA | |
| | | AATAGAGATTGTTCATCAAGGATACTAGTCAG | |
| | | CAATTAATTCCTAAATATCTCTTATGACACATG | |
| | | TAGGGACCCCTCCCTCTGGGAAACACGTGGCA | |
| | | CGCACCTCACAGTGACACGCAGCACGTGTTAT | |
| | | CAGCCGGACCATCATCATCCGGATTCCCTTAA | |
| | | GGATACGCATGATGATGGTTCTCCTATCCGGA | |
| | | CCGCCTCAAGGAAAAGCACATGACGTTTCAGC | |
| | | TTCTTCTGTCCAAGGAAGAGCAAACGACGCTG | |
| | | ACAGAGCATAGACATCCGGACAACCTTCATAA | |
| | | TGTATCCGCTCCACTATACAATCCGGATAGTCA | |
| | | GCATGTGACCATCCGGATTTAATCGTCCGGATC | |
| | | ATCAATTAAAGTAAAGCAAGTCTTACACGCTA | |
| | | TCACGACAACCAGCCATGGCCCACGTCCCATC | |
| | | ATCTGCAGAGTGAAAGGACGGGTCGAGGTGAC | |
| | | AACAAGTCACTTCCCACGATCATTCTACATGAT | |
| | | CATTTCCCACGATATCTAGACAGCAGCATCAC | |
| | | CTACCACGGTTTCTGACAGCCGCCAGTAGGGT | |
| | | GGCGATGACCATGCTGCCTCCGAATGTCATCA | |
| | | TGACAAACATAAAATATCTCCTCGCCATTAAT | |
| | | GAGAGGAACAGTACCCCTGAAGCTGTATATAT | |
| | | ATGCCTTCGCACGAAGAAGAAGGGGATCCTCC | |
| | | TGGTAACTTCTTAATACCTGGTAAAAGGCCAA | |
| | | CTGATTTATATTCCTCTCTCTGACCATGGCTAA | |
| | | CAAAACCATCGGAGGATGCGTCCGGACACCCT | |
| | | GTCCGGATGCCTTCTTGCAGGAATGACGACTG | |
| | | GATCAAAAACCTTTATGAGTTGAGATCACGCG | |
| | | TCCATCCATCTGGTTACTACGTGGACCGCCAAA | |
| | | GACGCGAGGTAACAACATTGGCGCTGTCTGTG | |
| | | GGAACTTTTCTTTACTTTGATTCAGTCACTAAA | |
| | | AACAATGGCCACACCATCCCAAAGTCATTCAT | |
| | | CTGGTAGGGGAGAGGATGATAATTTTGAATGG | |
| | | CGCCTAGCCATCGAAAGGAGACAGTTGGCAAG | |
| | | CGAAAGACAGCTAAAAGCTCTCCTCCAGGAGA | |
| | | CAGAAAGATTGAGAGAAGAAAACGTTGTGTTA | |
| | | CGCATCCAGGCTTCAACATCAGAGCCTCCTCG | |
| | | ACTTCAACATTCGAGGGGCCAAGTAGCAAACT | |
| | | CAAGGCCTCAGCAAGAACCAGAGTCAATATAC | |
| | | CCTGGGACAACAGGGGCAATCCCAGGGGCATG | |
| | | CAACGTGAGGCCTCATGAGCCATGCACGCCTA | |
| | | TGCCTTGAGCTCCCCGTGAGGAAAGCTCAGAC | |
| | | TCTACTCACTTTTCAGCAAAAAGGCAACGTGA | |
| | | TAAAAAGTCGCGGTTGTCAAATTCGATGCGCG | |
| | | CAAGACTAGGCCCGCAAGAACCTAGAAGATCA | |
| | | AGGCCACCTATGGCCACAACCTGGGCACCACA | |
| | | CCCTGACCCCATGGCAACCCCCATGGTGCAAA | |
| | | ACGATCACCCGCATCGTGACCCCATAGTCACC | |
| | | CCTGTGATGCGGAACGTTCATCTGCACCTAGC | |
| | | GGAATAGCCAACTGGGAGAAACATCCCAAACG | |
| | | GGCCACCCGTTGGCTCCATCAGCAAAAGGCTG | |
| | | GATGACATGCTCTCCACGCCTTTCTGCTCTCAT | |
| | | ATCATTAATTATGAGCCCCCAAGGGGATTCCTC | |
| | | GTACCTAAATTTTCCACATACGATGGAACCAG | |
| | | CGATCCCTTCGATCACATCATGCATTATCGACA | |
| | | GCTCATGACGCTCGATATTGGCAACGATGCGC | |
| | | TGCTATGCAAAGTATTTCCCGCCAGTCTACAAG | |
| | | GACAGGCCCTCTCATGGTTTCATCGCCTACCTC | |
| | | CCAACTCTGTTGGCAATTTCAGGGACCTGTCCG | |
| | | AAGCTTTCGTGGGACAATACCTGTGTTCCGCCC | |
| | | GACACAAGCAAAATATCAGCACCTTGCAGAAC | |
| | | ATAAAAATGCAAGATAACGAATCCTTAAGGGA | |
| | | ATTTGTGAAGCGGTTTGGCCAAGCCGTACTTCA | |
| | | AGTGGAAGCTTGCAGTATGGATGCTGTTCTAC | |
| | | AGATCTTCAAACGAAGCATCTGTCCAGGCACT | |
| | | CCATTTTTCGAATCACTGGCTAAAAAGCCTCCT | |
| | | ACTACGATGGACGACTTATTCAGACGAGCCAA | |
| | | CAAATATTCAATGCTCGAAGATGACGTACGTG | |
| | | CAGCCACCCAGCAAGTTTTGGTTGCCGGACGG | |
| | | CCATCCGGAAGTAATACGGAGAGAAATACCAA | |
| | | ACCTCCGGATCGGCCAAAGCCGTCTGACCGAA | |
| | | GGCAGGAAGGGCCAAGTCGCCCGAAAATGCC | |
| | | GCCTCTCACACCTCTTTCTATATCATATGAGAA | |
| | | ACTTCTCCCAATGATCCAAGGCTTGTCCGACTT | |
| | | CAAGTGGCCTAGACCTATTGCAACGGACCCAT | |
| | | CTACAAGGGATCGTAGCAGGAGATGTGCCTTC | |
| | | CACAAAGATCATGGCCATACAACAGAGACGTG | |
| | | CCGATCTTTCCAGTATTTGGTCGAAAGACTCAT | |
| | | AAAAGCTGGGCATTTGAAGCAATACCTCCGCA | |
| | | CGGATACGGGGGGTAGGGACGTTTCCCAGCAC | |
| | | AACAACCCTGGAGCCCCTAGGGCCCCAGTTGC | |
| | | CACCAAGGCCGTTATCAACTATATTAACGGAG | |
| | | GACCATCTGACGAGGAGTATGACTCCAGACGG | |
| | | AAAAGGCAAAAATTGTTGCGGGCTGCATCAAT | |
| | | ACGCGAACGCATTAATTCCATCCGGCCGGGTC | |
| | | TAACTGGAGAGGGTCCTCGCCCCATAGATGGA | |
| | | ACAATAATTTTTCCACCAGTAGATCCCACCCGG | |
| | | ACACTACAACCACATCGCGACGCCCTCATCCT | |
| | | GTCCCTAGAAATTGGGGACTTCGATGTGCGGC | |
| | | GTATCTTAGTTGACCCAGGCAGTTCAGCCGAC | |
| | | CTGGTGCAAGCATCAGTCGTTGGCCACATGGG | |
| | | ACATAGTCTCACGGGCCTTGAAAACCCCGGAC | |
| | | GAATCTTATCCGGATTCAACGGATCATCAACC | |
| | | ACATCCTTGGGAGATATCATACTGCCGGTCCA | |
| | | AGCTGGCCCAGTCACTCTCAATGTACAGTTTTC | |
| | | AGTAGTACAGGAGCTATCACCCTTCAATATCA | |
| | | TTTTGGGGCGCACATGGCTCCACTACATGAAA | |
| | | GCTATCCCCTCTACATACCATCAAATGGTGATT | |
| | | TTTCTTACCCACGATGGTCAAATCGACTTATAT | |
| | | GGCAGCCAATTAGCCGCTCGCCAGTGCTATCA | |
| | | GATAGCACGTGAAGCAGGGGCCAACCAGGAG | |
| | | GATGCATCTCCCCCTGAACCCAGCATTGCACG | |
| | | CGACCAATAGCAATTATTGGGTCCGACGGACA | |
| | | AAGATCCCCCGGCAGCAGATCCCTTACAAACA | |
| | | ATCCAAATTTCGGAGGAAAGCGATCACCTCAC | |
| | | GAACATCAGTTCCCTCATGACACAAGAAGAGA | |
| | | CTCAGGGCATGCAAAATATCCTCAGACAGAAC | |
| | | CATGACATCTTCGCGTGGGCACATTCTGACATG | |
| | | AAGGGAATTCATCCCTCCATTACATCTCACAG | |
| | | GCTTAACGTCGTTTCAACTGCCAGACCCGTCCG | |
| | | ACAGAGGATTAGGCGCTTCCACCCAGATAGAC | |
| | | AAAGAGTCATCCGGAACGAGATTGACAAATTG | |
| | | CTCGAAGCCGGATTCATCAGAGAAGTTTCTTAT | |
| | | CCGGATTGGTTGGCAAACGTAGTCGTGGTACC | |
| | | CAAAAAAGAAGGAAAATGGCGAGTTTGTGTAG | |
| | | ATTACACCAATCTGAACAATGCGTGTCCAAAA | |
| | | GACAGTTTTCCCTTGCCGCGAATAGATCAGATT | |
| | | GTGGATTCCACTTCCGGGCAAGGGATGCTCTCT | |
| | | TTCTTGGATGCCTTCTCCGGATATCATCAAATC | |
| | | CCCATGTCCCCGGATGACGAAGAAAAAATAGC | |
| | | ATTCATAACACCACACGACCTCTATTGTTATAA | |
| | | AGTCATGCCATTCGGACTCAAAAATGCTGGCG | |
| | | CCACTTATCAAAGATTGATGACTAAAATCTTCA | |
| | | AACCTCTGATAGGCCACTCAGTAGAGGTTTAT | |
| | | ATTGACGATATCGTGGTTAAAAGCAAAACTCG | |
| | | GGAGCAGCATATCCTTCATTTGCAAGAGGTTTT | |
| | | TTACCTCTTACGAAGGTATGGCATGAAGCTAA | |
| | | ATCCTTCTAAATGCGCCTTTGGCGTAAGTGCCA | |
| | | GAAAATTTCTAGGATTTATGGTCAGCCAAAGG | |
| | | GGCATAGAAGTCAGCCCGGATCAAGTCAAAGC | |
| | | AGTCATGGAAACACCCCCTCCCAGGAACAAGA | |
| | | AAGAGTTACAGCGCCTCACAGGCAAACTCGTT | |
| | | GCATTAGGGCGTTTCATAGCCCGCTTCATTGAT | |
| | | GAGTTGCGACCCTTCTTCTTGGCGATACGAAA | |
| | | AGCTGGAACGCACGGGTGGACGGACAATTGCC | |
| | | AAAACGCTTTGGAAAGAATTAAACATTATCTT | |
| | | ATGCAACCACCCATCTTGAGCAGCCCCATCCC | |
| | | AAAGGAGAAATTATACATGTATCTAGCAGTGT | |
| | | CAGAATGGGCAATCAGTGCCGTTCTATTCCGCT | |
| | | GCCCATCACCCAAGGAGCAGAAGCCCATCTAC | |
| | | TATGTCAGCAGGGCATTGGCAGACGTAGAAAC | |
| | | CAGATATTCAAAAATGGAGCTAATATCCTTAG | |
| | | CCCTTCGAAGCGCTGCCCAAAAGCTCCGGCCC | |
| | | TATTTTCAAGCCCACCCGGTGATCGTGTTGACT | |
| | | GATCAACCCCTTCGCAACATTCTGCACAAGCC | |
| | | AGATTTAACCGGGAGAATGCTACAATGGGCCA | |
| | | TCGAATTAAGCGAGTTTGGGATTGAATTCCAA | |
| | | CCAAGGTTGTCCATGAAAGGCCAGGTAATGGC | |
| | | TGACTTCGTGCTAGAATATTCCCGAAAACCCG | |
| | | GCCAACATGAAGGATCAAGGAAAAAAGAATG | |
| | | GTGGACTCTGCGAGTTGACGGAGCCTCACGTT | |
| | | CATCAGGCTCTGGAGTCGGGCTCTTATTGCAGT | |
| | | CCCCAACTGGGGAACATTTGGAACAAGCCATC | |
| | | CGGCTGGGATTCTCCGCCTCTAACAATGAAGC | |
| | | AGAATATGAGGCTATCTTGTCCGGATTGGACC | |
| | | TCGCCCTTGCTCTATCCGTCTCCAAACTCCGGA | |
| | | TCTTCAGCGACTCACAACTCGTGGTAAAACAT | |
| | | GTCCAGGAGGAATATGAGGCTAAGGACGCACG | |
| | | CATGGCGCGATACTTAGCTAAAGTAAGAAACA | |
| | | CCTTGCAGCAATTCACTGAATGGACAATCGAA | |
| | | AAAATCAAGCGAGCTGACAATAGGCGCGCTGA | |
| | | CGCTTTGGCCGGCATAGCTGCCTCCCTCTCTAT | |
| | | CAAGGAAGCCATTCTACTGCCCATACATGTGC | |
| | | AAACCAACCCCTCTGTCTCAGAAATTTCAATCT | |
| | | GCAGTACCACTGAGGCACCCCAAGCGGACGAC | |
| | | CAAGAATGGATGAATGATATCACAGAATATAT | |
| | | CCGGACAGGAACTCTACCCGGAGATCCCAAAC | |
| | | AGGCACATAAAGTCCGGGTGCAAGCTGCCCGT | |
| | | TTTACCTTAATTGGGGGGCACCTGTACAAGCG | |
| | | ATCCTTCACAGGACCATATCTTCGGTGCCTAGG | |
| | | GCATTCAGAAGCTCAATATGTGTTGGCTGAGTT | |
| | | ACATGAGGGAATATACGGAAATCATTCGGGAG | |
| | | GACGATCTCTGGCACATAGAGCCCATTCACAA | |
| | | GGATACTATTGGCCAACAATGAAGAAAGAGGC | |
| | | GGCAGCATATGTCAAAAGATGTGATAAATGTC | |
| | | AAAGGTATGCTCCCATCCCACATATGCCATCA | |
| | | ACAACGTTAAAATCAATCTCAGGTCCATGGCC | |
| | | CTTCGCACAGTGGGGCATGGACATAGTGAGAC | |
| | | CTCTCCCAACTGCACCTGCCCAAAAGAAATTTT | |
| | | TGCTTGTTGCCACAGATTATTTCAGTAAGTGGG | |
| | | TGGAAGCTGAAGCATATGCTAGCACCAAAGAT | |
| | | AAGGATGTCACCAAGTTTGTATGGAAAAATAT | |
| | | TATCTGCCGTTTTGGAATTCCCCAAACCATCAT | |
| | | AGCCGACAATGGTCCACAATTCGATAGCATTG | |
| | | CATTTAGAAATTTCTGTTCAGAGCTAAACATCC | |
| | | GGAACTCATACTCCACACCACGATATCCTCAA | |
| | | AGCAATGGGCAAGCAGAAGCCACAAACAAGA | |
| | | CCCTAATCACTGCCTTAAAGAAAAGGCTTGAG | |
| | | CAAGCCAAAGGAAAATGGGTCGAGGAGCTGC | |
| | | CCGGCGTCCTATGGGCCTACCGAACTACACCC | |
| | | GGACGGCCAACAGGAAATACTCCTTTCGCTCT | |
| | | TGCATATGGAATGGACGCCGTCATTCCTATCG | |
| | | AAATAGGTCTCCCTACTATCTGGACCAACGCA | |
| | | GCAAAACAAAGTGATGCAAACATGCAATTAGG | |
| | | AAGAAATTTGGATTGGACGGATGAAGTGAGGG | |
| | | AAAGCGCGTCCATCCGGATGGCAGATTATCAA | |
| | | CAAAGGGCATCAGCACATTACAATCGCAAAGT | |
| | | GAGACCAAGAAGCTTAAAAAATGGTACGCTAG | |
| | | TCCTTAGAAAATTTTTTGAAAATACTACTGAAG | |
| | | TAGGTGCAGGGAAGTTCCAAGCCAATTGGGAA | |
| | | GGACCTTATATAGTGTCTAAAGCAAGTGACAA | |
| | | TGGAGCCTATCATTTACAAAAGCTAGACGGAA | |
| | | CTCCATTACTTAGACCATGGAATGTGTCTAATT | |
| | | TAAAGCAGTATTATCAATAAGAAAGCCAAGGC | |
| | | AAGTTCAAATGAGAAAAGAAGTATTTTTATTG | |
| | | ACATAGCTAAAAATATGTACAAAAGAGGTCTC | |
| | | CGGACCACAAAAATACAAAAGGAAGATTACA | |
| | | GAAGAAATTGCAAAAAGAGAAACTGTCAGGG | |
| | | AGCAGGCTTGTCATAAAGTTTCTTTTCTTCACC | |
| | | CGGAGGAATTGAAGGCACGTCCCGCTTGATAC | |
| | | CGTTCTTCTTCATGCAGCACCGATACCCAAAGA | |
| | | TGAACGTATCATCCACCTGTTTTTTATATTCTG | |
| | | CTTCAATTTCCTCCCTCTCTACAGCAAACTCCC | |
| | | GTTCTAGCTCCTCTTTTTGTGCAACCAAGCGCA | |
| | | GCTTTAAATCTTCCCTCTGTTTCTTTTCAATAGA | |
| | | AACTTCTGTCCGGAGTTGCCTCACCTCCCCCCT | |
| | | CAGTTGGGTTATCTCATCTGCTGCCTCATGCAG | |
| | | GCGGTCGGCCGCTGATTCCTCCTGACTCTTTGC | |
| | | CTCAGCCAAATCCACCCGGAGGGCTTCATTTTC | |
| | | TTTTCGAATGGTGGATAAGCCAGCTTCAGCCTC | |
| | | TTCCAGTTTCAGGCGCAGTTGGTTTTCGCTTTC | |
| | | TTTATGCCAAGAGGCAAAGATCCTCATATAAT | |
| | | CAGTGGTCCGCAGCAAATCAAAAAAGAGATCG | |
| | | TGGTGTTGGGCCATGCCGCGGAGACCACTCAC | |
| | | CAGCTGACACACAAACAAGCAATGCAAAGTTA | |
| | | CAAATCATACCACAACAACACAACAAAATGAT | |
| | | AAAACAATCAAAAAGCACGAAAAACAGTTTTA | |
| | | CCATTTCCACATTTTCGAACATCTGGGCTGAGG | |
| | | GCATGATATCAGGCGAGCCGGGTGGAATCTCT | |
| | | TTCAGCTTATCTTCCAACTCCGCATAACTAAAA | |
| | | GAGTTGTCGGAGGAGCAAGATGCGTCATTAAC | |
| | | AGGTCCACCCCCGGATGAAGCAGCAGAAGGCG | |
| | | ATTTTTGTTCCGAGTGAGGGGCCCCAACATTTC | |
| | | CGGCCGGATGGGCCTCCCCAGGTGCAACCTCA | |
| | | GCCGGGACCACCATCGGGACGGATGGAGCCTC | |
| | | AGTTACCATCTCCACTTCGCCCCCCTCCGGATG | |
| | | AGCGTCGTGGGCAGATGAGCAACTGACTTCAA | |
| | | TCTCTTGCTACCGCTCTTGAAGCTGCCCGTGGA | |
| | | GTCCGGACTTCAGATCGCGCACCGAGCGAGGC | |
| | | TTCTTTGAGGATGGCCCCGTCACTAGAACAAG | |
| | | AGCCAGCAGGTTCGGATCGTCAGAGGGCTGAC | |
| | | TCTGGCTTTCTGCTCCCACTTCCTCCATGGGGG | |
| | | TCGCGCAAATGGCCTCAGCTGCATCGGCATCC | |
| | | GGATTGCAGGAGTCCGGATGGTTGACAGACGC | |
| | | AGCTTCCTCAGCCAAATTCACTAGACGCGCAA | |
| | | CTGCTGCCAAGGAGGTGCTTGAGTGGTTCAGC | |
| | | CCCGCAAGGTGTCCGGACCCGCTTGAGATGGA | |
| | | ATGAGGGGCAGCGTTCACTGGCTCCTCTATCAT | |
| | | TACCTCCGCCTCGTGAATAATGGGCGAAGGAG | |
| | | CAAATTCCATGGGAGAACTGGGCTTCTTCAAG | |
| | | TCCTTCCCATTTTTCTTCACCAGCTTCCTCTTTT | |
| | | TCGCTGGAATTTTCTCTGGAGGAGAGTCTGCGT | |
| | | CACGATTCTGTCCGGGAGCCTTCCGAAGGGTC | |
| | | CCTTCGTTCTTCTTCTTCTCCCGATCCTCCAGGA | |
| | | GAAGTCGACATTTTTTAACGTCAGCCTCTTTTA | |
| | | ACGCCTCGTAAATCGGGAAATCCTTCACAGTA | |
| | | TAATGCTCCCCAGGAACAACTTCCTTTGGCATC | |
| | | TTCCTGGGGAGAATGTTGATGACATATTCTCGG | |
| | | GGCTCCCGGACGACCGCCGTCAGGTTCCGCGC | |
| | | GGTCAGCAGCGTCTTGCACTGCCTCTCCTTGGC | |
| | | GTCTATCTCGAACAATTTGCTGAGACAGCCAA | |
| | | AGGACGCCTTCTCCACCCAGTCCACAAGGTGG | |
| | | CCCCTCAATTCCGGACCTACATTAACAGCAAC | |
| | | AAAAGATGAGAATGCCATCCGGAAGAGTCAAC | |
| | | AAAAAATCAACAAAGTAAAAACCGGACAATA | |
| | | GTCACAAAGCAAACCTACCCGGAACCACCAAG | |
| | | GAGTAGTTTGGAGAGAAAGGCCTCGCCGGATG | |
| | | CTCCAAAAGCCCCGCCCATGCACCCCGGACCA | |
| | | CCACGTGTCCCGTCGCCCCTCCCTTTGTCGAAT | |
| | | CTGGCAGCTCCGTCACCAGTTGGAGGGAGGGC | |
| | | AGGTGTGCGGACATGCTGAAGATGTCATTTTT | |
| | | GACCTTCTTCAAAGAATACACAAAAAACACCT | |
| | | CCAGCAGCGTGAGATCGAGGTTGTACAGCATG | |
| | | TTTATGATGCTGCATCCCATCAGCACCCGGACG | |
| | | ATGTTGGGATGAATAAAGACGGGTGGAATCTG | |
| | | GGTGAAGTGGAGGAACTCCTTGAACAACGCCA | |
| | | GCAGAGGGAACCGGAGCCCCGCGTTGAATTGT | |
| | | TCCTTCGAGAAGATGATAGTTTTTTCTTCTGCT | |
| | | TTCTCAGAAGGAACAAGCACCCTCCCATTCAG | |
| | | CAATTCGATAGCCACGTCATTTGGGATACAGA | |
| | | ACCGTTCCCGGAATTCCTTCGCGTCCAACTTAT | |
| | | CTATCGCTTTTCCACGAACCTCGGTACCCCGAG | |
| | | AAGATGAAACAGTCTTTTTTGCAGCCATTTCTT | |
| | | ACCACAAACACAAGCCCAAAACTACACACAAG | |
| | | AAGCGCAATGGGTAAAACACAAGTAACCCAG | |
| | | AAAATACACAAACCCTAACCCGAACAAATCAG | |
| | | AAACCAAAACCAACAAAACGCAACAAAAGGA | |
| | | ACAGCAATAGCAAAAGTACACGTACCAAATGA | |
| | | AGCTCTGAAGAAGAAAGGTCGTCGTTTCGAAT | |
| | | ACAAGAACACCAGCAGCAAACAAATGTCGCG | |
| | | ACACAAAATCACCGGTACAAAAGTGTTTTCGA | |
| | | GTTTCTTTACTCAGAAAAAGCAAAGGACGCAA | |
| | | CAAAAGCAAGAAGAAGAAAGGATCTCAGGAA | |
| | | AATGCAGTAGAAAAAATGAAAAAGAAGGTAC | |
| | | AGTACGATATTTATAGGGGGACCAGCCCTCGG | |
| | | AAAGCCAAACGTCCAAACAGCGCTATCATTGA | |
| | | AACAACATGCTGCCTGGGAATTCCCAGAGTCG | |
| | | CCGGCTCATCATAAATGCCTTTTATGGCTTCCG | |
| | | CACCCCCTCTCGCCACGTGGCTCAGTCAGACG | |
| | | AAGAGACCTCTTCAAAATTCAAAAAGCCAGTT | |
| | | ATTTTTTAACCCGCCCATTTTTTGGCAAAATAG | |
| | | GCAAGTTAAAAAGGGGGGCAATGTAGGGACC | |
| | | CCTCCCTCTGGGAAACACGTGGCACGCACCTC | |
| | | ACAGTGACACGCAGCACGTGTTATCAGCCGGA | |
| | | CCATCATCATCCGGATTCCCTTAAGGATACGCA | |
| | | TGATGATGGTTCTCCTATCCGGACCGCCTCAAG | |
| | | GAAAAGCACATGACGTTTCAGCTTCTTCTGTCC | |
| | | AAGGAAGAGCAAACGACGCTGACAGAGCATA | |
| | | GACATCCGGACAACCTTCATAATGTATCTGCTC | |
| | | CACTATACAATCCGGATAGTCAGCATGTGACC | |
| | | ATCCGGATTTAATCGTCCGGATCATCAATTAAA | |
| | | GTAAAGCAAGTCTTACACGCTATCACGACAAC | |
| | | CAGCCATGGCCCACATCCCATCATCTGCAGAG | |
| | | TGAAAGGACGGGTCGAGGTGACAACAAGTCAC | |
| | | TTCCCACGATCATTCTACATGATCATTTCCCAC | |
| | | GATATCTAGACAGCAGCATCACCTACCACGGT | |
| | | TTCTGACAGCCGCCAGTAGGGTGGCGATGACC | |
| | | ATGCTGCCTCCGAATGTCATCATGACAAACAT | |
| | | AAAATATCTCCTCGCCATTAATGAGAGGAACA | |
| | | GTACCCCTGAAGCTGTATATATATGCCTTCGCA | |
| | | CGAAGAAGAAGGGGATCCTCCTGGTAACTTCT | |
| | | TAATACCTGGTAAAAGGCCAACTAATTTATATT | |
| | | CCTCTCTCTAACCATGGCTAACAAAACCATCG | |
| | | GAGGATGCGTCCAGACACCCTGTCCGGATGCC | |
| | | TTCTTGCAGGAATGACGACTGGATCAAAAACC | |
| | | TTTATGAGTTGAGATCACGCGTCCATCCATCTG | |
| | | GTTACTACGTGGACCGCCAAAGACGCGAGGTA | |
| | | ACAACAACACACACCCTTTGTCCATGAACTCC | |
| | | AGCGCATTTGGAAGCCAGTAATGCACCATAAG | |
| | | AAACGTGTCGAATAAACCAATTAGGGGTCTGG | |
| | | TGTCCGAGTCATGAGATAGAACAGGTTCGAGG | |
| | | TTGTTATATATCAATCAATAATTAGAGAAGGA | |
| | | GCCGGTCTCTTGTGTTGAGTTGACTCGATGGAG | |
| | | AGCTTAGGAGTTAGAAAGGGTGCATGGATCCA | |
| | | AGAAGAGGATGTTCTCCTGAGGAAATGCATTG | |
| | | AGAAATATGGAGAAGGAAAGTGGCATCTGGTT | |
| | | CCCCTCCGAGCAGGTAACATGAAAGAGAAAGG | |
| | | GATCAGTATTTATTTGTGTTTTTTTACTTCTGTT | |
| | | TTGCTTAAAGAGTTTCATTTTCTTGAGTTTGCA | |
| | | GGGTTGAATAGATGCCGAAAAAGCTGCAGGTT | |
| | | GAGATGGCTCAATTATTTGAAGCCGGATATCA | |
| | | AGAGAGGAGAGTTTGCATTAGACGAGGTTGAT | |
| | | CTCATGATTAGGCTTCACAATTTGTTGGGGAAC | |
| | | AGGCAAGTCTATAATAACTCAAGTACTAGCTT | |
| | | GATAATGATATTATATTAGTTCTGAAGCTGTTC | |
| | | AGAACTTACAAAAGAGCTGTTCAGTTGATACT | |
| | | TTGTCTGATGTTGTGCGTGTATAGATGGTCCTT | |
| | | GATTGCGGGTAGGCTTCCAGGGAGGACTGCTA | |
| | | ATGATGTCAAGAACTATTGGCATAGTCACCAC | |
| | | TTCAAAAAGGAGGTTCAGTTCCAGGAAGAAGG | |
| | | GAGAGATAAACCCCAAACACATTCTAAAACCA | |
| | | AAGCTATAAAGCCTCACCCTCACAAGTTCTCC | |
| | | AAAGCCTTGCCAAGGTTTGAACTAAAAACTAC | |
| | | AGCTGTGGATACTTTTGACACACAAGTCAGTA | |
| | | CTTCCAGGAAGCCATCATCCACTTCACCACAA | |
| | | CCGAATGATGACATCATATGGTGGGAAAGCCT | |
| | | GTTAGCTGAGCATGCTCAAATGGATCAAGAAA | |
| | | CTGACTTTTCGGCTTCTGGAGAGATGCTTATCG | |
| | | CAAGCCTCAGGACAGAAGAAACTGCAACACA | |
| | | GAAAAAGGGACCCATGGATGGTATGATTGAAC | |
| | | AAATCCAGGGAGGTGAGGGTGATTTTCCATTT | |
| | | GATGTGGGCTTCTGGGATACACCCAACACACA | |
| | | AGTAAATCACTTGATCTGATTTAGACCATTICA | |
| | | ATCTTAATGTTTAAGTTCAAAAGTTGTTCTTGC | |
| | | ATTCATGCTCTACTCCCTAATCATTTTATGTTCC | |
| | | AAAGTTGTTAATCTTAGAAATAAAAACAAAAA | |
| | | TAATAAATATATCAATATGTATAATGGTGGAT | |
| | | AGGGATGTGAGCAGTGGATGCAAACTCAAGCA | |
| | | TTGAATGAATGGTTAAGATTAATGAACCCAAT | |
| | | CATTTGAAGTGACAAAAGAAAGACATTTTAAA | |
| | | AGATTCTTAACAATATGAAAAAGTTGTTTCATA | |
| | | TTTCTATCGGTTTCCAACCATTAATTAGACGGA | |
| | | TGGACCATTGAAATATCTCATGCATATTGAATT | |
| | | AATAAAACAAGAAATTTCCACATAGTTACATC | |
| | | GGTAAAAAGAAAATAAGTTGAAAATGGAAAA | |
| | | TTATGGTGCCAATTTTGGATATGATAGAATGAC | |
| | | TTCAATGTTTGCCTATAATGGTTAATTATAATA | |
| | | TTAACACATCTCTCTTTCTAGCTTGTTAGTTAA | |
| | | TATTGACACATCTCTATTTTTAGGGCCCTATTT | |
| | | GGTAATTGGTAATTGTTTTTTAAAACAATTGTA | |
| | | AAAAATAGTTTTTAAAAACTATTTTATAGTGT | |
| Hybrid 1 | Hypothetical | tgtagggacccctccctctgggaaacacgtggcacgcacctcacagtgaca | 37 |
| | repair | cgcagcacgtgttatcagccggaccatcatcatccggattcccttaaggatac | |
| | outcome if | gcatgatgatggttctcctatccggaccgcctcaaggaaaagcacatgacgtt | |
| | editing with | tccaaacgacgctgacagagcatagacatccggacaaccttcataatgtatct | |
| | Guide 1 led | gctccactatacaatccggatagtcagcatgtgaccatccggatttaatcgtcc | |
| | to removal | ggatcatcaattaaagtaaagcaagtcttacacgctatcacgacaaccagcca | |
| | GRET1 | tggcccacatcccatcatctgcagagtgaaaggacgggtcgaggtgacaac | |
| | leaving | aagtcacttcccacgatcattctacatgatcatttcccacgatatctagacagca | |
| | partial 5′ and | gcatcacctaccacggtttctgacagccgccagtagggtggcgatgaccatg | |
| | 3′ TIR | ctgcctccgaatgtcatcatgacaaacataaaatatctcctcgccattaatgag | |
| | | aggaacagtacccctgaagctgtatatatatgccttcgcacgaagaagaagg | |
| | | ggatcctcctggtaacttcttaatacctggtaaaaggccaactaatttatattcct | |
| | | ctctctaaccatggctaacaaaaccatcggaggatgcgtccagacaccctgt | |
| | | ccggatgccttcttgcaggaatgacgactggatcaaaaacctttatgagttga | |
| | | gatcacgcgtccatccatctggttactacgtggaccgccaaagacgcgaggt | |
| | | aacaacAACACACACCCTTTGTCCATGAACTCCAG | |
| | | CGCATTTGGAAGCCAGTAATGCACCATAAGAA | |
| | | ACGTGTCGAATAAACCAATTAGGGGTCTGGTG | |
| | | TCCGAGTCATGAGATAGAACAGGTTCGAGGTT | |
| | | GTTATATATCAATCAATAATTAGAGAAGGAGC | |
| | | CGGTCTCTTGTGTTGAGTTGACTCGATGGAGAG | |
| | | CTTAGGAGTTAGAAAGGGTGCATGGATCCAAG | |
| | | AAGAGGATGTTCTCCTGAGGAAATGCATTGAG | |
| | | AAATATGGAGAAGGAAAGTGGCATCTGGTTCC | |
| | | CCTCCGAGCAGGTAACATGAAAGAGAAAGGG | |
| | | ATCAGTATTTATTTGTGTTTTTTTACTTCTGTTT | |
| | | TGCTTAAAGAGTTTCATTTTCTTGAGTTTGCAG | |
| | | GGTTGAATAGATGCCGAAAAAGCTGCAGGTTG | |
| | | AGATGGCTCAATTATTTGAAGCCGGATATCAA | |
| | | GAGAGGAGAGTTTGCATTAGACGAGGTTGATC | |
| | | TCATGATTAGGCTTCACAATTTGTTGGGGAACA | |
| | | GGCAAGTCTATAATAACTCAAGTACTAGCTTG | |
| | | ATAATGATATTATATTAGTTCTGAAGCTGTTCA | |
| | | GAACTTACAAAAGAGCTGTTCAGTTGATACTTT | |
| | | GTCTGATGTTGTGCGTGTATAGATGGTCCTTGA | |
| | | TTGCGGGTAGGCTTCCAGGGAGGACTGCTAAT | |
| | | GATGTCAAGAACTATTGGCATAGTCACCACTT | |
| | | CAAAAAGGAGGTTCAGTTCCAGGAAGAAGGG | |
| | | AGAGATAAACCCCAAACACATTCTAAAACCAA | |
| | | AGCTATAAAGCCTCACCCTCACAAGTTCTCCA | |
| | | AAGCCTTGCCAAGGTTTGAACTAAAAACTACA | |
| | | GCTGTGGATACTTTTGACACACAAGTCAGTACT | |
| | | TCCAGGAAGCCATCATCCACTTCACCACAACC | |
| | | GAATGATGACATCATATGGTGGGAAAGCCTGT | |
| | | TAGCTGAGCATGCTCAAATGGATCAAGAAACT | |
| | | GACTTTTCGGCTTCTGGAGAGATGCTTATCGCA | |
| | | AGCCTCAGGACAGAAGAAACTGCAACACAGA | |
| | | AAAAGGGACCCATGGATGGTATGATTGAACAA | |
| | | ATCCAGGGAGGTGAGGGTGATTTTCCATTTGA | |
| | | TGTGGGCTTCTGGGATACACCCAACACACAAG | |
| | | TAAATCACTTGATCTGATTTAGACCATTTCAAT | |
| | | CTTAATGTTTAAGTTCAAAAGTTGTTCTTGCAT | |
| | | TCATGCTCTACTCCCTAATCATTTTATGTTCCA | |
| | | AAGTTGTTAATCTTAGAAATAAAAACAAAAAT | |
| | | AATAAATATATCAATATGTATAATGGTGGATA | |
| | | GGGATGTGAGCAGTGGATGCAAACTCAAGCAT | |
| | | TGAATGAATGGTTAAGATTAATGAACCCAATC | |
| | | ATTTGAAGTGACAAAAGAAAGACATTTTAAAA | |
| | | GATTCTTAACAATATGAAAAAGTTGTTTCATAT | |
| | | TTCTATCGGTTTCCAACCATTAATTAGACGGAT | |
| | | GGACCATTGAAATATCTCATGCATATTGAATTA | |
| | | ATAAA | |
| Hybrid 2 | Hypothetical | tgtagggacccctccctctgggaaacacgtggcacgcacctcacagtgaca | 38 |
| | repair | cgcagcacgtgttatcagccggaccatcatcatccggattcccttaaggatac | |
| | outcome if | gcatgatgatggttctcctatccggaccgcctcaaggaaaagcacatgacgtt | |
| | editing with | tcagcttcttctgtccaaggaagagcaaacgacgctgacagagcatagacat | |
| | Guide 3 led | ccggacaaccttcataatgtatccgctccactatacaatccggatagtcagcat | |
| | to removal | gtgaccatccggatttaatcgtccggatcatcaattaaagtaaagcaagtctta | |
| | GRET1 | cacgctatcacgacaaccagccatggcccacgtcccatcatctgcagagtga | |
| | leaving | aaggacgggtcgaggtgacaacaagtcacttcccacgatcattctacatgat | |
| | partial 5′ and | catttccacctaccacggtttctgacagccgccagtagggtggcgatgaccat | |
| | 3′ TIR | gctgcctccgaatgtcatcatgacaaacataaaatatctcctcgccattaatga | |
| | | gaggaacagtacccctgaagctgtatatatatgccttcgcacgaagaagaag | |
| | | gggatcctcctggtaacttcttaatacctggtaaaaggccaactaatttatattc | |
| | | ctctctctaaccatggctaacaaaaccatcggaggatgcgtccagacaccct | |
| | | gtccggatgccttcttgcaggaatgacgactggatcaaaaacctttatgagttg | |
| | | agatcacgcgtccatccatctggttactacgtggaccgccaaagacgcgag | |
| | | gtaacaacAACACACACCCTTTGTCCATGAACTCC | |
| | | AGCGCATTTGGAAGCCAGTAATGCACCATAAG | |
| | | AAACGTGTCGAATAAACCAATTAGGGGTCTGG | |
| | | TGTCCGAGTCATGAGATAGAACAGGTTCGAGG | |
| | | TTGTTATATATCAATCAATAATTAGAGAAGGA | |
| | | GCCGGTCTCTTGTGTTGAGTTGACTCGATGGAG | |
| | | AGCTTAGGAGTTAGAAAGGGTGCATGGATCCA | |
| | | AGAAGAGGATGTTCTCCTGAGGAAATGCATTG | |
| | | AGAAATATGGAGAAGGAAAGTGGCATCTGGTT | |
| | | CCCCTCCGAGCAGGTAACATGAAAGAGAAAGG | |
| | | GATCAGTATTTATTTGTGTTTTTTTACTTCTGTT | |
| | | TTGCTTAAAGAGTTTCATTTTCTTGAGTTTGCA | |
| | | GGGTTGAATAGATGCCGAAAAAGCTGCAGGTT | |
| | | GAGATGGCTCAATTATTTGAAGCCGGATATCA | |
| | | AGAGAGGAGAGTTTGCATTAGACGAGGTTGAT | |
| | | CTCATGATTAGGCTTCACAATTTGTTGGGGAAC | |
| | | AGGCAAGTCTATAATAACTCAAGTACTAGCTT | |
| | | GATAATGATATTATATTAGTTCTGAAGCTGTTC | |
| | | AGAACTTACAAAAGAGCTGTTCAGTTGATACT | |
| | | TTGTCTGATGTTGTGCGTGTATAGATGGTCCTT | |
| | | GATTGCGGGTAGGCTTCCAGGGAGGACTGCTA | |
| | | ATGATGTCAAGAACTATTGGCATAGTCACCAC | |
| | | TTCAAAAAGGAGGTTCAGTTCCAGGAAGAAGG | |
| | | GAGAGATAAACCCCAAACACATTCTAAAACCA | |
| | | AAGCTATAAAGCCTCACCCTCACAAGTTCTCC | |
| | | AAAGCCTTGCCAAGGTTTGAACTAAAAACTAC | |
| | | AGCTGTGGATACTTTTGACACACAAGTCAGTA | |
| | | CTTCCAGGAAGCCATCATCCACTTCACCACAA | |
| | | CCGAATGATGACATCATATGGTGGGAAAGCCT | |
| | | GTTAGCTGAGCATGCTCAAATGGATCAAGAAA | |
| | | CTGACTTTTCGGCTTCTGGAGAGATGCTTATCG | |
| | | CAAGCCTCAGGACAGAAGAAACTGCAACACA | |
| | | GAAAAAGGGACCCATGGATGGTATGATTGAAC | |
| | | AAATCCAGGGAGGTGAGGGTGATTTTCCATTT | |
| | | GATGTGGGCTTCTGGGATACACCCAACACACA | |
| | | AGTAAATCACTTGATCTGATTTAGACCATTTCA | |
| | | ATCTTAATGTTTAAGTTCAAAAGTTGTTCTTGC | |
| | | ATTCATGCTCTACTCCCTAATCATTTTATGTTCC | |
| | | AAAGTTGTTAATCTTAGAAATAAAAACAAAAA | |
| | | TAATAAATATATCAATATGTATAATGGTGGAT | |
| | | AGGGATGTGAGCAGTGGATGCAAACTCAAGCA | |
| | | TTGAATGAATGGTTAAGATTAATGAACCCAAT | |
| | | CATTTGAAGTGACAAAAGAAAGACATTTTAAA | |
| | | AGATTCTTAACAATATGAAAAAGTTGTTTCATA | |
| | | TTTCTATCGGTTTCCAACCATTAATTAGACGGA | |
| | | TGGACCATTGAAATATCTCATGCATATTGAATT | |
| | | AATAAA | |
| Hybrid 3 | Hypothetical | tgtagggacccctccctctgggaaacacgtggcacgcacctcacagtgaca | 39 |
| | repair | cgcagcacgtgttatcagccggaccatcatcatccggattcccttaaggatac | |
| | outcome if | gcatgatgatggttctcctatccggaccgcctcaaggaaaagcacatgacgtt | |
| | editing with | tcagcttcttctgtccaaggaagagcaaacgacgctgacagagcatagacat | |
| | Guide 7 led | ccggacaaccttcataatgtatccgctccactatacaatccggatagtcagcat | |
| | to removal | gtgaccatccggatttaatcgtccggatcatcaattaaagtaaagcaagtctta | |
| | GRET1 | cacgctatcacgacaaccagccatggcccacgtcccatcatctgcagagtga | |
| | leaving | aaggacgggtcgaggtgacaacaagtcacttcccacgatcattctacatgat | |
| | partial 5′ and | catttcccacgatatctagacagcagcatcacctaccacggtttctgacagcc | |
| | 3′ TIR | gccagtagggtggcgatgaccatgctgcctccgaatgtcatcatgacaaaca | |
| | | taaaatatctcctcgccattaatgagaggaacagtacccctgaagctgtatata | |
| | | tatgccttcgcacgaagaagaaggggatcctcctggtaacttcttaatacctg | |
| | | gtaaaaggccaactgatttatattcctctctctgaccatggctaacaaaaccatc | |
| | | ggaggatgcgtccggacaccctgtccggatgccttcttgcaggaatgacga | |
| | | ctggatcaaaaacctttacatctggttactacgtggaccgccaaagacgcga | |
| | | ggtaacaacAACACACACCCTTTGTCCATGAACTCC | |
| | | AGCGCATTTGGAAGCCAGTAATGCACCATAAG | |
| | | AAACGTGTCGAATAAACCAATTAGGGGTCTGG | |
| | | TGTCCGAGTCATGAGATAGAACAGGTTCGAGG | |
| | | TTGTTATATATCAATCAATAATTAGAGAAGGA | |
| | | GCCGGTCTCTTGTGTTGAGTTGACTCGATGGAG | |
| | | AGCTTAGGAGTTAGAAAGGGTGCATGGATCCA | |
| | | AGAAGAGGATGTTCTCCTGAGGAAATGCATTG | |
| | | AGAAATATGGAGAAGGAAAGTGGCATCTGGTT | |
| | | CCCCTCCGAGCAGGTAACATGAAAGAGAAAGG | |
| | | GATCAGTATTTATTTGTGTTTTTTTACTTCTGTT | |
| | | TTGCTTAAAGAGTTTCATTTTCTTGAGTTTGCA | |
| | | GGGTTGAATAGATGCCGAAAAAGCTGCAGGTT | |
| | | GAGATGGCTCAATTATTTGAAGCCGGATATCA | |
| | | AGAGAGGAGAGTTTGCATTAGACGAGGTTGAT | |
| | | CTCATGATTAGGCTTCACAATTTGTTGGGGAAC | |
| | | AGGCAAGTCTATAATAACTCAAGTACTAGCTT | |
| | | GATAATGATATTATATTAGTTCTGAAGCTGTTC | |
| | | AGAACTTACAAAAGAGCTGTTCAGTTGATACT | |
| | | TTGTCTGATGTTGTGCGTGTATAGATGGTCCTT | |
| | | GATTGCGGGTAGGCTTCCAGGGAGGACTGCTA | |
| | | ATGATGTCAAGAACTATTGGCATAGTCACCAC | |
| | | TTCAAAAAGGAGGTTCAGTTCCAGGAAGAAGG | |
| | | GAGAGATAAACCCCAAACACATTCTAAAACCA | |
| | | AAGCTATAAAGCCTCACCCTCACAAGTTCTCC | |
| | | AAAGCCTTGCCAAGGTTTGAACTAAAAACTAC | |
| | | AGCTGTGGATACTTTTGACACACAAGTCAGTA | |
| | | CTTCCAGGAAGCCATCATCCACTTCACCACAA | |
| | | CCGAATGATGACATCATATGGTGGGAAAGCCT | |
| | | GTTAGCTGAGCATGCTCAAATGGATCAAGAAA | |
| | | CTGACTTTTCGGCTTCTGGAGAGATGCTTATCG | |
| | | CAAGCCTCAGGACAGAAGAAACTGCAACACA | |
| | | GAAAAAGGGACCCATGGATGGTATGATTGAAC | |
| | | AAATCCAGGGAGGTGAGGGTGATTTTCCATTT | |
| | | GATGTGGGCTTCTGGGATACACCCAACACACA | |
| | | AGTAAATCACTTGATCTGATTTAGACCATTTCA | |
| | | ATCTTAATGTTTAAGTTCAAAAGTTGTTCTTGC | |
| | | ATTCATGCTCTACTCCCTAATCATTTTATGTTCC | |
| | | AAAGTTGTTAATCTTAGAAATAAAAACAAAAA | |
| | | TAATAAATATATCAATATGTATAATGGTGGAT | |
| | | AGGGATGTGAGCAGTGGATGCAAACTCAAGCA | |
| | | TTGAATGAATGGTTAAGATTAATGAACCCAAT | |
| | | CATTTGAAGTGACAAAAGAAAGACATTTTAAA | |
| | | AGATTCTTAACAATATGAAAAAGTTGTTTCATA | |
| | | TTTCTATCGGTTTCCAACCATTAATTAGACGGA | |
| | | TGGACCATTGAAATATCTCATGCATATTGAATT | |
| | | AATAAA | |
| Full | Hypothetical | tgtagggacccctccctctgggaaacacgtggcacgcacctcacagtgaca | 40 |
| GRET1 | repair | cgcagcacgtgttatcagccggaccatcatcatccggattcccttaaggatac | |
| removal 1 | outcome if | gcatgatgatggttctcctatccggaccgcctcaaggaaaagcacatgacgtt | |
| | editing with | tccaaagacgcgaggtaacaacAACACACACCCTTTGTCC | |
| | both Guide 1 | ATGAACTCCAGCGCATTTGGAAGCCAGTAATG | |
| | and Guide 8 | CACCATAAGAAACGTGTCGAATAAACCAATTA | |
| | and the | GGGGTCTGGTGTCCGAGTCATGAGATAGAACA | |
| | region | GGTTCGAGGTTGTTATATATCAATCAATAATTA | |
| | between was | GAGAAGGAGCCGGTCTCTTGTGTTGAGTTGAC | |
| | deleted | TCGATGGAGAGCTTAGGAGTTAGAAAGGGTGC | |
| | | ATGGATCCAAGAAGAGGATGTTCTCCTGAGGA | |
| | | AATGCATTGAGAAATATGGAGAAGGAAAGTG | |
| | | GCATCTGGTTCCCCTCCGAGCAGGTAACATGA | |
| | | AAGAGAAAGGGATCAGTATTTATTTGTGTTTTT | |
| | | TTACTTCTGTTTTGCTTAAAGAGTTTCATTTTCT | |
| | | TGAGTTTGCAGGGTTGAATAGATGCCGAAAAA | |
| | | GCTGCAGGTTGAGATGGCTCAATTATTTGAAG | |
| | | CCGGATATCAAGAGAGGAGAGTTTGCATTAGA | |
| | | CGAGGTTGATCTCATGATTAGGCTTCACAATTT | |
| | | GTTGGGGAACAGGCAAGTCTATAATAACTCAA | |
| | | GTACTAGCTTGATAATGATATTATATTAGTTCT | |
| | | GAAGCTGTTCAGAACTTACAAAAGAGCTGTTC | |
| | | AGTTGATACTTTGTCTGATGTTGTGCGTGTATA | |
| | | GATGGTCCTTGATTGCGGGTAGGCTTCCAGGG | |
| | | AGGACTGCTAATGATGTCAAGAACTATTGGCA | |
| | | TAGTCACCACTTCAAAAAGGAGGTTCAGTTCC | |
| | | AGGAAGAAGGGAGAGATAAACCCCAAACACA | |
| | | TTCTAAAACCAAAGCTATAAAGCCTCACCCTC | |
| | | ACAAGTTCTCCAAAGCCTTGCCAAGGTTTGAA | |
| | | CTAAAAACTACAGCTGTGGATACTTTTGACAC | |
| | | ACAAGTCAGTACTTCCAGGAAGCCATCATCCA | |
| | | CTTCACCACAACCGAATGATGACATCATATGG | |
| | | TGGGAAAGCCTGTTAGCTGAGCATGCTCAAAT | |
| | | GGATCAAGAAACTGACTTTTCGGCTTCTGGAG | |
| | | AGATGCTTATCGCAAGCCTCAGGACAGAAGAA | |
| | | ACTGCAACACAGAAAAAGGGACCCATGGATG | |
| | | GTATGATTGAACAAATCCAGGGAGGTGAGGGT | |
| | | GATTTTCCATTTGATGTGGGCTTCTGGGATACA | |
| | | CCCAACACACAAGTAAATCACTTGATCTGATTT | |
| | | AGACCATTTCAATCTTAATGTTTAAGTTCAAAA | |
| | | GTTGTTCTTGCATTCATGCTCTACTCCCTAATC | |
| | | ATTTTATGTTCCAAAGTTGTTAATCTTAGAAAT | |
| | | AAAAACAAAAATAATAAATATATCAATATGTA | |
| | | TAATGGTGGATAGGGATGTGAGCAGTGGATGC | |
| | | AAACTCAAGCATTGAATGAATGGTTAAGATTA | |
| | | ATGAACCCAATCATTTGAAGTGACAAAAGAAA | |
| | | GACATTTTAAAAGATTCTTAACAATATGAAAA | |
| | | AGTTGTTTCATATTTCTATCGGTTTCCAACCAT | |
| | | TAATTAGACGGATGGACCATTGAAATATCTCA | |
| | | TGCATATTGAATTAATAAA | |
| Full | Hypothetical | AATACACACCGATTGAATAGTTTAGGTTTAGA | 41 |
| GRET1 | repair | AAATGATATATTTGGAATAACAATTTTTTTTAA | |
| removal 2 | outcome if | TTAAGAAAAAAAAGAAGGATAAAAATGATATT | |
| | editing with | ATAAAAGTTTCATTAAAGCCTATAATATTCCCA | |
| | both Guide 9 | CATAAATTTAAAAACGTCTTCTCCAAGAAGAT | |
| | and Guide 10 | ATCTTATTGCTGCCTCTATATTAAGCTTTTTTTT | |
| | and the | TTTTTTTTTTTCCCTTCATTAACATCAAAAGGTC | |
| | region | TTTGGAGAAAATGATGCAACGACCTGCAGAAT | |
| | between was | TAAGAAGTTAATGATGTGCAAAATCATAACTT | |
| | deleted | TCTTCTATTTTTATTAACAATAAAATTTTTTTCT | |
| | | TAACACTACTAATCCAAATATTCACTTCAAAAC | |
| | | TAATATTTGTTATTTTATTAATTTTTAAAAATAT | |
| | | AATAATTTAATATCTACACATTACTTATTTTAT | |
| | | CAAAATTTTCAAAGGTTCATGTGAAAATTTTAT | |
| | | ACTTTTATCAATTTCCATCATTTTTCAAATTTTA | |
| | | AAACAATTTTAATTTATATGCAAATTTCTACAG | |
| | | ATATTTCCATAAAACTCGAAAATCAGTGAGGG | |
| | | TAACAAAGTCAATTTTTTTAACATTGGGTATAT | |
| | | GTATATATTTGAGGGAATTGTCAGTGAGAAGA | |
| | | ATACATGGTAGGAAATGACACACGTCCCAAGC | |
| | | AACAGATGGATGGTTGAGAATGAACCCGGTCA | |
| | | TTGAATTGACATTAGAAAATGTTCTAAAATAT | |
| | | ATTTTTATCGACGTTACTGATCAGAAACAAAGT | |
| | | CCTCCGCGAGCCAGAGGCATATCCTAATGATT | |
| | | GTACCTTTCCTCACTGACAATTCCCATTAATTA | |
| | | GCTGCTGCCACTGCATAGCGGCTATAATATAA | |
| | | TGGTAGAGGACCCATGGAGCTTTCCTTTTCAGT | |
| | | GAACATGGGTTAGTCGACAAAAGAAAATGTTC | |
| | | AAGTTGAAAGAGGAGCGGTGGCCCTCAAAGGT | |
| | | TCCCATCACTTGGTTGCTTTTTGTCTAAGGAAA | |
| | | CAGTGGTATCAGAATCCAAATCTTCTACGTAAT | |
| | | GTCCCATTCATCCTACCAATGTCCATATGAATT | |
| | | CCTCTGGACGTTAAAAAATGGTTGCACGTGGT | |
| | | TGCCTTCAGGATCACACCAGTTTATACATTTGG | |
| | | ACCACAAAATAGAGATTGTTCATCAAGGATAC | |
| | | TAGTCAGCAATTAATTCCTAAATATCTCTTATG | |
| | | ACAAAGCCAGTAATGCACCATAAGAAACGTGT | |
| | | CGAATAAACCAATTAGGGGTCTGGTGTCCGAG | |
| | | TCATGAGATAGAACAGGTTCGAGGTTGTTATA | |
| | | TATCAATCAATAATTAGAGAAGGAGCCGGTCT | |
| | | CTTGTGTTGAGTTGACTCGATGGAGAGCTTAG | |
| | | GAGTTAGAAAGGGTGCATGGATCCAAGAAGA | |
| | | GGATGTTCTCCTGAGGAAATGCATTGAGAAAT | |
| | | ATGGAGAAGGAAAGTGGCATCTGGTTCCCCTC | |
| | | CGAGCAGGTAACATGAAAGAGAAAGGGATCA | |
| | | GTATTTATTTGTGTTTTTTTACTTCTGTTTTGCT | |
| | | TAAAGAGTTTCATTTTCTTGAGTTTGCAGGGTT | |
| | | GAATAGATGCCGAAAAAGCTGCAGGTTGAGAT | |
| | | GGCTCAATTATTTGAAGCCGGATATCAAGAGA | |
| | | GGAGAGTTTGCATTAGACGAGGTTGATCTCAT | |
| | | GATTAGGCTTCACAATTTGTTGGGGAACAGGC | |
| | | AAGTCTATAATAACTCAAGTACTAGCTTGATA | |
| | | ATGATATTATATTAGTTCTGAAGCTGTTCAGAA | |
| | | CTTACAAAAGAGCTGTTCAGTTGATACTTTGTC | |
| | | TGATGTTGTGCGTGTATAGATGGTCCTTGATTG | |
| | | CGGGTAGGCTTCCAGGGAGGACTGCTAATGAT | |
| | | GTCAAGAACTATTGGCATAGTCACCACTTCAA | |
| | | AAAGGAGGTTCAGTTCCAGGAAGAAGGGAGA | |
| | | GATAAACCCCAAACACATTCTAAAACCAAAGC | |
| | | TATAAAGCCTCACCCTCACAAGTTCTCCAAAG | |
| | | CCTTGCCAAGGTTTGAACTAAAAACTACAGCT | |
| | | GTGGATACTTTTGACACACAAGTCAGTACTTCC | |
| | | AGGAAGCCATCATCCACTTCACCACAACCGAA | |
| | | TGATGACATCATATGGTGGGAAAGCCTGTTAG | |
| | | CTGAGCATGCTCAAATGGATCAAGAAACTGAC | |
| | | TTTTCGGCTTCTGGAGAGATGCTTATCGCAAGC | |
| | | CTCAGGACAGAAGAAACTGCAACACAGAAAA | |
| | | AGGGACCCATGGATGGTATGATTGAACAAATC | |
| | | CAGGGAGGTGAGGGTGATTTTCCATTTGATGT | |
| | | GGGCTTCTGGGATACACCCAACACACAAGTAA | |
| | | ATCACTTGATCTGATTTAGACCATTTCAATCTT | |
| | | AATGTTTAAGTTCAAAAGTTGTTCTTGCATTCA | |
| | | TGCTCTACTCCCTAATCATTTTATGTTCCAAAG | |
| | | TTGTTAATCTTAGAAATAAAAACAAAAATAAT | |
| | | AAATATATCAATATGTATAATGGTGGATAGGG | |
| | | ATGTGAGCAGTGGATGCAAACTCAAGCATTGA | |
| | | ATGAATGGTTAAGATTAATGAACCCAATCATT | |
| | | TGAAGTGACAAAAGAAAGACATTTTAAAAGAT | |
| | | TCTTAACAATATGAAAAAGTTGTTTCATATTTC | |
| | | TATCGGTTTCCAACCATTAATTAGACGGATGG | |
| | | ACCATTGAAATATCTCATGCATATTGAATTAAT | |
| | | AAA | |

Guide RNAs and Cas enzyme, such as the Cas12a enzyme, are introduced to plant cells as described above. It is anticipated that these results will demonstrate that the methods of the present technology are useful for producing an edited genome in Vitis plant cells and are effective for producing a change in the color of a plantlet produced by a plant derived from the Vitis plant cells. In particular, it is expected that the foregoing method will be effective for producing a change in the color of a plantlet produced by a plant derived from the Vitis plant cells from green to red. In some embodiments, it is expected that the foregoing method will be effective for producing a change in the color of a plantlet produced by a plant derived from the Vitis plant cells from green to orange. In some embodiments, it is expected that the foregoing method will be effective for producing a change in the color of a plantlet produced by a plant derived from the Vitis plant cells from green to pink.

### Example 10. Validation of gRNA Efficacy

Applicant validates gRNA targeting and CRISPR-based editing efficacy in grapes using a berry infiltration transient expression system. Berry infiltration methods and transient gene expression systems have been used for such validation experiments, for example, as described in Zhang et al. Canadian Journal of Plant Science. 100(2): 175-184 (2020), and Fu et al. The Plant Journal,. 43: 299-308 (2005), both of which are incorporated herein by reference in their entireties.

Briefly, bacteria, such as agrobacterium, are transformed to include a plasmid encoding a sequence corresponding to one of the predicted sequences resulting from gRNA targeting and editing of Gret1, as shown in Table 6. Applicant clones these predicted edit sequences into a binary vector for use in Agrobacterium, to create ‘scarred’ promoters in front of MybA1. Expressing these ‘scarred’ promoters mimics the effect of gRNA targeting on gene expression outcomes.

Following transformation, the bacteria are introduced to Vitis berries by vacuum infiltration, such as described in Zhang et al., Knockdown of VvMYBA1 via virus-induced gene silencing decreases anthocyanin biosynthesis in grape berries, Canadian Journal of Plant Science. 100(2): 175-184 (2020) (https://doi.org/10.1139/cjps-2018-0322), or Fu et al., Virus-induced gene silencing in tomato fruit, The Plant Journal, 43: 299-308 (2005) (doi.org/10.1111/j.1365-313X.2005.02441.x). In some embodiments, the bacteria are introduced to the berries by syringe injection. Applicant then monitors the berry phenotype post-infiltration.

It is anticipated that the results of the foregoing experiments will demonstrate that the methods of the present technology are useful for producing an edited genome in Vitis plant cells and are effective for producing a change in the color of a berry produced by a plant derived from the Vitis plant cells. In particular, it is expected that the foregoing method will be effective for producing a change in the color of a berry produced by a plant derived from the Vitis plant cells from green to red. In some embodiments, it is expected that the foregoing method will be effective for producing a change in the color of a berry produced by a plant derived from the Vitis plant cells from green to light red. In some embodiments, it is expected that the foregoing method will be effective for producing a change in the color of a berry produced by a plant derived from the Vitis plant cells from green to dark red. In some embodiments, it is expected that the foregoing method will be effective for producing a change in the color of a berry produced by a plant derived from the Vitis plant cells from green to orange. In some embodiments, it is expected that the foregoing method will be effective for producing a change in the color of a berry produced by a plant derived from the Vitis plant cells from green to pink. In some embodiments, it is expected that the foregoing method will be effective for producing a change in the color of a berry produced by a plant derived from the Vitis plant cells from green to purple.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

What is claimed is:

1. A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least two guide RNAs (gRNA), or at least one polynucleotide encoding the at least two gRNAs, and
(b) an effector protein, or one or more polynucleotides encoding the effector protein;
wherein each of the at least two gRNAs hybridize to a Gret1 sequence, and each of the at least two gRNAs form a complex with the effector protein; and
wherein the effector protein is a Cas protein comprising a nuclease and/or an effector domain.

2. The method of claim 1, wherein the Cas protein is a Cas9 protein, a Cpf1 protein, or a Csm1 protein.

3. The method of claim 1, wherein the edited genome results in a change in the color of a plantlet produced by a plant derived from the plant cell from green to red.

4. The method of claim 1, wherein the edited genome results in a change in the color of a fruit produced by a plant derived from the plant cell from green to red.

5. The method of claim 1, wherein a plantlet produced by a plant derived from the plant cell comprising the edited genome is red, and wherein a plantlet produced by a plant derived from a plant cell that does not comprise the edited genome is green.

6. The method of claim 1, wherein a fruit produced by a plant derived from the plant cell comprising the edited genome is red, and wherein a fruit produced by a plant derived from a plant cell that does not comprise the edited genome is green.

7. A method for producing an edited genome in a plant cell of genus Vitis, the method comprising introducing into the cell a Clustered Regularly Interspaced Short Palindromic Repeats (CRISPR)-associated (CRISPR-Cas) system comprising:
(a) at least one guide RNA (gRNA), or at least one polynucleotide encoding the at least one gRNA, and
(b) an effector protein, or one or more polynucleotides encoding the effector protein;
wherein the at least one gRNA hybridizes to a Gret1 sequence, and the at least one gRNA forms a complex with the effector protein; and
wherein the effector protein is a Cas protein comprising a nuclease and/or an effector domain.

8. The method of claim 7, wherein the Cas protein is a Cas9 protein, a Cas12a protein, a Cpf1 protein, or a Csm1 protein.

9. The method of claim 8, wherein the Cas protein is a Cas12a protein.

10. The method of claim 7, wherein the edited genome results in a change in the color of a plantlet produced by a plant derived from the plant cell from green to red.

11. The method of claim 7, wherein the edited genome results in a change in the color of a fruit produced by a plant derived from the plant cell from green to red.

12. The method of claim 7, wherein a plantlet produced by a plant derived from the plant cell comprising the edited genome is red, and wherein a plantlet produced by a plant derived from a plant cell that does not comprise the edited genome is green.

13. The method of claim 7, wherein a fruit produced by a plant derived from the plant cell comprising the edited genome is red, and wherein a fruit produced by a plant derived from a plant cell that does not comprise the edited genome is green.

14. The method of claim 7, wherein the at least one gRNA comprises a nucleotide sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

15. The method of claim 7, wherein the at least one gRNA comprises SEQ ID NO: 26.

16. The method of claim 7, wherein the at least one gRNA comprises SEQ ID NO: 28.

17. The method of claim 7, wherein the at least one gRNA comprises SEQ ID NO: 32.

18. The method of claim 7, wherein the system comprises at least two gRNAs.

19. The method of claim 18, wherein the at least two gRNAs comprise SEQ ID NO: 26 and SEQ ID NO: 33.

20. The method of claim 18, wherein the at least two gRNAs comprise SEQ ID NO: 34 and SEQ ID NO: 35.

21. The method of claim 1, wherein the plant cell is a SUGRA35 cell.

22. The method of claim 1, wherein the Cas protein is optimized for expression in the Vitis cell.

23. A genetically engineered Vitis cell produced by the method of claim 1.

24. A genetically engineered Vitis plant comprising the cells produced by the method of claim 1.

25. A product comprising the genetically engineered plant of claim 24, or portions thereof, wherein the product has altered pigmentation relative to a product produced by a non-genetically engineered plant of the same strain.

26. The product of claim 25, wherein the product comprises a fruit.

27. The product of claim 25, wherein the product comprises a grape.

28. The product of claim 25, wherein the product is a wine.

29. A guide RNA (gRNA) comprising the nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO:
35.

30. A composition comprising:
(a) a polynucleotide comprising a sequence encoding a guide RNA (gRNA) having a nucleic acid sequence set forth in any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35; and
(b) a polynucleotide comprising a sequence encoding a Cas enzyme.


SEQUENCE LISTING (41 sequences)

SEQ ID NO: 1 - DNA, 25068 bp, Vitis vinifera
  source 1..25068: mol_type = genomic DNA
  misc_feature 2724..2942: note = Exonic region
  misc_feature 3029..3161: note = Exonic region
  misc_feature 3141..3159: note = Exemplary sequence targeted by gRNAs
  misc_feature 4182..4270: note = Exonic region
  misc_feature 10515..10572: note = Exonic region
  misc_feature 10932..11087: note = Exonic region
  misc_feature 12067..12215: note = Exonic region
  misc_feature 13333..13452: note = Exonic region
  misc_feature 13605..13818: note = Exonic region
  misc_feature 13798..13816: note = Exemplary sequence targeted by gRNAs
  misc_feature 13913..14015: note = Exonic region
  misc_feature 15740..15791: note = Exonic region
  misc_feature 20064..20113: note = Exonic region
  misc_feature 20649..20840: note = Exonic region
  misc_feature 23898..24064: note = Exonic region
  misc_feature 24653..24717: note = Exonic region
gaggtgcggaatattcgatgacggaagcaaggcaaggcaaggcaaggcaagccaacccactcaaaaaaccactatcatgtggaagtggcactcaaccgaaaccacatccataaaaccatctatcggtttcgcacaaacccaataccctagctcctctcttcttcaattaaagcgtaatctccttttctattcatacttcactcctgtccaaggtaacagttcctcatctttattcatcgctgttatctctacaatcgttttgctttgttaactaatgaaatgaaggttgagccgttgagcagcagttggttattattattattattattattcttttttgggtttgacgttggtgttaatttggattagagtttgccatatttgggaaatatgctttggaagagagctataattattttctgctacagggttgagtttgggttgccttagaccccttttgtctctgttttgtttgaatctcacaaggattgcagggacattgatttgattgttgagatgaagcttagaggtgaaaaatgttttgtatgagggggattgagagggatttttttattttttatttttgggttgcaatagtgattggttccgcgcttatggcttttataagtaggatatttttttatattttttttccctcaagagcaggagagccttcaaaaatgcttcaattatacctcatttctctctcgccctctctcacacacatactgcatattatctgcatggaatttctttgttgtaagaaaagaaaaagaaatgagaaaaacaacatttgttattatatgagcaaactctcaatttttaatgttaaagttgatattatgaaatttattatcataagatagctaataagactttattttgcagcatttacataagtgaaaaaacttatgagggaaatgaaatttattttcatagaattgcaaacaagagttgaattctcttttagtagtgggtgctaacaacttttttttgcagtatttacatgagtgaaaaaattaagtgggaaagataacttaatcatgaagcgctcactgggtatattctgtattggtgttcagttgggtctatttttgatacaaaaaaattctcttttttttttttgtatatgtttttaagtatgaactaaattggaggttaacaaacctatgaaatgggtataattatctatagttcgtgttttttcaacagttaatatggttatttaacatgctgacaatattctacaggggagataccgaaatatgagtgattccggtaatggattagatataattagcatagacagattgagacatggatataaaatttttatctttcaattacaagatggcatggtgcacacacaataatttttttgcgtaggccaaaaaaaaaaaaagcacaattagtctttcatatacagttgaagatcagcacttgttaggagaaaatgatgaagattcagagaaggagaaccaagagattgtcaattggctgcgccctctttagagatggatatgcatatttgatagcttcattaattgtgtgataacagaaagacatgatatttcacctaaaagaatacttaaaaaaaatattagatgctgaatggagaccctggaagaacttccccttctataatggcctagaggcatggaatggtgcattctctttacttcatgttatttctccattatttgtttttattagtttatccacatagtgctgttttctgtacttgcctatcttttaagaaaatttggtggatagagaaagcagtggatgagttgacacaatttgaattgaaatgatcatacattcttttttttttatttatttattttcttttatgatgatatggtaatacaatgggactaattttgaaaatttgatagtaggccaagggagggcaaaaggaactcttggactttgacagtcgtgtagatgatttaatattatttaaaagtcaaatgtaaattatttaaatcacaaaaatactagaaataaaaggattctctataacataaagtatttaatgaagctctaaaatggtttcttacaatgctgtcatacagtgacgtagtgataagatctgaattgactgtgtagtgcactctggtgagtaccctaactctattccagccttttttttcttcttttccttctagaaaaatttctgaaacgaaattcggaaagttgttgggattgttggaagagaagaacatgaatttacaaagttggaactctcggcaataaatttatgcaaccactcccaaactgtgagtccatgttttgagtgctggagcaactccttttttcatgaatgatgggaatatgcatagccatgcatccatttggagattgtgtcgataatgactatatactaatgttattgttgttattattattactattattatagagactctatagtgaagactgatactattattcttgttattgtttttactgccactattattattattattattattattgctattgttgctgtccgctgctgttatcatcattattattgatgttgttgctgctgctggtggctatatggaggttttgattctgtactattggaacatatgaactatctaatttgcattattctggtggctgatgtgcagaataactacagttggactgttgatttctgttcttaaaaatttgttccggtaaataattcagagattagatttggtgaaatgactcaattcagatatgtttctgcggtgaacttgagctgccaaagtaatataataaactttcagaactcccaatgtacctggagacatctttatattgattcagatcagaccaatacacttctttttggaggtggtgactctatgggtctcaagttgagaattccaaataagcattctattggaacaaggcggaggaaggatttctgccccttgcaggtctgtcttcacaaacttaagagggttttagttttccggactaactaaatcatcaatcagaaatgagttatttttttatgatacaggttgtttgcatggattatccaagaccagaacttgagaatactgtgaatttcttagaagctgcatacttatcctcatcctttcatacttctcctcgtcccagtaaaccattagaggttgtaattgctggtgcaggtggagaaatctaatcctatttgaatcttttatcttccatttgtttactggctattgtctttattttgatatctcttgctgtcagtttaggtaatcaaacttgtgtccatggaatttactgatattgttgtgccttttctgtggttggatataaatatcatgcatttgttcctcttaagtcaatctcaaaatgtgtatttgccacctcttttgttgaataaaggtatctgctggtatggagtgatgaattagggagctattgctacttgctgaatttgttagaatatgttaaaatcattatgtagactgggatgaggttgttactaccatatttatcactatagttggaagttttatgtatttgaaaacattccgtgttgctaagtaattatctgtaatgcttgcatcctggattctatttggtttaaatgtttaatttatgacgcggccgaatatcatctatgacttctgcattgcctttaaataagcgttcccaactttatagttggcatttccattggtcttatcatgtccttgaggcatggctcaagtggcaacggtttggggtggagatgtgggagatcctaggttcaagttgcagcatgcacaaaaaaagataacttataaaatgaaaaaagaaaagaaaaatttattggtcttatcctgcaagagtagcctttggcacacctgaatggatcattggaaagccagatttagttgcttttcccaaaggtgggttccctctgacttgcaaaagaaccttattgttattttttttcctttcctatcatttagggttttaacttgtgtctcttttaatcgtaattttggtcctttttgttcctacatttttcctctcgtattttcatccattgtggattgtaccttctttgatcatacttataaaactttagttaccataaaatgattatgtaatgcaaattgcttttgaagagtggtttgccctttgataccttctttggttgtgacatcaaattgccaatttttaaacacttgcaggtttggctggtttgtctactgcaaaatatttggcagatgcaggtcacaagcctatattgttggaagcaagagatgttttaggtggaaaggtttatgagatgctctatttttgcccctctttttaatttttagtcattaaacttctcttgcttattctgtcatataaaattcaaactttctccccttattggttattataagacataagaacagtgttcacctagaaggagaagtaacagtttatatctggttagtgtttttttttaattaattttttattcttactttatttttattttattttttaactttttgagggggaaacaaacagtttatttaatagagaaatggatgaaacagtacaagaagaggagctatcctaccaatagagataacataaaccaaaaagagcatagaagggcacaaaagagtacaggtgaaggtggtaaaaaatccctcacagaggacaatcagttataggctccttggtcaaatagaataattttttagttttccttaattgtaaggtttatatgccaaatatgaactttatttagttcttaattttgcttaagaaggcaaaagatgggcaaaattcaaggcatatgatcatctatatatatattttttgattggtaaaggcatagagagatagtatatataacgaataaagggcgtccaagagccaacccaaagcatatagggagtatataacaggcgcctaaaggcaatagcaaaaaagaaaagggaaaaacaaaaacttacctgccctcatctagaacccaaccaatcaaataaattgattaaagataaaggaccttcatctataaacaacttagtctaagaccaaaaattacaaacaaaagaatttttcatcctttgagtcgaaaacacttcgttatcaagggtaatctcgtttctttcttcaaaactgtccaaaagagacataagggagttgccatccaagcctttttatgctttttgcccacaaaggaaccatgccaaccaagaagggtctctctaaccaacattgggaggacccatgtcacaccaaaaagagcaaataataactccttcaaaaccctagtcttggtacaatggattagaatatgatcaatcgactctttttcaacacaataaaggaatcatctattagctagggacctcccctcctatttagttgatccaaagtcaaagtcttccctcacaaagcttcctaagcgaaaaagcccaccttagtaggaacacaagggctccaaatggtgctcctcaaaaacgagactgcagttctaagctccagtgcaccataaagggacttaacagaaaatttcccatccttggtctcttctttccaaagcaccctatcctccaagtcagtaatcaccctcttaccttgtaaagtcacgaggaaccacctctccaccatctccaccatctccacctccccatcattgaaaggcctagagaaccgaggattctaacccccttcttacctgaaatgtcccaaacctttgctactcacgccttttttgaaacctcttaaggcatacaaagaggggaaagaaacacacaaaatatcgtctacacaccacttatccttccaaaatctcactctttctaccatcgcccactgagaacacaattttgttattcaataaagacacttccttcttgatttccttccataaccccacgctatactcatccctcacttcacaagatgaggcttcctctccaaagcccctccctccccaatagaaaggccctttggatttgctttaatctcaatataaccactcttagcatacaaaacaaagacatgaggtaaatgggtatgctagacaacgtactccgaatgtgagtgattctccttcctttaaatataaattgccttatggctagcctcttacgaaacctctcctccaccccatcccaaactgccacagatttatgcagagcacctaagggaagtcctaagtaagaaaagggaagcgtccctgccttgcaatcaagctcaagggctaaaatctttgaattgtctaccctcctcatcagtaaaatttcattcttgtccaaattgattctcaaccttgaaatgactccaaaccacattaacaactagcttatgtaggtcatctgatcctaggaggctttacaaacaacaagcgtatcatcaatgaacaacaaatgagtgatacgagccccatctctacacttttcctttaccttacaacttgaaaaaatcccccactcattgctctattgataaggctgcttaaaacctccatcacaatcacaaataagtagggtgagagaggatctctttgccttaattctctagtgttgtggacaaaacttgaaggtgtgccattaaccaaaatagcgaacgtcatcgtagaaatacaccaccttatccaacttgcccacttctccctgaaacctattttttgcatcacaagcagtaagaaattccaatttatataatcatatgttttctttaagtccaacatgcacaacacgctattctcaatccttttcaatagagaatctatgacctcattagcaattaacgcaacatcaagaatttgtcttccctcaacaaaagcgttttggggcgaaggcactaccttgcccactgccttctttagcctattagctgatacttttgccaacaacttgtataggcccctaccacactaataggttggaagtctctaaggtcgtcaacccctcatttttttgggactaaaactaggaatgtagtgttcaagctcttaacaaatcatcatttctcatgaaattctctaaaaaaactcataacctcatccttcatgaactcccaattgaactaccagaatgccgcatagaaatcgttcaagccaagtgctttattcctgttcaactttggaaggattgaaaagaccttttacacagagaatgccttctctagcctgacaacatcctcccttccaattttttcaaaatccaaccaaatcaaactaggacaccaatcatcaagttttgttagcaagttctggaaagcgttgactacaccccccctgaatctcatgctcctctaatttccaagttctgttaattttgatcttagtccaacaattcttccttctgtgggagttagccattctacggaagaagccaattttcttatcacgttccctcaaccacagttttctagatttttgtctccaattaatttcctccatgagagtccatttctcaaacttgtcctttgcctccttcctaatttccacctcttccattgttaacactatcaatttctcctgttcatcccagaaagatgctttgtgtagcgccaaactcttattaaccccaatcttcccaaaaacatcattgttccagattctcaaattggtctttagagccttcaactttgctgccataacaaagctacaagaaccgctgaaattgaacccttgccaccaactcttcagtaactccttaaattcctcctcttttaaccacatattttgaaagcgaaaaggaattgggcctctcctcacccctcctccatctagcaaaatgggacagtgatcatacactggtctaggaaaagtgcactgcaccaccccactgaaatggaactcccaatcctccaaaaccaaaaaaagatctagttttgacatggattgactattcaacccacctctctaagtaaacaaacccccttgcaagggaaggtctctcaaattcaaatcatcaatcatctccgagaaccttctcatagctaaagacaccttcctttccttagtacgctcattagggaatctaattacgttaaaatcccctccaatgcaccagggatcattccataacccgcggatgacccctaactcttcttaaaaaaattctttgcttctcttcaaggtaggaccataaacacccgtaaaaatccacacaaatccatcctcatagtttttaaaatggcatgagattaagaattgacccatctccatcccaaccaactctagcactctgttgtcccataatactacgaccccaccagctacaccctttgcattcattgctccctactctagaaatctccccattcttagacttagcaccaccccaagggacatattctaaattttagtctcttgcagatgaaccaaatgcgccctttgagacctgattaatgccttgattaacttccacttgttactatcgttagcccctcttacattctatgataagatttttatcttcttctaaggcttgaaactctaaccccttctcctctgtccatcccactttctctgctcactccattgttattagtcgaacattataatttttttagttcatgttcaaacctagtagaagtttaacatcccctctatgatcatctatattattttttactaagttgaatatgaaggtcaaggagaaagatctaagtgaactcacaattaggaaaggaaaagtaggggctttgaatcatcttaaattcattttaggtgaggatgagagagttttagtgaaggaataagagatcaatggcaggaggaagagtggaaacaaaggacctttataggtgtattgaattcatggaggacatgcataatgattagtctgaaatggaccaccaaactcaaaccatcctgctttacttctattgttaatgcccttttcattattgcccttcttatggagtgatatttatgtattgtctagtagtttgtggggttagggatttgtcatgtcatcttcttaaattttagggctgcaataggttagataaatattgtacatttccaagttccagttatgcaaagactatttctttgagtctgtatgaaaggttggaagttgagaagttcagtgcttttgtaatcctagctcatactttataatgatttgatctccccgtcatccatagatgtagaatcactggcatatcacatcaaaatgggttttttttgtgcaattgcttttatttaattttatattttctggtatttgtgcttgactgacttggccttgcagcactactaaggtcaagattgttcaattatagtgaagatgactggtaccaagtctgagttggagaattggaaagattaacttatgatatggcaaggggcaggaaaaatctcttggtaaagcagggcttgcataaggcttcatttagaaaaaacaaagaactttcaataataatagaggaggcctgtcttaataatggataaggaatccctttctgtggaaggttcattacaatcaggccatcagccatgtttagatcttcctggagagcaaaaaacttatcattaatagccatcccagtaccagctggcaaggaaaagacctgcaacatctcccttacagcacaggaaggaggggacaatgggaaaataatttcctctcttttttttggttactcatgcaagcagctttccaatctagcaatataatgtttggggaatgccagggaactccttagtggttaatagccaaagaggggtgattagaaaacaacttatatcacacaaatttccaggtgtccagtcttatcctgaaaaatcctcactccaatcataatatcatagtcaaggatcagacaccaccaactctactattcttcctcttgccaaaatcccttaaaaatgatcagcatgttgttacaaatatcgttttaagcatgcccaagtgaatctaaccaaatataatagtctttgtcagatctcataagcaagaggacaatggaggtttgatggtcgaccagtcccctcactggccatatgcagaacacatgcattatgcctaatttctctatatgacctctgaatatgaagtaattcattgttttttctggatagaaaatggttatattttcagttggtgcaaacaaacctaaagaaaactctaggtaagaaaaggtcccttctgtctggagttcaaaaatgcttgtcaggaatagaagaatctttctccaagaagtggaagacagaggatgtgggtaatcggtttctctatcaatgagaatttggtggaacttgaagttccaagtatgagatgcagtaggcttgaggaaataccagaaaaggggtatctatcctccatctagaaatactttctaagaggagataatatgtacatagagaggtttccccactattgaattcatatggttgtagttaaaatctagttgtggaatagatacttttagttcatagttggaatataaacttattgcaatactaattgtataatagatttaacttcctacttaatttccccttttattgttggaagtttctttcatttagtgaaggctagtctctcttgatataggtggctgcatggaaagatgaggatggagactggtatgagacaggcctacatatattctgtaagcttatgtaactctcatttttttagtttgtgattgtgaaccttaattttgttggtgtcattgcccaggaagggcttctgatcggagttttggtttttaatcgtttcatatcataagtttcatttttttccctagtacatgccacctttcaggctatttgttcttttaatgttgtcatgcatggacctgtggaaattttgtatgaaacaactaattttgatgtcttttttatccctattttgtataaaatgtactgatccaccctatcctgatacatcatcatgtctgtaagaattttttacctaattcttaatctgattcttggtaaaataataatttgtttgctctccttttagttggggcttacccaaatgtgcagaacctgtttggagaacttggtattaatgatcggttgcagtggaaggaacattctatgatatttgcaaagccaagcaagccaggggaattcagccgatttgatttccctgaagtccttcctgcacccttaaatggtgagcaactcataagcatcattagcaatatttaagtcttgcatctttgtatatgatgagcacaatttaatttttcttttaatgaaaaattcagtgtgacagtggttggaaacaaatggtaaaattggttgttgaaattggatagaatgccacggcatcttgtattagtttcgatgagggagatcattaacttctggaaggcagtgcttggtctccttgtctatagtattaggcagggaagttaaaagtttagatgcagaaattattagctgttgcagttgtaagatcccatgcccatctgcttcagatatcactgatgccatcgatccttgttttttcttgattgagctatcaataaaaaattgctatagttcagttaaacctttgtcaactttatcaaccttaatagcgaaaatgtcccttcaaatataacataatatctccaaaacattgcagtacatctgtgcattcattagaatcttgtacataaaaaacaaagtttataacagtgtacatcattaggtgaaaattgttgtagttggaactttgtagcccaaatgcttccaaatttggatgagagttccaggatgagatggctatgctggctccagacctgacaacttatttggggcttcatataaacttaatccttgaaattcatggtcatttttttcctactaatgaaaacccatgtcaggatgcaaccatggcactccaatcattttttaatctgtccaattatcgatgtattatacattgtccattacaaccttttatggcttggatctcctagatgcagtcacttattggattgaattcctatcctttcacgtggaaacaaattgtctgtgggacatcctagacatatatttttttttttctttgtaattttctatgtttgtagattttttcctgtgtaaattatatattgctctccgcttgagttttaatgcatatttcttgtaaacatgattgacagggatatgggccatcctgaggaataatgaaatgctgacttggccggagaaaatcaagtttgctattggacttgtgccagcaatgctcggaggacaggcttatgttgaagcacaggatggtttaactgttaaagactggatgagaaaacaagtatggctgttgttgtgataatgctctattgcaggttcatgttaagttctgggtgaagctattgaacaatgaagctaagcttgagcagcttgtgaattttgttcttggaacatcatgagttgaaattgagcttagacaagtggcttgttggattacgtcataccccaagtctgattaattttgatgtttagcttaggtgaataacccaacatagctataaaatgtaaattggagagtaacttttcaccgacatatgtagaactagaaaatagagattatgacaatggtatgtgggatgcttcttcctttcaatattagtgtggaacgttcctagatgataataagcacacattttcgtcaccactttgaactctttgtattgacctttgttatcttgtgaaatctacctaagaatgtatgttacaagtttataaccatgtgaatttgttgattggttgggttgttggtaagggaggaagtttttttttggttttccccctgtggttgttccttggctttttttgttgttctcttggtgctctttgtatacttcctgtgtaccttgttgcactctttctttggtatgtgtttataatatctcatcattacttataaaaaaaaggcttataaccatgtccctggtgtctaaaactgggaaagttgccatgtctgacagatcattaatcatgttaatgcatgagagttgtgtcatgtgttcacaaatcataacatttctgctctatgagagaatccatgctgttagataaggctcatgccattatgagggtgatattagcatatgtgaagatgcaggccttgaatttggggaatatgagcagaagaatgtaagaacttggaaaccacaatgattaatgttttccacatgagatatttacatggttaaatctagtgtaaccacgtatggatttgttttgagggcgcaagcaatgttgtagtattccttatttgcttaaaatagtattggagtttacttgatcagtttgcataaatatagttgatttatgttgagctggattgaattctgtgtctattaagttaaggttaaatttcagaaaaatttaaaatcaccaatggttttcttctctctctttttcagggtattcctgatcgagtaacagatgaggttttcattgccatgtccaaggcactgaacttcataaatccggatgaactttcgatgcagtgtatattgattgctttgaaccgatttcttcaggtttgatcatctctattttcatagtgttctaatgaaagcttcatctctatttcatggatataaaactggtatcatgaccattgaaattgattttggagaagtttttgctacaaaatatctgattttactgtgtaagcgatatgcatcaacaggagaagaatggctccaagatggctttcttagatggtaatcctccagagagactctgcatgcccattgttgaccatattcagtcactaggtggtcaagtccaacttaattcacgaatacaaaagattgagctgaacaaagacggaactgtgaagagttttgtgctaaataatgggaatgtaattaaaggagatgcttatgtaattgcaactccaggtatgcattttaactctctctttgattgtacatttttcttcatgtgtataacctcagcttctctattgttgcttttgaaatttgttttaatcagttgatatcctgaagcttcttttgccgggagactggaaagagattccatacttcaggagattggataaattagttggagttccagtgatcaatgttcatatatggttagttctatagttttggatgttctctagcttatttaatgttaaaactatttgtgctagaaagtctaagaaatagaacataagaataaattaggaaatgggatgttggaatgagaggtgtactgccggcaacttttgccaccttaattgacattgagatcaagaattgagtgggtgtggaaagactttgtctggaagaaatagtgagtgggttcctattcacctggtttgttggaggggtttgggtggttatttgttatgattgtctttttttagagtgatctttcatgggctggtacttcctcctgatatgcaagcaagattactaacaggcaccaaaatggaacgggttctactatgtcaatttcgcttgcaaagcaacttgtgacagagccagtgaagattctatcttcttgttttggagcttgattccattcctgggcatcaggatgccaccgagtttgggggagggagggttgatctcaatgtgtttcatttcaataactagaagggttggggtttaggtaaaaaatgaggatgtttgaggagtttatggaaagtagaccttttttgtggttttccttgacaaacttatattagactagtttgaagggggtttgactcctaaaaatttacagaaggagagaaaggccttgaggtccacatacctctgcttaaggcctacatgtgaatggaataatggacttgtaggggtatggcgtacttaccctgaaaggaaactaacttttcctttaaattaaagctgtttatttctcttggaggtaaaatgggctaaatagctacttttactagaagtaaaagtggttacttaaccaagatgttactttcttatgtagaagtagtttagttaaggtggaagcaaaaaacctacttcactaggaaacagcttccattgtgaatgaagggagaggccttataatagaagatactggaataaaattaagttcttttcagttatggtgatatgcccaatactttggagtaattgcttcatccaaatccttcctagtaggtgttggtttctaatagaatcctaggtgctgattcctaggtttgacttgttacctatctagcattttttttgatctttcattctttcgaactgattttccttctattatttgtcagccattaaattgaaggactaaacctaccatccttattgtacaacctgttgtcaccaaacttacatccaaaggtaaagagaattctagcagtaggttagagaaatttctcctctctccttattaggagactctatatactttgtctggttgctctgcctagggctacttagatcatcttcacatcttaattttaatgtggtataactagtcaaagtaaatctcttttgaattaaagaatacatttgaagaccctagaaaatgatgcagatcaccagaaatctgcccaccctccttatatttagttcctgatccatcatcctccactctacacaaaccacttcccatctcacatattccccttagaatttgtagcacaaatagctaataaactgatatttcaatcaatatactgtcataaacaaaaaacgaaaaaaggggggagacattctaatccttgtcatggtctatctcagttgctttagtctttgggtcaatgcatattttaatatttgtcttagtcgttgatagttaatgtgtttgctcattggtcctgtaatgtaaattaattttgattgcaggtttgacaggaaactgaagaacacatacgatcatcttcttttcagcaggtcctgctcatcttttgaaataaattttatcccttaaagtcagcttgggcatgtagaagaagtatattaatgcttttgtttacaaaatctgctcatctcatgatcgatgctttagttaacagaatctgtatgctgatgacacgctgattttctgtgatgctttcgaggaccaattggctcaccttagctgggtgttgttgtggtttgaagtcatttctggtttaagaattaatctggataaaagtgagataattccagtggggagggtggagaatctagaggtgctggcatttgagcttgggtgtaaggtggggaagctcccatccttatatttagggttgcctttgggtgcacctcacaaatcagtggcggtgtgggatggggtggaggagaggttacggaggagattagcattttggaagaggcaatatatatctaaaggagggcgactcactctcattcgaagcacctcgtctaacgtgcctatttactttatgtcaattttgcgtatgcctagatcggttagattgagacttgagcaatttcaaagggaattcctttggggtgggggagctttggagaggaagattcatcttattaaatggccggttgtttgctcggataaaagtaaagagggtttgagggtaaaatgcctctctactcttaatagagttcttttgggtaaatggagttgacgctttatggtagaaaaggaggctctttggaatcaagttattagtaggaagtatggagtagaagaaggggggtggtatacgtgggaaggtagagagggttttggtgtggggttgtggaaggagattagaaaggaaggttctcggttgagcaactacattgggttttccatgggtaatggaagaagcactagattttggttggactcttggtgtggtgatgaggctttgtgtaactctttcccctctttgtttgctttggcggtttttaaaaaggagtgggtggcggaggtgtgggactcttcgattgagggggggagttggagtcctcattttttgagggctttcaatgattgggaggtagtcttggtggagtgtttgctcatgttcaaggaaagaaagttagtgcaaaagtggaggatagggtgatttggaaaaagacaaaaagtgagtttttcatagttaagtctctttatagtgttgaggagccaggaagtatagtccggtttccaagaaaaataatttggagcccttatgttcctctaagagtgggtttttttgcttgggaagcctcatgggggaaagttttaactttggatcaactcaaaagaaggggttggtcccttgccaatagatgtttcttttgtctagccgaagaggagtctactaattacatcttaattcactgcatgaagactagagttttgtgggagttattattcgctctctttggggtgacttgggttctcctttgttcagtaagagagactctccttggttggtttgggtcatttgtgggcaagaagcgcaaaaaagcttggaagctggcttctttatgtcttttttgggcagtgtggaaagaaagaaataggatagtgtttgacaatgaagagtttttagtgcataggttgaagtattcttttgtgtgtagtttttggtctttagacagagttgtatatagatgcttccctttatctttaattgacttttttgattggttgggttctaggtgagggcgagtgaagtttttgttttccctcctgttttgctcttttttgccttttggcacccaccgtatacttcctgtatgccttgggttgttttccgggcttcctttatctaattaatacactctctattgtgtttacccataaaaaaaaaattagttgttagtcttgtgagtgcttttctgcatatcttgcaaatcatggaatcactgaaatttacaacttattaaaatcatagaatggggggaagctgttatcattgtataaatgtaggacaactaataaagagttaacaggaccaactttttgttaaaactatctcttttctctaaagcttaagttattagaaaatggaataatatcaaactaacaacctctctcatccaaccccaacctgcatgcagagaggcaaaaaagatgtttttcaactagactaataacaaatgagggaataagtagatagtgaggcttgaacacaggacatgcaagacatgtttcaaaataacaaatggaggaacaaataaagaggctttctcaagaccttcaataatcaaagctttgatgttatgttgagctaccacttgcccctaagaagttgttgggcaagttgtagttttaacactttgagggatctctaccaaagggaaacagttttatcattttattttattttattttttgataagtaaaagaaattcattaaaagccaaagaggctaaagaaagggtatacatggagtataccaggatacaaaggtgcaaaaggcaaaaggacaaacaagcctgacccttacttggtagctaaccagtctacaaaatctatcaaagagaaagtgtgttcctctatatacaccctagcccaattcacaaaagtgtacaaaaaagtggatttaatttcttggtcattcttctcaacatcatcgaaggccctcctattcctttctttccaaatggtccacatcaagcagagaggggcaactctcaaagctttctcccttttcttgcccacaaaggaaccatgccatcccaggagggtccttctcacagagtagtgcatcacccattgcaccccaaaaagggagaagatcagaagccataacattctggccttctcacaaaacaaaagaatatgatctgtggtttcctcctcatttttacacaaatagcacttgttggggatgcaccatccaaatctctttaggcggccgatggtgagcagcctgctccaagccgcctcccaaccaaagaagctagccttaattggaactcaagacgtctaaatagttctagcaaagaagggaagtctaatgccccttgagagtgagctataaaaagacttgactgaaaaggtcctattcttgttctctttccatcgaagcaagtcatccacacctcttctaatagtcaaaggatgaagcttgtttaacaagatttctacttcccccacctcccaatcatttaggtgtctattaaaacgaagcccccaactacccccaacttcgtcttcctcccaagcctcagcaacccagccttttttgttgacggatagattaaacaagatggggaaagcatcttccagagattgattttcacaccacaagtccttccaaaacttcactctagtgcgattccctacgatgaagcgggagtgagttcgaaagttttcctaaccatttctgatggccttccaaaccactaccccataacggtctcttacacctttggagcaccatcccccctcttgaaggtcgtactttctagagataatttacttccatagggactcattctcattagcaaatctccacaaccacttcccaagaagggccttgttaaaaatagctaaattgcaaatgcctaggcctccatcctttttagcggcacaaataaccttccaactcaccaagtgaggtctattctctaaggcgcctccgccccataagaaatccctttggatcttttcaagccttgcacacactctcttggtaatcacaaagagaaaaagaaagtaggttgggaggctagagagggtgctttttaacaaggtaagctgaccacctttggagaggtattgccttttccaaagagacaaccttttcctgaatctttctttcactacatcccacaccctagtggatttgtaaggggctccaagggggagacccaggtaggaggtaggtaaactcccaatcttacatcccaagactaaggcaagagcctccataggaatgtcttcccccactggtatggcctcagttttgctcagattaacttttaatcctaaaatcgcctcaaaccacatgaatgtccaattttatttttaatgtcctattttgcatagaattttctattgagtaacaattttagtttttaagattacccagtgaagtgaaatatggctaacaaggtcttctagaccatttggtgagttccgagtttcttaatcttcaaattttggcccaagattgaaggggagagtttcttttgcttccaaatcatatttctttagtccagtatttgtcattgttgctgatttttcttactcagatttgatgttttgttttttccccccatgcagaagtccccttctgagtgtgtatgctgacatgtccgtaacatgtaaggtaaattctgttcataaaagcattgattctttcttgtatttatgcactttaagcctccataatgccatcattttagtcattttcatagacctcttagatgtcatttgtgacaaaaatgtctaatagaaccctacatgcccatttaccttcatgtgtaggcctaacacactggagaaacttgaaataatgaccatattgtatgctactgatttaggctcacttgctattacccatgacaaaaggaaaataacatgtttggaatttgctaaggccattgttagccttttgctttgtgttttgtgggtacttaggcatttgtgactaatatatcatcacatttgggaattataagggcatggatattttcaacttatcacaatatccacaacttgatccattgacaccaattctatattgttgtctgtataagtgccctcgagaatgggctaatatttttttcttctgtttttgttatatagacatatccagtatatttaaggatgaagctgttagttgtttcatgattcattgttgtgcaggaatattacaacccaaatcaatctatgctggagttggtttttgcacctgctgaagaatgggtctcacgcagtgactcagaaatcattgaagctacaatgaaggaacttgccaaactctttcctgatgaaatttcagaagatcagagcaaagcgaaagttttgaaataccatgttgttaaaacaccaaggttagaatgaaaaaagtcatttatttatcatctttccttttcttttttaattaaaaaatgccaatccattgaatagtaaaaaaaaatgtacaaagaaggatgagatatccttcataaaataaaggatctgaccaatagaaaacagaagggactacatccagaaatccaaagggaagaaaaaaggagagccatcaaaagcaaaacaagtgtaaatacaacgttatgtggatactcatggtggtaagaaatcccctgtaaagaaatctaactacaaagcccctcattttaccaattgatctgctagctgctttgctaaacaaggaatctaagggataaaaacaccctaaaactttagcaaatcagtgatttgacatgaccaaatatcaagtttacatggatcctagattctccttagcgaacatggaattgaatagtagaatctcctcccaaaaaaattgaacaaacccaaagaatttgccaacaaagggcatttcaaaaggtttgaatctctccctcaatagaatcttgatctagtgcatttagagaaagctctcaacacatttccacaatgatccatctgacccttcatcccttaattgtcctgggtttcctagaaagggtctggatctctacctctatagctattcctgatctagtaggcttatagaaagctctagtgcaatactacaatgatccctaatcatgcctacaatccctgattgtcctgggtttcctaaagagcaaccatcaaaattcagcttgggagtttctggtggaggtcttcccttggctcgaacccctacaactgccctcctagcattacaagctagactccagtcaagaaagatcaagcttaagggtacgcatgaaaatgcaactgtaatagatgctcacaaagaggatagaaagtgtactgaatcctgtgtggccttcaaagttgtccatctgtctttccacttgagcattacaagcaagaactccagtccagaaagatgaagctcaagggtgtgcatgaaaatgcaaatgtaatagatcctttgtggccttcaaagttgtccatctatctttgctactcgccacttaagaatttccaccaaaacctcaaaaggcaatattcatccacacttttttttgttggggtgagggggggaaggggagagatgattcaccgattttccataatcacaggcacacaacacatggttcagggctaagatcttactgggtctcctcaactgcaacatgtcattagtgtgacccctttagttgctactagccaagcaaaaagtattgaattttggtgggactttggcttaaccagaatgaaggtaattggatgctctgcagtagaaagggcttcacaagggaacaaactgagcatgaactctgaaagtcatggaccaacttatcaagaaacggagatgctaaatacacacatggtaaaggtgacaaaagtacaaagtgccttgattcttgatcatacaagtaatgatagattaaaattccaaaactttgaggaagaagaatggtctaggatagaaacaatatgcgaggacctatagatcaaatagaggttggagaattgagatcaaagagattggtctccctattagatatcttcccataactgaaatttttatcctttgccaaattggtagcaaatatatcgagaaaaattatggagatcatgggaaatcaaggacaaaggtatggccatttaattacgatgttgtcatcccatccactagactgtgtgccagagatactcttgataacctttttccatagtgccaactccctacgtacctccacaaccacattaccaagcaagcttcttcccttgaataaatcttcctaaaacccaagtcccttttatatttggcctacgcaactgatcctagctaatttgatgatctctccttcctacccccattccctgccacaagaagtctttatgaattctttcaatcctagatgctacttttactataatcttaaacactggtagaaacttagtgggtatatggaccaaataagactggactacagtggttctaccttgtagagataggtaggctcttcttcaaccttcaaatttatgatatatgctttctaaaactagatcccaggaaaaggaggctctagggttctgtcagagaagaccaagtattaaagagaccaattagagttcctacattctagaatggtagtttgaccatgaatttggtcatcacctaaaatgataccttacaaactactcttgttgatgtttatcttcagacctcatatgttgaataagctataacattaatcacctaagctccagcactgaagaaaacaatagtatcatctgcaaacttgtaatgaggtgttctaaattgacttctatccgccaaacattcttccagaatcactcctttgaatggagtgaagaaaatcatgaattaatctgttagcctaaatatgtcctatgtgtatcttagggttgccatcctagacatggctgaaattttttttttttttgtcttgatttatgacattattctacttcacaggtcttagcttcacatgcgaagtatttatttgttcatttggatgctggtgagcatttcctagttagctgcctacttatgtactggttgcttcaaaactagtattgtataacatatatgtttgcttaaaagttgcccccctgggtttagctcagtagtagcgccctttggcaatgaactagaggtcctgagtttgagcctctctggtatttaagtagtataggtagaggggcggaccagtatttacctggtttgcttcatcagcagttggaataagtgtcttactgttgggggattccacattatatacacacacactcaaatgtcaactgtttcaatattcactaaatgtttttttttgctttttcactttataattattacttagtagtttcaatatttattatatgttttttttttccacgtcaagttattgccttttgattttcgtttatttagtactattatagcatattgtaataattttaagctattggattcaacttcagatctgtttacaaaactgtcccaaattgtgaaccttgccgtcccttacaaagatctcctatagagggcttttatttagctggggactacacaaaacaaaaatacttagcttcaatggaaggtgctgttctgtcagggaagctttgtgcacaggctattgtaaaggtaacttcccacaggaagactccaatatttgcatcaaatttgtactggttaaaaaaagtcatacttctctgatgagagtatacttttattattttcaaactttgttattctgcttatagatataattgcattctcttagaaaaggttgagtgtaggttttttcgtgtgtgttttggctgctaggtttttgactccaagatgcctttggcattaactcagtaaggttatcttttgatgatatacataagcaaacataatgttgtaattgttaatacagagatcctttgtggattaactagtgaaatgactggttgctataacatggtaccacactgcataacatatgcaatcaccataacgtcgttgtttctatggtaaagatcctttgtggattagcatggggaaataatgggttactctaagatggttcttcattggactatattgcgtaacctgaagcaataggcttcagttgcaatacatatccttaaaaaatatatggaagaaattacggtctgataagggctctgtttgttgacagtgcttcatgtgttttatgcattactcaatccctttattttgcaacatacaggactatgaattgcttgttgctcagggagaacaaaagttggccgaggtcagcattctcagttaaaccacttgaattggaggaagtttttgcaacagtacctgggaattacccaactagcgcttgcagatcaatggcattttacaagttattaaccattcaccagtaaaaattggtatttatgctgtggcaatttcctagctctttgaaagtaacttctctataaaagaaagcatttaaactgatttgatttattcatgttataaaagcatcctttactttttcaacatttttgtggtcttcccataaaacatacatagaacatacctaattgtaaaaacaatccaactgagaagatggcaaagaaactctatttgtctgtatattttgttgacctaatgatacaatttgggagaa

SEQ ID NO: 2 - DNA, 22 bp, synthetic construct
  source 1..22: mol_type = other DNA
aggttgtaattgctggtgcagg

SEQ ID NO: 3 - DNA, 22 bp, synthetic construct
  source 1..22: mol_type = other DNA
cttatgtaattgcaactccagg

SEQ ID NO: 4 - DNA, 60 bp, synthetic construct
  source 1..60: mol_type = other DNA
ctagagtcgaagtagtgattgaggttgtaattgctggtgcgttttagagctagaaatagc

SEQ ID NO: 5 - DNA, 60 bp, synthetic construct
  source 1..60: mol_type = other DNA
tgctatttctagctctaaaacggagttgcaattacataagcaatctcttagtcgactcta

SEQ ID NO: 6 - DNA, 60 bp, synthetic construct
  source 1..60: mol_type = other DNA
ctagagtcgaagtagtgattggatgcttatcgcaagcctcgttttagagctagaaatagc

SEQ ID NO: 7 - DNA, 60 bp, synthetic construct
  source 1..60: mol_type = other DNA
tgctatttctagctctaaaacctccgagcaggtaacatgacaatctcttagtcgactcta

SEQ ID NO: 8 - DNA, 22 bp, synthetic construct
  source 1..22: mol_type = other DNA
tgtcccaggattagaattaggc

SEQ ID NO: 9 - DNA, 25 bp, synthetic construct
  source 1..25: mol_type = other DNA
agccctcttctttcgatccatcaac

SEQ ID NO: 10 - DNA, 46 bp, synthetic construct
  source 1..46: mol_type = other DNA
cctgtcaaacactgatagtttcgacttgccttccgcacaatacatc

SEQ ID NO: 11 - DNA, 53 bp, synthetic construct
  source 1..53: mol_type = other DNA
caagcttcacttcactagtttgcttttattcactagtataaccatggtattgg

SEQ ID NO: 12 - DNA, 24 bp, synthetic construct
  source 1..24: mol_type = other DNA
ttggaggtggtgactctatgggtc

SEQ ID NO: 13 - DNA, 23 bp, synthetic construct
  source 1..23: mol_type = other DNA
cacccaaacccctccaacaaacc

SEQ ID NO: 14 - DNA, 25 bp, synthetic construct
  source 1..25: mol_type = other DNA
ccctaattcatcactccataccagc

SEQ ID NO: 15 - DNA, 26 bp, synthetic construct
  source 1..26: mol_type = other DNA
atggagagcttaggagttagaaaggg

SEQ ID NO: 16 - DNA, 30 bp, synthetic construct
  source 1..30: mol_type = other DNA
tcagatcaagtgatttacttgtgtgttggg

SEQ ID NO: 17 - DNA, 1291 bp, synthetic construct
  source 1..1291: mol_type = other DNA
  misc_feature 432..450: note = gRNA sequence position
  misc_feature 1005..1023: note = gRNA sequence position
aagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnngttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatattaatccaaactactgcagcctgacagacaaatgaggatgcaaacaattttaaagtttatctaacgctagctgttttgtttcttctctctggtgcaccaacgacggcgttttctcaatcataaagaggcttgttttacttaaggccaataatgttgatggatcgaaagaagagggcttttaataaacgagcccgtttaagctgtaaacgatgtcaaaaacatcccacatcgttcagttgaaaatagaagctctgtttatatattggtagagtcgactaagagattgnnnnnnnnnnnnnnnnnnngttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaata

SEQ ID NO: 18 - DNA, 959 bp, Vitis vinifera
  source 1..959: mol_type = genomic DNA
  misc_feature 1..115: note = Exonic region
  misc_feature 106..124: note = Exemplary sequence targeted by gRNAs
  misc_feature 203..332: note = Exonic region
  misc_feature 452..959: note = Exonic region
  misc_feature 803..821: note = Exemplary sequence targeted by gRNAs
atggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctga

SEQ ID NO: 19 - DNA, 22 bp, synthetic construct
  source 1..22: mol_type = other DNA
gatgcttatcgcaagcctcagg

SEQ ID NO: 20 - DNA, 22 bp, synthetic construct
  source 1..22: mol_type = other DNA
tcatgttacctgctcggagggg

SEQ ID NO: 21 - protein, 9 aa, unidentified
LAGLIDADG

SEQ ID NO: 22 - DNA, 120 bp, synthetic construct
  source 1..120: mol_type = other DNA
aaaagtcccacatcgcttagataagaaaacgaagctgagtttatatacagctagagtcgaagtagtgattgnnnnnnnnnnnnnnnnnnngttttagagctagaaatagcaagttaaaat

SEQ ID NO: 23 - DNA, 120 bp, synthetic construct
  source 1..120: mol_type = other DNA
aaaagtcccacatcgcttagataagaaaacgaagctgagtttatatacagctagagtcgaagtagtgattgaggttgtaattgctggtgcgttttagagctagaaatagcaagttaaaat

SEQ ID NO: 24 - DNA, 119 bp, synthetic construct
  source 1..119: mol_type = other DNA
atagaagctctgtttatatattggtagagtcgactaagagattgnnnnnnnnnnnnnnnnnngttttagagctagaaatagcaagttaaaataaggctagtccgttatcaacttgaaaa

SEQ ID NO: 25 - DNA, 119 bp, synthetic construct
  source 1..119: mol_type = other DNA
atagaagctctgtttatatattggtagagtcgactaagagattgcttatgtaattgcaatccgttttagagctagaaatagcaagttaaaataaagctagtccgttatcaacttgaaaa

SEQ ID NO: 26 - DNA, 23 bp, synthetic construct
  source 1..23: mol_type = other DNA
ctcttccttggacagaagaagct

SEQ ID NO: 27 - DNA, 23 bp, synthetic construct
  source 1..23: mol_type = other DNA
atcgtccggatcatcaattaaag

SEQ ID NO: 28 - DNA, 23 bp, synthetic construct
  source 1..23: mol_type = other DNA
ccacgatatctagacagcagcat

SEQ ID NO: 29 - DNA, 23 bp, synthetic construct
  source 1..23: mol_type = other DNA
tcatgatgacattcggaggcagc

SEQ ID NO: 30 - DNA, 23 bp, synthetic construct
  source 1..23: mol_type = other DNA
ccaggtattaagaagttaccagg

SEQ ID NO: 31 - DNA, 23 bp, synthetic construct
  source 1..23: mol_type = other DNA
atccagtcgtcattcctgcaaga

SEQ ID NO: 32 - DNA, 23 bp, synthetic construct
  source 1..23: mol_type = other DNA
tgagttgagatcacgcgtccatc

SEQ ID NO: 33 - DNA, 23 bp, synthetic construct
  source 1..23: mol_type = other DNA
gcggtccacgtagtaaccagatg

SEQ ID NO: 34 - DNA, 23 bp, synthetic construct
  source 1..23: mol_type = other DNA
ccagagggaggggtccctacatg

SEQ ID NO: 35 - DNA, 23 bp, synthetic construct
  source 1..23: mol_type = other DNA
tccatgaactccagcgcatttgg

SEQ ID NO: 36 - DNA, 15573 bp, Vitis vinifera
  source 1..15573: mol_type = genomic DNA
  misc_difference 1650: note = a, c, t, g, unknown or other
taaattaaaacatgagatttttgaaggattagactagatagatagttgaatataggataataattttaattgatgaacataaaatttagacgaatatttgaagaagtaaattaagacatgtgatttttttgatggattagactttgaataactgataaatatgagacaaggattcgatttatgaaaataagatttaaacaaatatttgaagaattaaattaaaacaagggatttttgaaggattgacataagacccttcatccacacgaactaggcctggacgccaactcaagcccaagtccaatgtcttcaggggcaaacccaatacaaccatccaacataggtaagcccaaaacccaatggcttcaagggcaagcccaaaacaagcatccccatcacaagcttgggcctagttcctcaacccaagcccatgttcaatgctaatatgggcgggctcaaagacccatcttccaacagtattttaatttgaaaaacccaaaactacaagcttgaaactggaaaactcataagatacctatcaaaacatggaagtggagccaaagatgagggggaggaggcggagctacgggaaggatgaagggaggaggagctccaatgccgccattgattcagctgactgagcgagcttgaggaaatgggtatgaaaggtggcggccatgcatggctatgcatggccatgcgggggtgaggggaatgggcgtaatgggcatgggtgggaactaagatagctggaacatggtatgggttcaatgtggccatgcccggccatgcaagggtggagagaactggaagttctcaatgccgtaatcaaacatcaaacccaggtgggaatccaacctcttgcaggggaaaaatcaacaaaattatagaaagcaaaaaaaacagagcaagcacatgtaggcaatcgtatttgagtaaaacaaatttgtggttttggatacccacagaaaatcacaaaataatcttcgtagactaacagaggaccttacctgaatactccacaacgccaccctcctctctctcgtctgaatatccccacctcctctcccaaacgtccttctccttctgaaaagatcaatgtttcagaaaaactctctcaaaggctccctctctaaggtgtcttcctctctggttccttttccttcccatctccagcactcctgcccctcgagtgtatccttcctgttttttatttcttccttggcaagccattaccaaccttactattccttcattgctggctgatcaaccaccagcatggcccctgccatctccatgcagccggtttcccgggagaaaaggccccccccccccccccggcccccctttttttaaaaaaggaaagccaagaaaaaaaaaaaactctcttccgggcccccccccaaagggggctaccaatacccccccatttaaaaatttaagtttaaaaaatgaaataatttgaataacaatttttttttatttaaaaaaaaaaaaaaggaaaaaatgaaatttttaaaattttctttaagcccaatatatttcccactaaagtttaaaaactcttctcccagaaaaaatcctattgctgcccctatattaaaccttttttttttttttttttcccctccttaaactgcaaaaggcttnctctacagtaatttggggcaagttatgagagttgtgattctgagccctgagaaaatggaaatttcatacccatggtttttttgggttttttttttggaataaaaaaatttggggtttgggatacccccaaaaaatcaaaaaatattttttgtaaaataaaaggggcccttccctgaattttccaaaacccccccctcttttttttttttgaaatttccccccctcttttcccaaaagtctttttcttttgaaaaaaataaatttttcaaaaaaattttttaaaaggccccctttttaagggtttttccttttgggttctttttccttccctttcccaccacccctccccctggggggttccctccctttttttttttttttcctgggaaaccctttcccacccttatttttccttctttggggggggataaacccccaccagggccccccccttttccaggcaccggttttcccggggaaaaaggccccccccccccccccgtccccccttatttcaaaaaggaaatgcaaagaagagagaaaaactctcttcggggccctccacaaaaggggtctacaaatacacaccgattgaatagtttaggtttagaaaatgatatatttggaataacaattttttttaattaagaaaaaaaagaaggataaaaatgatattataaaagtttcattaaagcctataatattcccacataaatttaaaaacgtcttctccaagaagatatcttattgctgcctctatattaagctttttttttttttttttttcccttcattaacatcaaaaggtctttggagaaaatgatgcaacgacctgcagaattaagaagttaatgatgtgcaaaatcataactttcttctatttttattaacaataaaatttttttcttaacactactaatccaaatattcacttcaaaactaatatttgttattttattaatttttaaaaatataataatttaatatctacacattacttattttatcaaaattttcaaaggttcatgtgaaaattttatacttttatcaatttccatcatttttcaaattttaaaacaattttaatttatatgcaaatttctacagatatttccataaaactcgaaaatcagtgagggtaacaaagtcaatttttttaacattgggtatatgtatatatttgagggaattgtcagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgcgagccagaggcatatcctaatgattgtacctttcctcactgacaattcccattaattagctgctgccactgcatagcggctataatataatggtagaggacccatggagctttccttttcagtgaacatgggttagtcgacaaaagaaaatgttcaagttgaaagaggagcggtggccctcaaaggttcccatcacttggttgctttttgtctaaggaaacagtggtatcagaatccaaatcttctacgtaatgtcccattcatcctaccaatgtccatatgaattcctctggacgttaaaaaatggttgcacgtggttgccttcaggatcacaccagtttatacatttggaccacaaaatagagattgttcatcaaggatactagtcagcaattaattcctaaatatctcttatgacacatgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttcagcttcttctgtccaaggaagagcaaacgacgctgacagagcatagacatccggacaaccttcataatgtatccgctccactatacaatccggatagtcagcatgtgaccatccggatttaatcgtccggatcatcaattaaagtaaagcaagtcttacacgctatcacgacaaccagccatggcccacgtcccatcatctgcagagtgaaaggacgggtcgaggtgacaacaagtcacttcccacgatcattctacatgatcatttcccacgatatctagacagcagcatcacctaccacggtttctgacagccgccagtagggtggcgatgaccatgctgcctccgaatgtcatcatgacaaacataaaatatctcctcgccattaatgagaggaacagtacccctgaagctgtatatatatgccttcgcacgaagaagaaggggatcctcctggtaacttcttaatacctggtaaaaggccaactgatttatattcctctctctgaccatggctaacaaaaccatcggaggatgcgtccggacaccctgtccggatgccttcttgcaggaatgacgactggatcaaaaacctttatgagttgagatcacgcgtccatccatctggttactacgtggaccgccaaagacgcgaggtaacaacattggcgctgtctgtgggaacttttctttactttgattcagtcactaaaaacaatggccacaccatcccaaagtcattcatctggtaggggagaggatgataattttgaatggcgcctagccatcgaaaggagacagttggcaagcgaaagacagctaaaagctctcctccaggagacagaaagattgagagaagaaaacgttgtgttacgcatccaggcttcaacatcagagcctcctcgacttcaacattcgaggggccaagtagcaaactcaaggcctcagcaagaaccagagtcaatataccctgggacaacaggggcaatcccaggggcatgcaacgtgaggcctcatgagccatgcacgcctatgccttgagctccccgtgaggaaagctcagactctactcacttttcagcaaaaaggcaacgtgataaaaagtcgcggttgtcaaattcgatgcgcgcaagactaggcccgcaagaacctagaagatcaaggccacctatggccacaacctgggcaccacaccctgaccccatggcaacccccatggtgcaaaacgatcacccgcatcgtgaccccatagtcacccctgtgatgcggaacgttcatctgcacctagcggaatagccaactgggagaaacatcccaaacgggccacccgttggctccatcagcaaaaggctggatgacatgctctccacgcctttctgctctcatatcattaattatgagcccccaaggggattcctcgtacctaaattttccacatacgatggaaccagcgatcccttcgatcacatcatgcattatcgacagctcatgacgctcgatattggcaacgatgcgctgctatgcaaagtatttcccgccagtctacaaggacaggccctctcatggtttcatcgcctacctcccaactctgttggcaatttcagggacctgtccgaagctttcgtgggacaatacctgtgttccgcccgacacaagcaaaatatcagcaccttgcagaacataaaaatgcaagataacgaatccttaagggaatttgtgaagcggtttggccaagccgtacttcaagtggaagcttgcagtatggatgctgttctacagatcttcaaacgaagcatctgtccaggcactccatttttcgaatcactggctaaaaagcctcctactacgatggacgacttattcagacgagccaacaaatattcaatgctcgaagatgacgtacgtgcagccacccagcaagttttggttgccggacggccatccggaagtaatacggagagaaataccaaacctccggatcggccaaagccgtctgaccgaaggcaggaagggccaagtcgcccgaaaatgccgcctctcacacctctttctatatcatatgagaaacttctcccaatgatccaaggcttgtccgacttcaagtggcctagacctattgcaacggacccatctacaagggatcgtagcaggagatgtgccttccacaaagatcatggccatacaacagagacgtgccgatctttccagtatttggtcgaaagactcataaaagctgggcatttgaagcaatacctccgcacggatacggggggtagggacgtttcccagcacaacaaccctggagcccctagggccccagttgccaccaaggccgttatcaactatattaacggaggaccatctgacgaggagtatgactccagacggaaaaggcaaaaattgttgcgggctgcatcaatacgcgaacgcattaattccatccggccgggtctaactggagagggtcctcgccccatagatggaacaataatttttccaccagtagatcccacccggacactacaaccacatcgcgacgccctcatcctgtccctagaaattggggacttcgatgtgcggcgtatcttagttgacccaggcagttcagccgacctggtgcaagcatcagtcgttggccacatgggacatagtctcacgggccttgaaaaccccggacgaatcttatccggattcaacggatcatcaaccacatccttgggagatatcatactgccggtccaagctggcccagtcactctcaatgtacagttttcagtagtacaggagctatcacccttcaatatcattttggggcgcacatggctccactacatgaaagctatcccctctacataccatcaaatggtgatttttcttacccacgatggtcaaatcgacttatatggcagccaattagccgctcgccagtgctatcagatagcacgtgaagcaggggccaaccaggaggatgcatctccccctgaacccagcattgcacgcgaccaatagcaattattgggtccgacggacaaagatcccccggcagcagatcccttacaaacaatccaaatttcggaggaaagcgatcacctcacgaacatcagttccctcatgacacaagaagagactcagggcatgcaaaatatcctcagacagaaccatgacatcttcgcgtgggcacattctgacatgaagggaattcatccctccattacatctcacaggcttaacgtcgtttcaactgccagacccgtccgacagaggattaggcgcttccacccagatagacaaagagtcatccggaacgagattgacaaattgctcgaagccggattcatcagagaagtttcttatccggattggttggcaaacgtagtcgtggtacccaaaaaagaaggaaaatggcgagtttgtgtagattacaccaatctgaacaatgcgtgtccaaaagacagttttcccttgccgcgaatagatcagattgtggattccacttccgggcaagggatgctctctttcttggatgccttctccggatatcatcaaatccccatgtccccggatgacgaagaaaaaatagcattcataacaccacacgacctctattgttataaagtcatgccattcggactcaaaaatgctggcgccacttatcaaagattgatgactaaaatcttcaaacctctgataggccactcagtagaggtttatattgacgatatcgtggttaaaagcaaaactcgggagcagcatatccttcatttgcaagaggttttttacctcttacgaaggtatggcatgaagctaaatccttctaaatgcgcctttggcgtaagtgccagaaaatttctaggatttatggtcagccaaaggggcatagaagtcagcccggatcaagtcaaagcagtcatggaaacaccccctcccaggaacaagaaagagttacagcgcctcacaggcaaactcgttgcattagggcgtttcatagcccgcttcattgatgagttgcgacccttcttcttggcgatacgaaaagctggaacgcacgggtggacggacaattgccaaaacgctttggaaagaattaaacattatcttatgcaaccacccatcttgagcagccccatcccaaaggagaaattatacatgtatctagcagtgtcagaatgggcaatcagtgccgttctattccgctgcccatcacccaaggagcagaagcccatctactatgtcagcagggcattggcagacgtagaaaccagatattcaaaaatggagctaatatccttagcccttcgaagcgctgcccaaaagctccggccctattttcaagcccacccggtgatcgtgttgactgatcaaccccttcgcaacattctgcacaagccagatttaaccgggagaatgctacaatgggccatcgaattaagcgagtttgggattgaattccaaccaaggttgtccatgaaaggccaggtaatggctgacttcgtgctagaatattcccgaaaacccggccaacatgaaggatcaaggaaaaaagaatggtggactctgcgagttgacggagcctcacgttcatcaggctctggagtcgggctcttattgcagtccccaactggggaacatttggaacaagccatccggctgggattctccgcctctaacaatgaagcagaatatgaggctatcttgtccggattggacctcgcccttgctctatccgtctccaaactccggatcttcagcgactcacaactcgtggtaaaacatgtccaggaggaatatgaggctaaggacgcacgcatggcgcgatacttagctaaagtaagaaacaccttgcagcaattcactgaatggacaatcgaaaaaatcaagcgagctgacaataggcgcgctgacgctttggccggcatagctgcctccctctctatcaaggaagccattctactgcccatacatgtgcaaaccaacccctctgtctcagaaatttcaatctgcagtaccactgaggcaccccaagcggacgaccaagaatggatgaatgatatcacagaatatatccggacaggaactctacccggagatcccaaacaggcacataaagtccgggtgcaagctgcccgttttaccttaattggggggcacctgtacaagcgatccttcacaggaccatatcttcggtgcctagggcattcagaagctcaatatgtgttggctgagttacatgagggaatatacggaaatcattcgggaggacgatctctggcacatagagcccattcacaaggatactattggccaacaatgaagaaagaggcggcagcatatgtcaaaagatgtgataaatgtcaaaggtatgctcccatcccacatatgccatcaacaacgttaaaatcaatctcaggtccatggcccttcgcacagtggggcatggacatagtgagacctctcccaactgcacctgcccaaaagaaatttttgcttgttgccacagattatttcagtaagtgggtggaagctgaagcatatgctagcaccaaagataaggatgtcaccaagtttgtatggaaaaatattatctgccgttttggaattccccaaaccatcatagccgacaatggtccacaattcgatagcattgcatttagaaatttctgttcagagctaaacatccggaactcatactccacaccacgatatcctcaaagcaatgggcaagcagaagccacaaacaagaccctaatcactgccttaaagaaaaggcttgagcaagccaaaggaaaatgggtcgaggagctgcccggcgtcctatgggcctaccgaactacacccggacggccaacaggaaatactcctttcgctcttgcatatggaatggacgccgtcattcctatcgaaataggtctccctactatctggaccaacgcagcaaaacaaagtgatgcaaacatgcaattaggaagaaatttggattggacggatgaagtgagggaaagcgcgtccatccggatggcagattatcaacaaagggcatcagcacattacaatcgcaaagtgagaccaagaagcttaaaaaatggtacgctagtccttagaaaattttttgaaaatactactgaagtaggtgcagggaagttccaagccaattgggaaggaccttatatagtgtctaaagcaagtgacaatggagcctatcatttacaaaagctagacggaactccattacttagaccatggaatgtgtctaatttaaagcagtattatcaataagaaagccaaggcaagttcaaatgagaaaagaagtatttttattgacatagctaaaaatatgtacaaaagaggtctccggaccacaaaaatacaaaaggaagattacagaagaaattgcaaaaagagaaactgtcagggagcaggcttgtcataaagtttcttttcttcacccggaggaattgaaggcacgtcccgcttgataccgttcttcttcatgcagcaccgatacccaaagatgaacgtatcatccacctgttttttatattctgcttcaatttcctccctctctacagcaaactcccgttctagctcctctttttgtgcaaccaagcgcagctttaaatcttccctctgtttcttttcaatagaaacttctgtccggagttgcctcacctcccccctcagttgggttatctcatctgctgcctcatgcaggcggtcggccgctgattcctcctgactctttgcctcagccaaatccacccggagggcttcattttcttttcgaatggtggataagccagcttcagcctcttccagtttcaggcgcagttggttttcgctttctttatgccaagaggcaaagatcctcatataatcagtggtccgcagcaaatcaaaaaagagatcgtggtgttgggccatgccgcggagaccactcaccagctgacacacaaacaagcaatgcaaagttacaaatcataccacaacaacacaacaaaatgataaaacaatcaaaaagcacgaaaaacagttttaccatttccacattttcgaacatctgggctgagggcatgatatcaggcgagccgggtggaatctctttcagcttatcttccaactccgcataactaaaagagttgtcggaggagcaagatgcgtcattaacaggtccacccccggatgaagcagcagaaggcgatttttgttccgagtgaggggccccaacatttccggccggatgggcctccccaggtgcaacctcagccgggaccaccatcgggacggatggagcctcagttaccatctccacttcgcccccctccggatgagcgtcgtgggcagatgagcaactgacttcaatctcttgctaccgctcttgaagctgcccgtggagtccggacttcagatcgcgcaccgagcgaggcttctttgaggatggccccgtcactagaacaagagccagcaggttcggatcgtcagagggctgactctggctttctgctcccacttcctccatgggggtcgcgcaaatggcctcagctgcatcggcatccggattgcaggagtccggatggttgacagacgcagcttcctcagccaaattcactagacgcgcaactgctgccaaggaggtgcttgagtggttcagccccgcaaggtgtccggacccgcttgagatggaatgaggggcagcgttcactggctcctctatcattacctccgcctcgtgaataatgggcgaaggagcaaattccatgggagaactgggcttcttcaagtccttcccatttttcttcaccagcttcctctttttcgctggaattttctctggaggagagtctgcgtcacgattctgtccgggagccttccgaagggtcccttcgttcttcttcttctcccgatcctccaggagaagtcgacattttttaacgtcagcctcttttaacgcctcgtaaatcgggaaatccttcacagtataatgctccccaggaacaacttcctttggcatcttcctggggagaatgttgatgacatattctcggggctcccggacgaccgccgtcaggttccgcgcggtcagcagcgtcttgcactgcctctccttggcgtctatctcgaacaatttgctgagacagccaaaggacgccttctccacccagtccacaaggtggcccctcaattccggacctacattaacagcaacaaaagatgagaatgccatccggaagagtcaacaaaaaatcaacaaagtaaaaaccggacaatagtcacaaagcaaacctacccggaaccaccaaggagtagtttggagagaaaggcctcgccggatgctccaaaagccccgcccatgcaccccggaccaccacgtgtcccgtcgcccctccctttgtcgaatctggcagctccgtcaccagttggagggagggcaggtgtgcggacatgctgaagatgtcatttttgaccttcttcaaagaatacacaaaaaacacctccagcagcgtgagatcgaggttgtacagcatgtttatgatgctgcatcccatcagcacccggacgatgttgggatgaataaagacgggtggaatctgggtgaagtggaggaactccttgaacaacgccagcagagggaaccggagccccgcgttgaattgttccttcgagaagatgatagttttttcttctgctttctcagaaggaacaagcaccctcccattcagcaattcgatagccacgtcatttgggatacagaaccgttcccggaattccttcgcgtccaacttatctatcgcttttccacgaacctcggtaccccgagaagatgaaacagtcttttttgcagccatttcttaccacaaacacaagcccaaaactacacacaagaagcgcaatgggtaaaacacaagtaacccagaaaatacacaaaccctaacccgaacaaatcagaaaccaaaaccaacaaaacgcaacaaaaggaacagcaatagcaaaagtacacgtaccaaatgaagctctgaagaagaaaggtcgtcgtttcgaatacaagaacaccagcagcaaacaaatgtcgcgacacaaaatcaccggtacaaaagtgttttcgagtttctttactcagaaaaagcaaaggacgcaacaaaagcaagaagaagaaaggatctcaggaaaatgcagtagaaaaaatgaaaaagaaggtacagtacgatatttatagggggaccagccctcggaaagccaaacgtccaaacagcgctatcattgaaacaacatgctgcctgggaattcccagagtcgccggctcatcataaatgccttttatggcttccgcaccccctctcgccacgtggctcagtcagacgaagagacctcttcaaaattcaaaaagccagttattttttaacccgcccattttttggcaaaataggcaagttaaaaaggggggcaatgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttcagcttcttctgtccaaggaagagcaaacgacgctgacagagcatagacatccggacaaccttcataatgtatctgctccactatacaatccggatagtcagcatgtgaccatccggatttaatcgtccggatcatcaattaaagtaaagcaagtcttacacgctatcacgacaaccagccatggcccacatcccatcatctgcagagtgaaaggacgggtcgaggtgacaacaagtcacttcccacgatcattctacatgatcatttcccacgatatctagacagcagcatcacctaccacggtttctgacagccgccagtagggtggcgatgaccatgctgcctccgaatgtcatcatgacaaacataaaatatctcctcgccattaatgagaggaacagtacccctgaagctgtatatatatgccttcgcacgaagaagaaggggatcctcctggtaacttcttaatacctggtaaaaggccaactaatttatattcctctctctaaccatggctaacaaaaccatcggaggatgcgtccagacaccctgtccggatgccttcttgcaggaatgacgactggatcaaaaacctttatgagttgagatcacgcgtccatccatctggttactacgtggaccgccaaagacgcgaggtaacaacaacacacaccctttgtccatgaactccagcgcatttggaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaaacaagaaatttccacatagttacatcggtaaaaagaaaataagttgaaaatggaaaattatggtgccaattttggatatgatagaatgacttcaatgtttgcctataatggttaattataatattaacacatctctctttctagcttgttagttaatattgacacatctctatttttagggccctatttggtaattggtaattgttttttaaaacaattgtaaaaaatagtttttaaaaactattttatagtgt

SEQ ID NO: 37 - DNA, 2287 bp, synthetic construct
  source 1..2287: mol_type = other DNA
tgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttccaaacgacgctgacagagcatagacatccggacaaccttcataatgtatctgctccactatacaatccggatagtcagcatgtgaccatccggatttaatcgtccggatcatcaattaaagtaaagcaagtcttacacgctatcacgacaaccagccatggcccacatcccatcatctgcagagtgaaaggacgggtcgaggtgacaacaagtcacttcccacgatcattctacatgatcatttcccacgatatctagacagcagcatcacctaccacggtttctgacagccgccagtagggtggcgatgaccatgctgcctccgaatgtcatcatgacaaacataaaatatctcctcgccattaatgagaggaacagtacccctgaagctgtatatatatgccttcgcacgaagaagaaggggatcctcctggtaacttcttaatacctggtaaaaggccaactaatttatattcctctctctaaccatggctaacaaaaccatcggaggatgcgtccagacaccctgtccggatgccttcttgcaggaatgacgactggatcaaaaacctttatgagttgagatcacgcgtccatccatctggttactacgtggaccgccaaagacgcgaggtaacaacaacacacaccctttgtccatgaactccagcgcatttggaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaa

SEQ ID NO: 38 - DNA, 2287 bp, synthetic construct
  source 1..2287: mol_type = other DNA
tgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttcagcttcttctgtccaaggaagagcaaacgacgctgacagagcatagacatccggacaaccttcataatgtatccgctccactatacaatccggatagtcagcatgtgaccatccggatttaatcgtccggatcatcaattaaagtaaagcaagtcttacacgctatcacgacaaccagccatggcccacgtcccatcatctgcagagtgaaaggacgggtcgaggtgacaacaagtcacttcccacgatcattctacatgatcatttccacctaccacggtttctgacagccgccagtagggtggcgatgaccatgctgcctccgaatgtcatcatgacaaacataaaatatctcctcgccattaatgagaggaacagtacccctgaagctgtatatatatgccttcgcacgaagaagaaggggatcctcctggtaacttcttaatacctggtaaaaggccaactaatttatattcctctctctaaccatggctaacaaaaccatcggaggatgcgtccagacaccctgtccggatgccttcttgcaggaatgacgactggatcaaaaacctttatgagttgagatcacgcgtccatccatctggttactacgtggaccgccaaagacgcgaggtaacaacaacacacaccctttgtccatgaactccagcgcatttggaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaa

SEQ ID NO: 39 - DNA, 2287 bp, synthetic construct
  source 1..2287: mol_type = other DNA
tgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttcagcttcttctgtccaaggaagagcaaacgacgctgacagagcatagacatccggacaaccttcataatgtatccgctccactatacaatccggatagtcagcatgtgaccatccggatttaatcgtccggatcatcaattaaagtaaagcaagtcttacacgctatcacgacaaccagccatggcccacgtcccatcatctgcagagtgaaaggacgggtcgaggtgacaacaagtcacttcccacgatcattctacatgatcatttcccacgatatctagacagcagcatcacctaccacggtttctgacagccgccagtagggtggcgatgaccatgctgcctccgaatgtcatcatgacaaacataaaatatctcctcgccattaatgagaggaacagtacccctgaagctgtatatatatgccttcgcacgaagaagaaggggatcctcctggtaacttcttaatacctggtaaaaggccaactgatttatattcctctctctgaccatggctaacaaaaccatcggaggatgcgtccggacaccctgtccggatgccttcttgcaggaatgacgactggatcaaaaacctttacatctggttactacgtggaccgccaaagacgcgaggtaacaacaacacacaccctttgtccatgaactccagcgcatttggaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaa

SEQ ID NO: 40 - DNA, 1666 bp, synthetic construct
  source 1..1666: mol_type = other DNA
tgtagggacccctccctctgggaaacacgtggcacgcacctcacagtgacacgcagcacgtgttatcagccggaccatcatcatccggattcccttaaggatacgcatgatgatggttctcctatccggaccgcctcaaggaaaagcacatgacgtttccaaagacgcgaggtaacaacaacacacaccctttgtccatgaactccagcgcatttggaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaa

SEQ ID NO: 41 - DNA, 2629 bp, synthetic construct
  source 1..2629: mol_type = other DNA
aatacacaccgattgaatagtttaggtttagaaaatgatatatttggaataacaattttttttaattaagaaaaaaaagaaggataaaaatgatattataaaagtttcattaaagcctataatattcccacataaatttaaaaacgtcttctccaagaagatatcttattgctgcctctatattaagctttttttttttttttttttcccttcattaacatcaaaaggtctttggagaaaatgatgcaacgacctgcagaattaagaagttaatgatgtgcaaaatcataactttcttctatttttattaacaataaaatttttttcttaacactactaatccaaatattcacttcaaaactaatatttgttattttattaatttttaaaaatataataatttaatatctacacattacttattttatcaaaattttcaaaggttcatgtgaaaattttatacttttatcaatttccatcatttttcaaattttaaaacaattttaatttatatgcaaatttctacagatatttccataaaactcgaaaatcagtgagggtaacaaagtcaatttttttaacattgggtatatgtatatatttgagggaattgtcagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgcgagccagaggcatatcctaatgattgtacctttcctcactgacaattcccattaattagctgctgccactgcatagcggctataatataatggtagaggacccatggagctttccttttcagtgaacatgggttagtcgacaaaagaaaatgttcaagttgaaagaggagcggtggccctcaaaggttcccatcacttggttgctttttgtctaaggaaacagtggtatcagaatccaaatcttctacgtaatgtcccattcatcctaccaatgtccatatgaattcctctggacgttaaaaaatggttgcacgtggttgccttcaggatcacaccagtttatacatttggaccacaaaatagagattgttcatcaaggatactagtcagcaattaattcctaaatatctcttatgacaaagccagtaatgcaccataagaaacgtgtcgaataaaccaattaggggtctggtgtccgagtcatgagatagaacaggttcgaggttgttatatatcaatcaataattagagaaggagccggtctcttgtgttgagttgactcgatggagagcttaggagttagaaagggtgcatggatccaagaagaggatgttctcctgaggaaatgcattgagaaatatggagaaggaaagtggcatctggttcccctccgagcaggtaacatgaaagagaaagggatcagtatttatttgtgtttttttacttctgttttgcttaaagagtttcattttcttgagtttgcagggttgaatagatgccgaaaaagctgcaggttgagatggctcaattatttgaagccggatatcaagagaggagagtttgcattagacgaggttgatctcatgattaggcttcacaatttgttggggaacaggcaagtctataataactcaagtactagcttgataatgatattatattagttctgaagctgttcagaacttacaaaagagctgttcagttgatactttgtctgatgttgtgcgtgtatagatggtccttgattgcgggtaggcttccagggaggactgctaatgatgtcaagaactattggcatagtcaccacttcaaaaaggaggttcagttccaggaagaagggagagataaaccccaaacacattctaaaaccaaagctataaagcctcaccctcacaagttctccaaagccttgccaaggtttgaactaaaaactacagctgtggatacttttgacacacaagtcagtacttccaggaagccatcatccacttcaccacaaccgaatgatgacatcatatggtgggaaagcctgttagctgagcatgctcaaatggatcaagaaactgacttttcggcttctggagagatgcttatcgcaagcctcaggacagaagaaactgcaacacagaaaaagggacccatggatggtatgattgaacaaatccagggaggtgagggtgattttccatttgatgtgggcttctgggatacacccaacacacaagtaaatcacttgatctgatttagaccatttcaatcttaatgtttaagttcaaaagttgttcttgcattcatgctctactccctaatcattttatgttccaaagttgttaatcttagaaataaaaacaaaaataataaatatatcaatatgtataatggtggatagggatgtgagcagtggatgcaaactcaagcattgaatgaatggttaagattaatgaacccaatcatttgaagtgacaaaagaaagacattttaaaagattcttaacaatatgaaaaagttgtttcatatttctatcggtttccaaccattaattagacggatggaccattgaaatatctcatgcatattgaattaataaa